(19) United States
(12) Patent Application Publication
(10) Pub. No.: US 2004/0210962 A1
(43) Pub. Date: Oct. 21, 2004

(54) Implementation of a mitochondrial mutator

(75) Inventors: Sally Mackenzie, Lincoln, NE (US); Ricardo Vilela Abdelnoor, Lincoln, NE (US)

Correspondence Address: JONDLE & ASSOCIATES P.C., 9085 EAST MINERAL CIRCLE, SUITE 200, CENTENNIAL, CO, 80112, US

(73) Assignee: The Board of Regents of the University of Nebraska

(21) Appl. No.: 10/806,038

(22) Filed: Mar. 22, 2004

Related U.S. Application Data
(60) Provisional application No. 60/456,318, filed on Mar. 20, 2003.

(51) Int. Cl. A01H 1/00; C12N 15/82; C12Q 1/68; C07H 21/04; C12N 5/04

(52) U.S. Cl. 800/278; 536/23.6; 435/6; 435/69.1; 435/419; 435/468; 530/370

(57) ABSTRACT

Plant MSH1 polynucleotides and polypeptides are described. Also described are methods for the use and modulation of such MSH1 polynucleotides and polypeptides.

### CROSS-REFERENCE TO RELATED APPLICATIONS

This application claims priority under 35 U.S.C. §119 from U.S. Application Ser. No. 60/456,318, filed Mar. 20, 2003, which is incorporated herein in its entirety by reference.

### GOVERNMENT LICENSE RIGHTS

The U.S. Government has a paid-up license in this invention and the right in limited circumstances to require the patent owner to license others on reasonable terms as provided for by the terms of the contracts awarded by the National Science Foundation and the Department of Energy.

### TECHNICAL FIELD

This invention relates to using molecular and evolutionary techniques to identify polynucleotide and polypeptide sequences corresponding to commercially relevant traits in domesticated plants.

### BACKGROUND OF THE INVENTION

The plant mitochondrial genome is retained in a multipartite structure that arises by a process of repeat-mediated homologous recombination. Low frequency ectopic recombination also occurs, often producing sequence chimeras, aberrant open reading frames, and novel subgenomic DNA molecules. This genomic plasticity may distinguish the plant mitochondrion from mammalian and fungal types. In plants, relative copy number of recombination-derived subgenomic DNA molecules within mitochondria is controlled by nuclear genes, and a genomic shifting process can result in their differential copy number suppression to near-undetectable levels. We have cloned a nuclear gene that regulates mitochondrial substoichoimetric shifting in Arabidopsis. The CHM gene was shown to encode a protein related to the MutS protein of E. coli that is involved in mismatch repair and DNA recombination. We postulate that the process of substoichiometric shifting in plants may be a consequence of ectopic recombination suppression or replication stalling at ectopic recombination sites to effect molecule-specific copy number modulation.

Argument for the mitochondrion as a central regulator of cellular functions has become increasingly persuasive in the past several years, as information expands detailing cell metabolic functions (Golden & Melov, (2001) Mech. Aging Dev. 122, 1577-1589; Naviaux (2000) Eur. J.Ped. 159, 5219-5226), programmed cell death (Ravagnan, et al. (2002)1 Cell. Physiol. 192,131-137), and intracellular signaling (Epstein et al. (2001) Molec. Biol.Cell. 12,297-308). The disclosures of Golden & Melov, Naviaux, and all other patents and publications referred to herein, are incorporated herein in their entirety by reference. In higher plants, mitochondrial functions and behavior have clearly been influenced by the plant cell's unique context. Co-evolution of mitochondria and chloroplasts has permitted economy of function via protein dual-targeting (Small, et al. (1998) Plant Molec. Biol. 38, 265-277, Peeters & Small (2001) Biochim. Biophys. Acta 1541, 54-63), genome capacity and coding have been altered (Knoop & Brennicke (2002) Crit. Rev. Plant Sci. 21,111-126), and the mitochondrial genomes of plants have acquired structural and maintenance features distinct from their animal counterparts.

The plant mitochondrial genome appears to be organized as a collection of small circular and large, circularly-permuted linear molecules (Oldenburg & Bendich (2001) Molec. Biol. 310, 549-562; Backert, et al. (1997) Trend Plant Sci. 2, 477-483), not unlike what has been postulated for yeast (Maleszka, et al. (1991) EMBO J. 10, 3923-3929; Lecrenier & Foury (2000) Gene 246,37-48). DNA replication may be conducted by a rolling circle mechanism, and experimental difficulties identifying replication origins have led to the suggestion of recombination-mediated replication initiation (Backert & Borner (2000) Curr. Genet. 37, 304-314). In fact, a distinct feature of plant mitochondrial genome organization is the prominent role of recombination.

High frequency inter- and intra-molecular recombination is detected within the higher plant mitochondrial genome at large repeated sequences that can be readily identified by physical mapping (Fauron, et al. (1995) Trends Genet. 11, 228-235). Their presence in direct orientation permits the subdivision of the genome into a collection of molecules, each containing only a portion of the genetic information. More intriguing, however, is the common observation in plants of intragenic ectopic recombination events that can occur at sites containing as few as seven nucleotides of homology (Andre, et al. (1992) Trends Genet. 8, 128-132). Ectopic recombination results in expressed gene chimeras that cause cytoplasmic male sterility, plant variegation and other aberrant phenotypes (Mackenzie & Mcintosh (1999) Plant Cell 11, 571-585; Sakamoto, et al. (1996) Plant Cell 8, 1377-1390).

A phenomenon rendering the plant mitochondrial genome unusually variable in structure is termed substoichiometric shifting. First reported in maize (Small, et al. (1987) EMBO J. 6, 865-869) as the stable presence of subgenomic mitochondrial DNA molecules within the genome at near-undetectable levels, the process appears to be highly dynamic. Mitochondrial genomic shifting involves rapid and dramatic changes in relative copy number of portions of the mitochondrial genome over one generation's time (Janska, et al. (1998) Plant Cell 10,1163-1180). These substoichiometric forms have been estimated at levels as low as one copy per every 100-200 cells (Arrieta-Montiel, et al. (2001) Genetics 158, 851-864). Generally the rapid shifting process involves only a single subgenomic DNA molecule, often containing recombination-derived chimeric sequences, and the process is apparently reversible (Janska, et al., ibid., Kanazawa, et al. (1994) Genetics 138, 865-870). Genomic shifting can alter plant phenotype because the process activates or silences mitochondrial sequences located on the shifted molecule. Observed phenotypic changes have included plant tissue culture properties (Kanazawa, et al., ibid.), leaf variegation and distortion (Sakamoto, et al., ibid.), and spontaneous reversion to fertility in cytoplasmic male sterile crop plants (Janska, et al., ibid., Smith, et al. (1991) Theor. Appl. Genet. 81,793-798). It has been postulated that substoichiometric shifting may have evolved to permit the species to create and retain mitochondrial genetic variation in a silenced but retrievable form (Small, et al. (1989) Cell 58, 69-76).

Mitochondrial substoichiometric shifting has been shown in at least two cases to be under nuclear gene control, involving the Fr gene in Phaseolus vulgaris (Mackenzie & Chase (1990) Plant Cell 2, 905-912) and the CHM gene in Arabidopsis (Martinez-Zapater, et al. (1992) Plant Cell 4, 889-899; Redei (1973) Mut. Res. 18,149-162). Mutation of the nuclear CHM gene results in a green-white leaf variegation that, in subsequent generations, displays maternal inheritance (Redei, ibid.). The appearance of the variegation phenotype is accompanied by a specific rearrangement (Martinez-Zapater, et al., ibid.) that includes amplification of a mitochondrial DNA molecule encoding a chimeric sequence (Sakamoto, et al., ibid.). Genetic analysis suggests that the wildtype form of CHM actively suppresses copy number of the subgenomic molecule carrying the chimeric sequence. Loss of proper function of the CHM gene, characterized by two available EMS-derived mutant alleles chm1-1, chm1-2 (Redei, ibid.) and a tissue culture-derived mutant allele chm1-3 (Martinez-Zapater, et al., ibid.), results in rapid and specific copy number amplification of the subgenomic molecule, producing the consequent leaf variegation. It is not clear whether the copy number amplification or suppression of a single subgenomic molecule occurs by differential replication or a recombination mechanism.

### SUMMARY OF THE INVENTION

The present invention provides an isolated nucleic acid molecule selected from the group consisting of: a nucleic acid molecule comprising a nucleic acid sequence selected from the group consisting of SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and SEQ ID NO:45; a nucleic acid molecule comprising at least a portion of any of these nucleic acid molecules; a complement of a any of these nucleic acid molecules; and a nucleic acid molecule comprising an allelic variant of a nucleic acid molecule comprising any of these nucleic acid sequences.

In some embodiments, the nucleic acid molecule is a plant nucleic acid molecule, a nucleic acid molecule selected from the group consisting of Arabadopsis, Oryza, Glycine, Hordeum, Zea, Medicago, Allium, Citrus, Solanum, Sorghum, Saccharum, Nicotiana, Lycopersicon, Triticum, Zinnia, and Phaseolus nucleic acid molecules, a nucleic acid molecule selected from the group consisting of: a nucleic acid molecule comprising a nucleic acid sequence that encodes a protein having an amino acid sequence selected from the group consisting of SEQ ID NO:3, SEQ ID NO:7, SEQ ID NO.:9, SEQ ID NO.:12, SEQ ID NO.:15, SEQ ID NO:17, SEQ ID NO.:19, SEQ ID NO.:22,SEQ ID NO.:24, SEQ ID NO.:26, SEQ ID NO.:31,SEQ ID NO:33, SEQ ID NO.:35, SEQ ID NO.:40, SEQ ID NO.:42, SEQ ID NO:44, SEQ ID NO:47, and SEQ ID NO:65; and a nucleic acid molecule comprising an allelic variant of a nucleic acid molecule encoding a protein having any of said amino acid sequences.

The present invention also provides an isolated MSH1 protein. In some embodiment, the protein is encoded by a plant MSH1 nucleic acid molecule that hybridizes to the complement of a nucleic acid molecule having a nucleic acid sequence SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, or SEQ ID NO:45 under stringent hybridization conditions. In some embodiments, the protein is SEQ ID NO:3, SEQ ID NO:7, SEQ ID NO.:9, SEQ ID NO.:12, SEQ ID NO.:15, SEQ ID NO:17, SEQ ID NO.:19, SEQ ID NO.:22,SEQ ID NO.:24, SEQ ID NO.:26, SEQ ID NO.:31,SEQ ID NO:33, SEQ ID NO.:35, SEQ ID NO.:40, SEQ ID NO.:42, SEQ ID NO:44, SEQ ID NO:47 or SEQ ID NO:65, or a protein comprising at least a portion of an amino acid sequence selected from the group consisting of SEQ ID NO:3, SEQ ID NO:7, SEQ ID NO.:9, SEQ ID NO.:12, SEQ ID NO.:15, SEQ ID NO:17, SEQ ID NO.:19, SEQ ID NO.:22,SEQ ID NO.:24, SEQ ID NO.:26, SEQ ID NO.:31,SEQ ID NO:33, SEQ ID NO.:35, SEQ ID NO.:40, SEQ ID NO.:42, SEQ ID NO:44, SEQ ID NO:47 and SEQ ID NO:65.

The present invention also provides a method to identify a compound capable of inhibiting MSH1 activity of a plant, said method comprising: contacting an isolated plant MSH1 nucleic acid molecule selected from the group consisting of SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and SEQ ID NO:45 with a putative inhibitory compound which, in the absence of said compound, said plant MSH1 nucleic acid molecule has the activity of suppressing ectopic recombination; and determining if said putative inhibitory compound inhibits said activity. In some embodiments, the putative inhibitory compound is a RNA molecule suspected of having RNAi activity. The invention also provides compounds identified by the method

Further provided is a method for identification of plant mutants arising from mitochondrial ectopic recombination comprising providing a plant, suppressing expression of an MSH1-homologous gene in the plant, and detecting an aberrant phenotype,

whereby a plant mutant is identified. In some embodiments, the suppression is effected by a compound identified by the above-described method. In some embodiments, the aberrant phenotype is cytoplasmic male sterility. The invention also provides plant mutants identified by the method of claim 12.

### BRIEF DESCRIPTION OF THE FIGURES

FIG. 1. Positional cloning of the CHM candidate locus. The use of molecular markers permitted the establishment of a genetic map (A) and identification of the intervening overlapping bacterial artificial chromosome clones for physical mapping (B) All physical mapping information was derived from the Arabidopsis Genome Initiative (50). High resolution mapping with three markers permitted delimitation of the locus to a 80-kb interval contained within a single bacterial artificial chromosome clone (C) A gene candidate was identified within the interval based on predicted mitochondrial targeting features. The candidate CHM locus contains 22 exons (D) with two MutS-like conserved intervals denoted by red lines. Analysis of two EMS-derived mutants, chm1-1 and chm1-2, and one tissue culture-derived mutant chm1-3, as well as two TDNA insertion mutations (T1 and T2), provided definitive evidence of CHM identity (E). The numbers in parentheses in (A) correspond to the number of recombinants identified between the marker and the gene.

FIG. 2. Alignment of AtMSH1 with MutS and MutS homologs. The amino acid sequence alignment was performed using the ClustalW software and includes the MutS sequence from E. coli, MSH1 from Saccharomyces cerevisiae, and AtMSH6 and CHM (AtMSH1) from Arabidopsis. (A) Alignment of the region of the DNA-binding domain that encompasses the conserved motif for mismatch recognition and DNA binding. (B) Alignment of a portion of the ATPase domain. The characteristic motifs for this domain are indicated by red lines. M1—Walker motif; M2—ST motif; M3—DE motif (Walker B motif); M4—TH motif (Obmolova, et al. (2000) Nature 407, 703-710; Lamers, et al. (2000) Nature, 407, 711-717). The asterisks (*) indicate residues that are identical and the arrow indicates the site of amino acid substitution in mutant chur1-3.

FIG. 3. Alignment of MSH proteings.

### DETAILED DESCRIPTION OF THE INVENTION

The present invention provides a plant nuclear gene and corresponding gene product, in Arabidopsis thaliana that influences mitochondrial genome organization. The gene is designated AtMSH1, and it is believed to suppress ectopic (illegitimate) recombination of the mitochondrial genome. The present invention provides for isolated MSH1 proteins, isolated MSH1 nucleic acid molecules, antibodies directed against MSH1 proteins and other inhibitors of MSH1 activity. As used herein, the terms isolated MSH1 proteins and isolated MSH1 nucleic acid molecules refers to MSH1 proteins and esterase nucleic acid molecules derived from plants and, as such, can be obtained from their natural source or can be produced using, for example, recombinant nucleic acid technology or chemical synthesis. The term “plant” refers to an individual living plant or population of same, a species, subspecies, variety, cultivar or strain. In some preferred embodiments, the domesticated organism is a plant selected from the group consisting of maize, wheat, rice, sorghum, tomato or potato, or any other domesticated plant of commercial interest. A “plant” is any plant at any stage of development, including a seed plant. Also included in the present invention is the use of these proteins, nucleic acid molecules, antibodies and inhibitors to generate transgenic plants, and mutant plants, as well as in other applications, such as those disclosed below.

The present invention is the result of studies investigating the unusual plant phenomenon of mitochondrial subtoichiometric shifting and the role of the nuclear gene CHM. This gene, located on chromosome III, was shown to encode a protein that is targeted to mitochondria and that has homology to a yeast mitochondrial MutS protein. A summary of this investigation is provided in the EXAMPLES section.

MSH1 proteins and nucleic acid molecules of the present invention have utility because they represent novel targets for modulation which would effect mitochondrial ectopic recombination. The products and processes of the present invention are advantageous because they enable the express and inhibition of processes that involve MSH1. While not being bound by theory, it is believed these newly discovered proteins have contributed adaptive advantage by a strategy that may be unique to the Plant Kingdom.

A. MSH1 Polypeptides

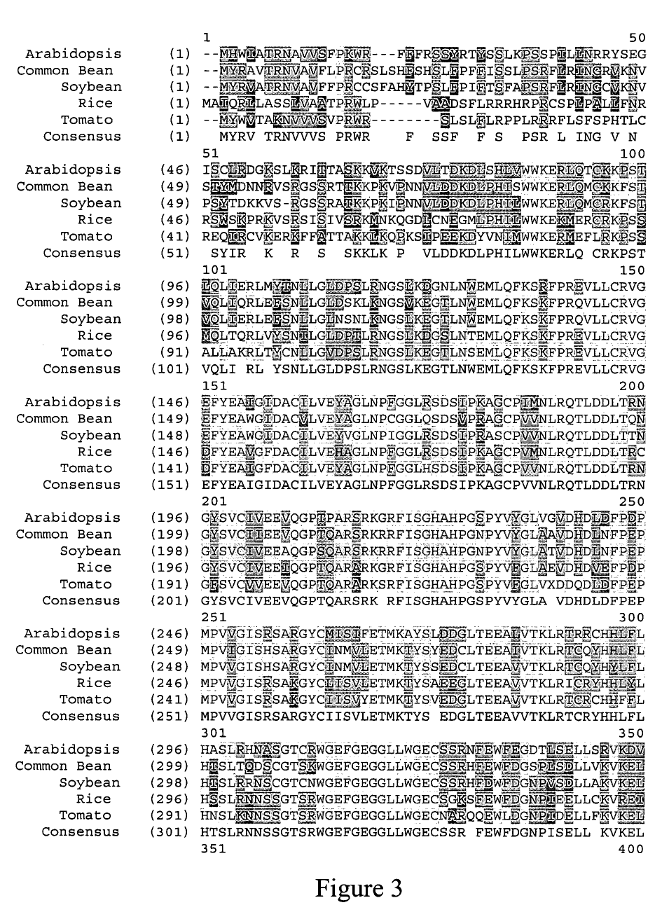

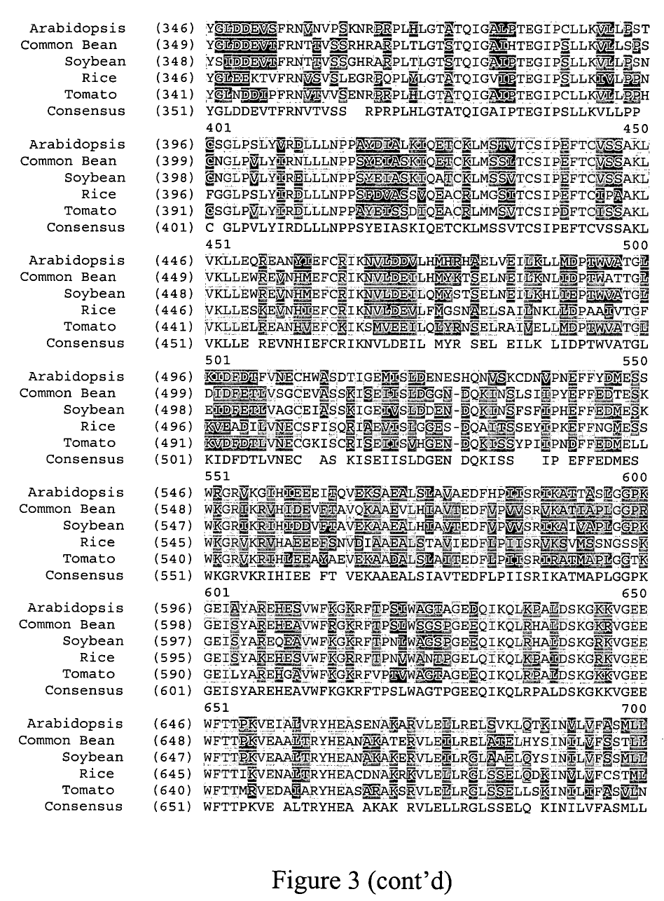

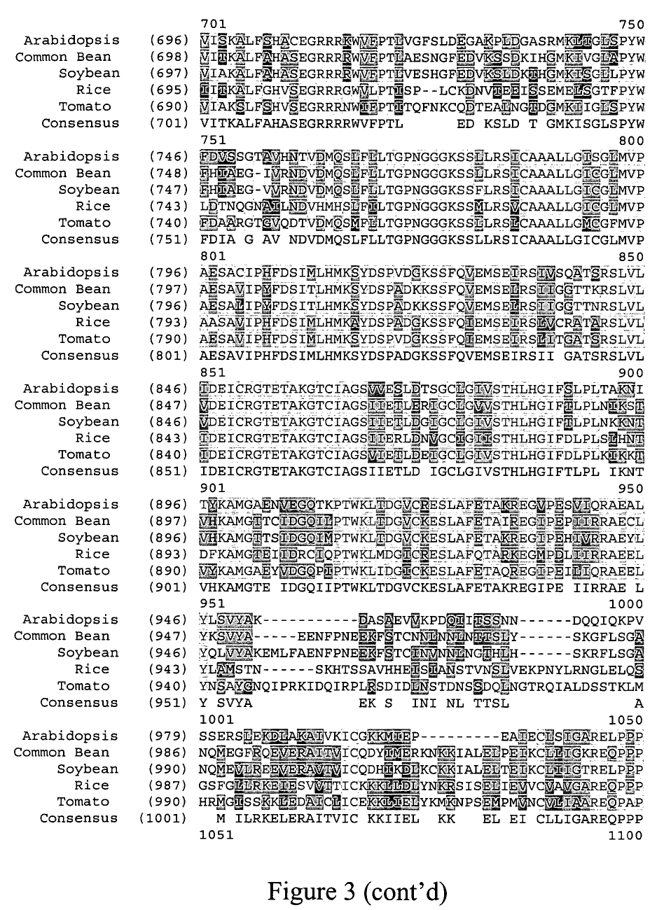

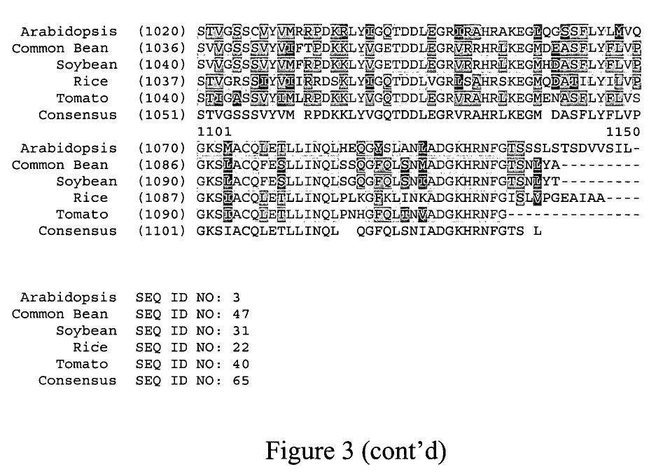

One embodiment of the present invention is an isolated plant MSH1 polypeptide. As used herein, an MSH1 polypeptide, in one embodiment, is a polypeptide that is related to (i.e., bears structural similarity to) the A. thaliana polypeptide of about 1118 amino acids and having the sequence depicted in FIG. 3 (SEQ ID NO: 3). The original identification of such a polypeptide is detailed in the Examples.

A preferred MSH1 polypeptide is encoded by a polynucleotide that hybridizes under stringent hybridization conditions to a gene encoding an MSH1 polypeptide (i.e., an A. thaliana gene). It is to be noted that the term “a” or “an” entity refers to one or more of that entity; for example, a gene refers to one or more genes or at least one gene. As such, the terms “a” (or “an”), “one or more” and “at least one” can be used interchangeably herein. It is also to be noted that the terms “comprising,” “including,” and “having” can be used interchangeably.

As used herein, stringent hybridization conditions refer to standard hybridization conditions under which polynucleotides, including oligonucleotides, are used to identify molecules having similar nucleic acid sequences. Such standard conditions are disclosed, for example, in Sambrook et al., MOLECULAR CLONING: A LABORATORY MANUAL, Cold Spring Harbor Labs Press, 1989. Examples of such conditions are provided in the Examples section of the present application.

As used herein, an A. thaliana AtMSH1 gene includes all nucleic acid sequences related to a natural A. thaliana AtMSH1 gene such as regulatory regions that control production of the A. thaliana AtMSH1 polypeptide encoded by that gene (such as, but not limited to, transcription, translation or post-translation control regions) as well as the coding region itself. In one embodiment, an A. thaliana AtMSH1 gene includes the nucleic acid sequence SEQ ID NO:1. Nucleic acid sequence SEQ ID NO:X represents the deduced sequence of a cDNA (complementary DNA) polynucleotide, the production of which is disclosed in the Examples. It should be noted that since nucleic acid sequencing technology is not entirely error-free, SEQ ID NO:1 (as well as other sequences presented herein), at best, represents an apparent nucleic acid sequence of the polynucleotide encoding an A. thaliana AtMSH1 polypeptide of the present invention.

In another embodiment, an A. thaliana AtMSH1 gene can be an allelic variant that includes a similar but not identical sequence to SEQ ID NO:1. During higher plant evolution, natural allelic variation for the MSH1 locus likely revealed the adaptive advantage that arises from sporadic copy number modulation of mitochondrial genomic variants. Some of these variants, when amplified, condition male sterility that could facilitate advantageous outcrossing activity in natural populations (Arrieta-Montiel, et al., ibid.). An allelic variant of an A. thaliana AtMSH1 gene including SEQ ID NO: 1 is a locus (or loci) in the genome whose activity is concerned with the same biochemical or developmental processes, and/or a gene that that occurs at essentially the same locus as the gene including SEQ ID NO:1, but which, due to natural variations caused by, for example, mutation or recombination, has a similar but not identical sequence. Because genomes can undergo rearrangement, the physical arrangement of alleles is not always the same. Allelic variants typically encode polypeptides having similar activity to that of the polypeptide encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given cultivar or strain since the genome is diploid and/or among a population comprising two or more cultivars or strains.

According to the present invention, an isolated, or biologically pure, polypeptide, is a polypeptide that has been removed from its natural milieu. As such, “isolated” and “biologically pure” do not necessarily reflect the extent to which the polypeptide has been purified. An isolated MSH1 polypeptide of the present invention can be obtained from its natural source, can be produced using recombinant DNA technology or can be produced by chemical synthesis. An MSH1 polypeptide of the present invention may be identified by its ability to perform the function of natural MSH1 in a functional assay. By “natural MSH1 polypeptide,” it is meant the full length MSH1 polypeptide of A. thaliana. The phrase “capable of performing the function of a natural MSH1 in a functional assay” means that the polypeptide has at least about 10% of the activity of the natural polypeptide in the functional assay. In other embodiments, the MSH1 polypeptide has at least about 20% of the activity of the natural polypeptide in the functional assay. In other embodiments, the MSH1 polypeptide has at least about 30% of the activity of the natural polypeptide in the functional assay. In other embodiments, the MSH1 polypeptide has at least about 40% of the activity of the natural polypeptide in the functional assay. In other embodiments, the MSH1 polypeptide has at least about 50% of the activity of the natural polypeptide in the functional assay. In other embodiments, the polypeptide has at least about 60% of the activity of the natural polypeptide in the functional assay. In other embodiments, the polypeptide has at least about 70% of the activity of the natural polypeptide in the functional assay. In other embodiments, the polypeptide has at least about 80% of the activity of the natural polypeptide in the functional assay. In still other embodiments, the polypeptide has at least about 90% of the activity of the natural polypeptide in the functional assay. Examples of functional assays are detailed elsewhere in this specification.

As used herein, an isolated plant MSH1 polypeptide can be a full-length polypeptide or any homologue of such a polypeptide. Examples of MSH1 homologues include MSH1 polypeptides in which amino acids have been deleted (e.g., a truncated version of the polypeptide, such as a peptide), inserted, inverted, substituted and/or derivatized (e.g., by glycosylation, phosphorylation, acetylation, myristylation, prenylation, palmitoylation, amidation and/or addition of glycerophosphatidyl inositol) such that the homolog has natural MSH1 activity.

In one embodiment, when the homologue is administered to an animal as an immunogen, using techniques known to those skilled in the art, the animal will produce a humoral and/or cellular immune response against at least one epitope of a natural MSH1 polypeptide. MSH1 homologues can also be selected by their ability to perform the function of MSH1 in a functional assay.

Plant MSH1 polypeptide homologues can be the result of natural allelic variation or natural mutation. MSH1 polypeptide homologues of the present invention can also be produced using techniques known in the art including, but not limited to, direct modifications to the polypeptide or modifications to the gene encoding the polypeptide using, for example, classic or recombinant DNA techniques to effect random or targeted mutagenesis.

In accordance with the present invention, a mimetope refers to any compound that is able to mimic the ability of an isolated plant MSH1 polypeptide of the present invention to perform the function of an MSH1 polypeptide of the present invention in a functional assay. Examples of mimetopes include, but are not limited to, anti-idiotypic antibodies or fragments thereof, that include at least one binding site that mimics one or more epitopes of an isolated polypeptide of the present invention; non-polypeptideaceous immunogenic portions of an isolated polypeptide (e.g., carbohydrate structures); and synthetic or natural organic molecules, including nucleic acids, that have a structure similar to at least one epitope of an isolated polypeptide of the present invention. Such mimetopes can be designed using computer-generated structures of polypeptides of the present invention. Mimetopes can also be obtained by generating random samples of molecules, such as oligonucleotides, peptides or other organic molecules, and screening such samples by affinity chromatography techniques using the corresponding binding partner.

The minimal size of an MSH1 polypeptide homologue of the present invention is a size sufficient to be encoded by a polynucleotide capable of forming a stable hybrid with the complementary sequence of a polynucleotide encoding the corresponding natural polypeptide. As such, the size of the polynucleotide encoding such a polypeptide homologue is dependent on nucleic acid composition and percent homology between the polynucleotide and complementary sequence as well as upon hybridization conditions per se (e.g., temperature, salt concentration, and formamide concentration). It should also be noted that the extent of homology required to form a stable hybrid can vary depending on whether the homologous sequences are interspersed throughout the polynucleotides or are clustered (i.e., localized) in distinct regions on the polynucleotides. The minimal size of such polynucleotides is typically at least about 12 to about 15 nucleotides in length if the polynucleotides are GC-rich and at least about 15 to about 17 bases in length if they are AT-rich. Preferably, the polynucleotide is at least 12 bases in length.

As such, the minimal size of a polynucleotide used to encode an MSH1 polypeptide homologue of the present invention is from about 12 to about 18 nucleotides in length. There is no limit, other than a practical limit, on the maximal size of such a polynucleotide in that the polynucleotide can include a portion of a gene, an entire gene, or multiple genes, or portions thereof. Similarly, the minimal size of an MSH1 polypeptide homologue of the present invention is from about 4 to about 6 amino acids in length, with preferred sizes depending on whether a full-length, fusion, multivalent, or functional portions of such polypeptides are desired. Preferably, the polypeptide is at least 30 bases in length.

Any plant MSH1 polypeptide is a suitable polypeptide of the present invention. Suitable plants from which to isolate MSH1 polypeptides (including isolation of the natural polypeptide or production of the polypeptide by recombinant or synthetic techniques) include maize, wheat, barley, rye, millet, chickpea, lentil, flax, olive, fig almond, pistachio, walnut, beet, parsnip, citrus fruits, including, but not limited to, orange, lemon, lime, grapefruit, tangerine, minneola, and tangelo, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, and woody plants such as coniferous and deciduous trees, with soybean, tomato, potato, rice, wheat, and barley being preferred.

A preferred plant MSH1 polypeptide of the present invention is a compound that when expressed or modulated in a plant, is capable of suppressing ectopic recombination of the mitochondrial genome.

One embodiment of the present invention is a fusion polypeptide that includes an MSH1 polypeptide-containing domain attached to a fusion segment. Inclusion of a fusion segment as part of a MSH1 polypeptide of the present invention can enhance the polypeptide's stability during production, storage and/or use. Depending on the segment's characteristics, a fusion segment can also act as an immunopotentiator to enhance the immune response mounted by an animal immunized with an MSH1 polypeptide containing such a fusion segment. Furthermore, a fusion segment can function as a tool to simplify purification of an MSH1 polypeptide, such as to enable purification of the resultant fusion polypeptide using affinity chromatography. A suitable fusion segment can be a domain of any size that has the desired function (e.g., imparts increased stability, imparts increased immunogenicity to a polypeptide, and/or simplifies purification of a polypeptide). It is within the scope of the present invention to use one or more fusion segments. Fusion segments can be joined to amino and/or carboxyl termini of the MSH1-containing domain of the polypeptide. Linkages between fusion segments and MSH1-containing domains of fusion polypeptides can be susceptible to cleavage in order to enable straightforward recovery of the MSH1-containing domains of such polypeptides. Fusion polypeptides are preferably produced by culturing a recombinant cell transformed with a fusion polynucleotide that encodes a polypeptide including the fusion segment attached to either the carboxyl and/or amino terminal end of a MSH1-containing domain.

Exemplary fusion segments for use in the present invention include a glutathione binding domain; a metal binding domain, such as a poly-histidine segment capable of binding to a divalent metal ion; an immunoglobulin binding domain, such as Polypeptide A, Polypeptide G, T cell, B cell, Fc receptor or complement polypeptide antibody-binding domains; a sugar binding domain such as a maltose binding domain from a maltose binding polypeptide; and/or a “tag” domain (e.g., at least a portion of β-galactosidase, a strep tag peptide, other domains that can be purified using compounds that bind to the domain, such as monoclonal antibodies). Other fusion segments suitable for use in the invention include metal binding domains, such as a poly-histidine segment; a maltose binding domain; a strep tag peptide.

Preferred plant MSH1 polypeptides of the present invention are Arabadopsis MSH1 polypeptides, soybean MSH1 polypeptides, tomato MSH1 polypeptides, rice MSH1 polypeptides, and common bean MSH1 polypeptides. Other preferred plant MSH polypeptides include corn MSH1 polypeptides, wheat MSH1 polypeptides, sugar cane MSH1 polypeptides, medicago MSH1 polypeptides, onion MSH1 polypeptides, orange MSH1 polypeptides, zinnia MSH1 polypeptides, tobacco MSH1 polypeptides, and barleyMSH1 polypeptides.

One preferred A. thaliana AtMSH1 polypeptide of the present invention is a polypeptide encoded by an A. thaliana polynucleotide that hybridizes under stringent hybridization conditions with complements of polynucleotides represented by SEQ ID NO:1. Such an AtMSH1 polypeptide is encoded by a polynucleotide that hybridizes under stringent hybridization conditions with a polynucleotide having nucleic acid sequence SEQ ID NO:1.

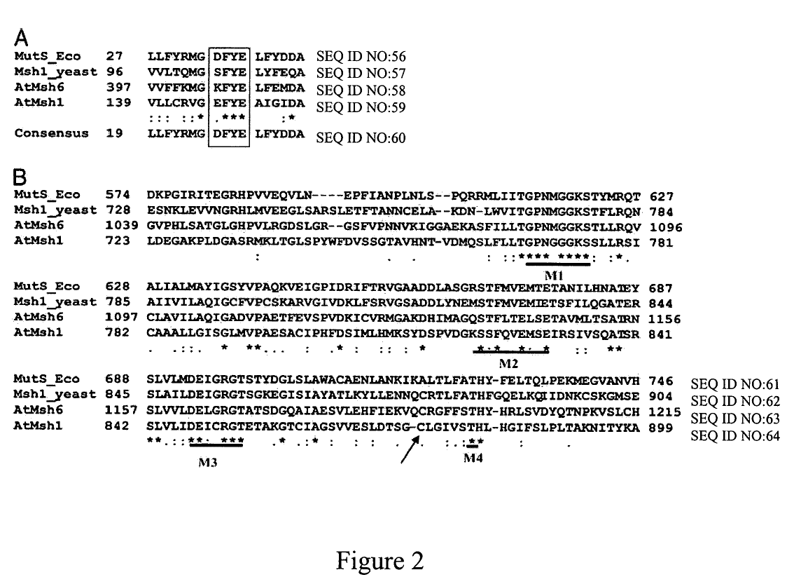

Inspection of AtMSH1 genomic nucleic acid sequences indicates that the genes comprise several regions, including an ATP-binding domain, comprised of four well conserved motifs designated M1-M4 (Obmolova, et al., ibid.; FIG. 2B), and a DNA binding domain (aa 129-206) containing the aromatic doublet (FY) motif.

Translation of SEQ ID NO:1 suggests that the A. thaliana AtMSH1 polynucleotide includes an open reading frame. The reading frame encodes an A. thaliana AtMSH1 polypeptide of about 1118 amino acids, the deduced amino acid sequence of which is represented herein as SEQ ID NO:3, assuming an open reading frame having an initiation (start) codon spanning from about nucleotide 124 through about nucleotide 126 of SEQ ID NO:1 and a termination (stop) codon spanning from about nucleotide 3478 through about nucleotide 3480 of SEQ ID NO:1.

Similarly, translation of SEQ ID NO:20 suggests that the Oryza sativa MSH1 polynucleotide includes an open reading frame. The reading frame encodes an Oryza sativa MSH polypeptide of about 1132 amino acids, the deduced amino acid sequence of which is represented herein as SEQ ID NO:22, assuming an open reading frame having an initiation (start) codon spanning from about nucleotide 1 through about nucleotide 3 of SEQ ID NO:22 and a termination (stop) codon spanning from about nucleotide 3394 through about nucleotide 3396 of SEQ ID NO:20.

Similarly, translation of SEQ ID NO:29 suggests that the Glycine max MSH1 polynucleotide includes an open reading frame. The reading frame encodes an Glycine max MSH polypeptide of about 1130 amino acids, the deduced amino acid sequence of which is represented herein as SEQ ID NO:31, assuming an open reading frame having an initiation (start) codon spanning from about nucleotide 1 through about nucleotide 3 of SEQ ID NO:29 and a termination (stop) codon spanning from about nucleotide 3391 through about nucleotide 3393 of SEQ ID NO:20.

Similarly, translation of SEQ ID NO:38 suggests that the Lycopersicon esculentum MSH1 polynucleotide includes an open reading frame. The reading frame encodes an Lycopersicon esculentum MSH polypeptide of about 1124 amino acids, the deduced amino acid sequence of which is represented herein as SEQ ID NO:40, assuming an open reading frame having an initiation (start) codon spanning from about nucleotide 1 through about nucleotide 3 of SEQ ID NO:38 and a termination (stop) codon spanning from about nucleotide 3369 through about nucleotide 3371 of SEQ ID NO:20.

Similarly, translation of SEQ ID NO:45 suggests that the Phaseolus vulgaris MSH1 polynucleotide includes an open reading frame. The reading frame encodes an Phaseolus vulgaris MSH polypeptide of about 1126 amino acids, the deduced amino acid sequence of which is represented herein as SEQ ID NO:47, assuming an open reading frame having an initiation (start) codon spanning from about nucleotide 1 through about nucleotide 3 of SEQ ID NO:45 and a termination (stop) codon spanning from about nucleotide 3379 through about nucleotide 3381 of SEQ ID NO:20.

Additional EST sequences having at least 60% sequence identity to a portion of SEQ ID NO.:1 or a complement of SEQ ID NO:1 have been found. These include MSH1 polynucleotides from corn (SEQ ID NO:l1), potato (SEQ ID NO:18), wheat (SEQ ID NO:41), sugar cane (SEQ ID NO:32 and SEQ ID NO:34), medicago (SEQ ID NO:13), onion (SEQ ID NO:14), orange (SEQ ID NO:16), zinnia (SEQ ID NO:43), tobacco (SEQ ID NO:36), and barley (SEQ ID NO:6, SEQ ID NO:8, SEQ ID NO:10). Polypeptides encoded by the foregoing nucleic acid molecules can be deduced using methods well known in the art. In general, the polynucleotide or its complement is aligned with the Arabidopsis AtMSH1 polynucleotide, a reading frame is determined, and the resulting polypeptide sequence is translasted. Polypeptides encoded by the foregoing nucleic acid molecules or their complements include corn (SEQ ID NO:12), potato (SEQ ID NO:19), wheat (SEQ ID NO:42), sugar cane (SEQ ID NO:33 and SEQ ID NO:35), onion (SEQ ID NO:15), orange (SEQ ID NO:17), zinnia (SEQ ID NO:44), and barley (SEQ ID NO:7, SEQ ID NO:9), and consensus (SEQ ID NO:65).

Comparison of the various A. thaliana, soybean, corn, tomato, potato, rice, wheat, common bean, sugar cane, medicago, onion, orange, zinnia, tobacco, and barley MSH1 nucleic acid sequences and amino acid sequences described herein indicates that these species of plants possess similar MSH1 genes and polypeptides. The nucleotide sequences of the coding region of MSH1 from the various plants have >60% sequence identity when compared to each other, which makes clear that they are homologous.

Finding this degree of identity between soybean, corn, tomato, potato, rice, wheat, common bean, sugar cane, medicago, onion, orange, zinnia, tobacco, and barley MSH1 nucleic acid sequences and amino acid sequences supports the ability to obtain any plant MSH1 polypeptide and polynucleotide given the polypeptide and nucleic acid sequences disclosed herein.

These plant MSH1 polypeptides, and the polynucleotides that encode them, represent novel compounds with utility in ectopic recombination of the mitochondrial genome.

Preferred plant MSH1 polypeptides of the present invention include polypeptides comprising amino acid sequences that are at least about 30%, preferably at least about 50%, more preferably at least about 75% and even more preferably at least about 90% identical to one or more of the amino acid sequences disclosed herein for A. thaliana AtMSH1 polypeptides of the present invention. More preferred plant MSH1 polypeptides of the present invention include: polypeptides encoded by at least a portion of SEQ ID NO.:1, SEQ ID NO.:20, SEQ ID NO.:29, SEQ ID NO.:38 and/or SEQ ID NO:45 and, as such, have amino acid sequences that include at least a portion of SEQ ID NO:3, SEQ ID NO.:22, SEQ ID NO.:31, SEQ ID NO.:40 and/or SEQ ID NO:47; polypeptides encoded by at least a portion of SEQ ID NO:1, SEQ ID NO.:20, SEQ ID NO.:29, SEQ ID NO.:38 and/or SEQ ID NO:45 and, as such, have amino acid sequences that include at least a portion of SEQ ID NO:3, SEQ ID NO.:22, SEQ ID NO.:31, SEQ ID NO.:40 and/or SEQ ID NO:47. Also preferred are polypeptides that have amino acid sequences that include at least a portion of SEQ ID NO:7, SEQ ID NO.:9, SEQ ID NO.:12, SEQ ID NO.:15, SEQ ID NO:17, SEQ ID NO.:19, SEQ ID NO.:24, SEQ ID NO.:26, SEQ ID NO:33, SEQ ID NO.:35, SEQ ID NO.:42, and/or SEQ ID NO:44; and polypeptides encoded by at least a portion of SEQ ID NO:6, SEQ ID NO.:8, SEQ ID NO.:10, SEQ ID NO.:11, SEQ ID NO:13, SEQ ID NO.:14, SEQ ID NO.:16, SEQ ID NO.:18, SEQ ID NO:23, SEQ ID NO.:25, SEQ ID NO.:26, SEQ ID NO.:27, SEQ ID NO.:28, SEQ ID NO.:30, SEQ ID NO:32, SEQ ID NO.:34, SEQ ID NO.:36, SEQ ID NO.:37, SEQ ID NO.:41, and/or SEQ ID NO:43, or a complement of any of the foregoing SEQ ID NO:s. As used herein, “at least a portion” of a polynucleotide or polypeptide means a portion having the minimal size characteristics of such sequences, as described above, or any larger fragment of the full length molecule, up to and including the full length molecule. For example, a portion of a polynucleotide may be 12 nucleotides, 13 nucleotides, 14 nucleotides, 15 nucleotides, and so on, going up to the full length polynucleotide. Similarly, a portion of a polypeptide may be 4 amino acids, 5 amino acids, 6 amino acids, 7 amino acids, and so on, going up to the full length polypeptide. The length of the portion to be used will depend on the particular application. As discussed above, a portion of a polynucleotide useful as hybridization probe may be as short as 12 nucleotides. A portion of a polypeptide useful as an epitope may be as short as 4 amino acids. A portion of a polypeptide that performs the function of the full-length polypeptide would generally be longer than 4 amino acids.

Particularly preferred plant MSH1 polypeptides of the present invention are polypeptides that include SEQ ID NO:3, SEQ ID NO:7, SEQ ID NO.:9, SEQ ID NO.:12, SEQ ID NO.:15, SEQ ID NO:17, SEQ ID NO.:19, SEQ ID NO.:22,SEQ ID NO.:24, SEQ ID NO.:26, SEQ ID NO.:31,SEQ ID NO:33, SEQ ID NO.:35, SEQ ID NO.:40, SEQ ID NO.:42, SEQ ID NO:44, SEQ ID NO:47 and/or SEQ ID NO:65 (including, but not limited to the encoded polypeptides, full-length polypeptides, processed polypeptides, fusion polypeptides and multivalent polypeptides thereof) as well as polypeptides that are truncated homologues of polypeptides that include at least portions of the aforementioned SEQ ID NOs. Examples of methods to produce such polypeptides are disclosed herein, including in the Examples section.

Plant MSH1 polypeptides may have DNA binding and ATPase activities. Identification of the chm1-3 mutation as a cysteine-tyrosine substitution within the predicted ATP binding domain does suggest the importance of this region to protein function. Substitution of the bulkier tyrosine would likely create distortion in the region, affecting ATP binding or hydrolysis.

Mismatch repair components appear to be involved in not only the binding and excision of nucleotide mismatches during the replication process, but also suppression of ectopic recombination (Harfe & Jinks-Robertson (2000) Annu. Rev. Genet. 34, 359-399; Chen & Jinks-Robertson (1999) Genetics 151,1299-1313). Investigation of the mitochondrial substoichometric shifting phenomenon suggests two alternative models for the influence of MSH1. It is conceivable that the MSH1 gene has shared or relinquished its mismatch repair function, such that its primary role in the plant mitochondrial genome is to regulate non-homologous recombination. Disruption of MSH1 could, thus, result in the enhancement of intra-molecular ectopic recombination activity detected as apparent amplification of novel mitochondrial DNA forms. A possible weakness in this model arises in reports that several plant systems with mitochondrial DNA molecules susceptible to shifting appear to be derived from a DNA exchange that involved at least one molecular form no longer present in high copy number. Some also appeared to contain unique sequences. Therefore, the shifted molecules were thought to replicate autonomously (Andre, et al., ibid; Kanazawa, et al., ibid; , Janska & Mackenzie (1993) Genetics 135, 869-879).

If mitochondrial DNA molecules that undergo shifting are, in fact, replicated autonomously, an alternative model for molecule-specific substoichiometric shifting might apply. The Arabidopsis MSH1 product likely participates as a component of the DNA replication apparatus. Mitochondrial DNA molecules subject to copy number shifting may have originated by earlier ectopic recombination events during the evolution of the lineage. In this case, the resulting chimeric sites might serve to trigger a process of site-specific replication stalling by the MSH1 protein during vegetative growth.

Both models assume that the replicative form of the mitochondrial genome within meristematic (undifferentiated) tissues differs from that of vegetative (somatic). Hence, stoichiometric shifting events in vegetative tissues do not condition irreversible loss of the suppressed genetic information. Presumably, the complete mitochondrial genetic complement is retained within the transmitting (meristematic) tissues (Arrieta-Montiel, et al., Janska & Mackenzie, ibid.).

B. MSH1 Polynucleotides

One embodiment of the present invention is an isolated plant polynucleotide that hybridizes under stringent hybridization conditions with an A. thaliana AtMSH1 gene. The identifying characteristics of such genes are heretofore described. A polynucleotide of the present invention can include an isolated natural plant MSH1 gene or a homologue thereof, the latter of which is described in more detail below. A polynucleotide of the present invention can include one or more regulatory regions, full-length or partial coding regions, or combinations thereof. The minimal size of a polynucleotide of the present invention is the minimal size that can form a stable hybrid with one of the aforementioned genes under stringent hybridization conditions. Suitable and preferred plants are disclosed above.

In accordance with the present invention, an isolated polynucleotide is a polynucleotide that has been removed from its natural milieu (i.e., that has been subject to human manipulation). As such, “isolated” does not reflect the extent to which the polynucleotide has been purified. An isolated polynucleotide can include DNA, RNA, or derivatives of either DNA or RNA.

An isolated plant MSH1 polynucleotide of the present invention can be obtained from its natural source either as an entire (i.e., complete) gene or a portion thereof capable of forming a stable hybrid with that gene. An isolated plant MSH1 polynucleotide can also be produced using recombinant DNA technology (e.g., polymerase chain reaction (PCR) amplification, cloning) or chemical synthesis. Isolated plant MSH1 polynucleotides include natural polynucleotides and homologues thereof, including, but not limited to, natural allelic variants and modified polynucleotides in which nucleotides have been inserted, deleted, substituted, and/or inverted in such a manner that such modifications do not substantially interfere with the polynucleotide's ability to encode an MSH1 polypeptide of the present invention or to form stable hybrids under stringent conditions with natural gene isolates.

A plant MSH1 polynucleotide homologue can be produced using a number of methods known to those skilled in the art (see, for example, Sambrook et al., ibid.). For example, polynucleotides can be modified using a variety of techniques including, but not limited to, classic mutagenesis techniques and recombinant DNA techniques, such as site-directed mutagenesis, chemical treatment of a polynucleotide to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of polynucleotides and combinations thereof. Polynucleotide homologues can be selected from a mixture of modified nucleic acids by screening for the function of the polypeptide encoded by the nucleic acid (e.g., ability to elicit an immune response against at least one epitope of an MSH1 polypeptide, ability to suppress ectopic recombination in a transgenic plant containing an MSH1 gene and/or by hybridization with an A. thaliana AtMSH1 gene.

An isolated polynucleotide of the present invention can include a nucleic acid sequence that encodes at least one plant MSH1 polypeptide of the present invention, examples of such polypeptides being disclosed herein. Although the phrase “polynucleotide” primarily refers to the physical polynucleotide and the phrase “nucleic acid sequence” primarily refers to the sequence of nucleotides on the polynucleotide, the two phrases can be used interchangeably, especially with respect to a polynucleotide, or a nucleic acid sequence, being capable of encoding an MSH1 polypeptide. As heretofore disclosed, plant MSH1 polypeptides of the present invention include, but are not limited to, polypeptides having full-length plant MSH1 coding regions, polypeptides having partial plant MSH1 coding regions, fusion polypeptides, multivalent protective polypeptides and combinations thereof.

At least certain polynucleotides of the present invention encode polypeptides that selectively bind to immune serum derived from an animal that has been immunized with an MSH1 polypeptide from which the polynucleotide was isolated.

A preferred polynucleotide of the present invention, when suppressed in a suitable plant, is capable of generating economically useful mutant plants. As will be disclosed in more detail below, such a polynucleotide can be, or encode, an antisense RNA, a molecule capable of triple helix formation, a ribozyme, or other nucleic acid-based compound.

One embodiment of the present invention is a plant MSH1 polynucleotide that hybridizes under stringent hybridization conditions to an MSH1 polynucleotide of the present invention, or to a homologue of such an MSH1 polynucleotide, or to the complement of such a polynucleotide. A polynucleotide complement of any nucleic acid sequence of the present invention refers to the nucleic acid sequence of the polynucleotide that is complementary to (i.e., can form a complete double helix with) the strand for which the sequence is cited. It is to be noted that a double-stranded nucleic acid molecule of the present invention for which a nucleic acid sequence has been determined for one strand, that is represented by a SEQ ID NO, also comprises a complementary strand having a sequence that is a complement of that SEQ ID NO. As such, polynucleotides of the present invention, which can be either double-stranded or single-stranded, include those polynucleotides that form stable hybrids under stringent hybridization conditions with either a given SEQ ID NO denoted herein and/or with the complement of that SEQ ID NO, which may or may not be denoted herein. Methods to deduce a complementary sequences are known to those skilled in the art. Preferred is an MSH1 polynucleotide that includes a nucleic acid sequence having at least about 60 percent, at least about 65 percent, preferably at least about 70 percent, more preferably at least about 75 percent, more preferably at least about 80 percent, more preferably at least about 85 percent, more preferably at least about 90 percent and even more preferably at least about 95 percent homology with the corresponding region(s) of the nucleic acid sequence encoding at least a portion of an MSH1 polypeptide. Particularly preferred is an MSH1 polynucleotide capable of encoding at least a portion of an MSH1 polypeptide that naturally is present in plants.

Particularly preferred MSH1 polynucleotides of the present invention hybridize under stringent hybridization conditions with at least one of the following polynucleotides: SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and/or SEQ ID NO:45, or to a homologue or complement of such polynucleotide.

A preferred polynucleotide of the present invention includes at least a portion of nucleic acid sequence SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and/or SEQ ID NO:45 that is capable of hybridizing (i.e., that hybridizes under stringent hybridization conditions) to an A. thaliana AtMSH1 gene of the present invention, as well as a polynucleotide that is an allelic variant of any of those polynucleotides. Such preferred polynucleotides can include nucleotides in addition to those included in the SEQ ID NOs, such as, but not limited to, a full-length gene, a full-length coding region, a polynucleotide encoding a fusion polypeptide, and/or a polynucleotide encoding a multivalent protective compound.

The present invention also includes polynucleotides encoding a polypeptide including at least a portion of SEQ ID NO:3, polynucleotides encoding a polypeptide having at least a portion of SEQ ID NO:7, polynucleotides encoding a polypeptide having at least a portion of SEQ ID NO:9, polynucleotides encoding a polypeptide having at least a portion of SEQ ID NO:12, polynucleotides encoding a polypeptide having at least a portion of SEQ ID NO:15, polynucleotides encoding a polypeptide having at least a portion of SEQ ID NO:17, polynucleotides encoding a polypeptide having at least a portion of SEQ ID NO:19, polynucleotides encoding a polypeptide having at least a portion of SEQ ID NO:22, polynucleotides encoding a polypeptide having at least a portion of SEQ ID NO:24, polynucleotides encoding a polypeptide having at least a portion of SEQ ID NO:26, polynucleotides encoding a polypeptide having at least a portion of SEQ ID NO:31, polynucleotides encoding a polypeptide having at least a portion of SEQ ID NO:33, polynucleotides encoding a polypeptide having at least a portion of SEQ ID NO:35, polynucleotides encoding a polypeptide having at least a portion of SEQ ID NO:40, polynucleotides encoding a polypeptide having at least a portion of SEQ ID NO:42, polynucleotides encoding a polypeptide having at least a portion of SEQ ID NO:42, polynucleotides encoding a polypeptide having at least a portion of SEQ ID NO:44, polynucleotides encoding a polypeptide having at least a portion of SEQ ID NO:47, and/or polynucleotides encoding a polypeptide having at least a portion of SEQ ID NO:65, including polynucleotides that have been modified to accommodate codon usage properties of the cells in which such polynucleotides are to be expressed.

Knowing the nucleic acid sequences of certain plant MSH1 polynucleotides of the present invention allows one skilled in the art to, for example, (a) make copies of those polynucleotides, (b) obtain polynucleotides including at least a portion of such polynucleotides (e.g., polynucleotides including full-length genes, full-length coding regions, regulatory control sequences, truncated coding regions), and (c) obtain MSH1 polynucleotides for other plants. Such polynucleotides can be obtained in a variety of ways including screening appropriate expression libraries with antibodies of the present invention; traditional cloning techniques using oligonucleotide probes of the present invention to screen appropriate libraries or DNA; and PCR amplification of appropriate libraries or DNA using oligonucleotide primers of the present invention. Preferred libraries to screen or from which to amplify polynucleotides include libraries such as genomic DNA libraries, BAC libraries, YAC libraries, cDNA libraries prepared from isolated plant tissues, including, but not limited to, stems, reproductive structures/tissues, leaves, roots, and tillers; and libraries constructed from pooled cDNAs from any or all of the tissues listed above. In the case of rice, BAC libraries, available from Clemson University, are preferred. Similarly, preferred DNA sources to screen or from which to amplify polynucleotides include plant genomic DNA. Techniques to clone and amplify genes are disclosed, for example, in Sambrook et al., ibid. and in Galun & Breiman, TRANSGENIC PLANTS, Imperial College Press, 1997.

The present invention also includes polynucleotides that are oligonucleotides capable of hybridizing, under stringent hybridization conditions, with complementary regions of other, preferably longer, polynucleotides of the present invention such as those comprising plant MSH1 genes or other plant MSH1 polynucleotides. Oligonucleotides of the present invention can be RNA, DNA, or derivatives of either. The minimal size of such oligonucleotides is the size required to form a stable hybrid between a given oligonucleotide and the complementary sequence on another polynucleotide of the present invention. Minimal size characteristics are disclosed herein. The size of the oligonucleotide must also be sufficient for the use of the oligonucleotide in accordance with the present invention. Oligonucleotides of the present invention can be used in a variety of applications including, but not limited to, as probes to identify additional polynucleotides, as primers to amplify or extend polynucleotides, as targets for expression analysis, as candidates for targeted mutagenesis and/or recovery, or in agricultural applications to alter MSH1 polypeptide production or activity. Such agricultural applications include the use of such oligonucleotides in, for example, antisense-, triplex formation-, ribozyme- and/or RNA drug-based technologies. The present invention, therefore, includes such oligonucleotides and methods in a plant by use of one or more of such technologies.

The predicted features of the candidate CHM-encoded protein denoted MSH1 suggest that the gene encodes the mitochondrial MSH1 counterpart in higher plants. MSH1 encodes a mitochondrial mismatch repair protein in yeast, though its counterpart in animals has not yet been identified. The CHM candidate sequence showed strongest homology with the Arabidopsis nuclear MSH6 sequence (FIG. 2), consistent with suggestions that nuclear mismatch repair components likely derived from a progenitor to MSH1 (Culligan, et al. (2000) Nucl. Acids Res. 28, 463-471).

Although the predicted CHM candidate protein displayed several features suggesting its involvement in mismatch repair, lines containing mutations in the locus showed no evidence of mitochondrial point mutation accumulation. The primary effect within the mitochondrion appeared to be the reproducible substoichiometric shifting phenomenon. This assumption is based on the observation of identical mitochondrial DNA restriction fragments arising upon substoichiometric shifting in all chm mutants when tested repeatedly (Sakamoto, et al., ibid., Martinez-Zapater, et al., ibid., this report). Moreover, no evidence of progressive decline in plant growth features has been observed over time. The chm1-1 and chm1-2 mutants, reported in the 1970's (Redei, ibid.), appear identical to one another in phenotype and mitochondrial DNA configuration. Although detailed sequence analysis would be required to estimate the incidence of mismatch accumulation in the chm mutants, one would anticipate a random pattern of mitochondrial DNA polymorphism and progressive phenotypic decline in chm mutants were the mismatch accumulation rate enhanced.

Mutation of the MSH1 locus in yeast results in rapid accumulation of mitochondrial genomic rearrangements leading to disruption of mitochondrial function. Interestingly, a reproducible pattern of DNA restriction fragment polymorphism was reported in some of the petit mutants arising in yeast MSH1 mutant strains (Reenan & Kolodner). This observation may be indication that mshl-associated mitochondrial genomic rearrangements are similar in plants and fungi. Alignment between the yeast MSH1 protein and the Arabidopsis CHM (MSH1) candidate shows only 17% amino acid identity overall, with ca. 28% identity within the predicted functional domains for ATP and DNA binding, but with well conserved motifs (FIG. 2). The yeast MSH1 protein has been shown to have both DNA mismatch binding and ATPase activity (Chi & Kolodner (1994) J Biol. Chem. 269,29984-29992; Chi & Kolodner. (1994) J. Biol. Chem. 269, 29993-29997).

C. Recombinant Molecules

The present invention also includes a recombinant vector, which includes at least one plant MSH1 polynucleotide of the present invention, inserted into any vector capable of delivering the polynucleotide into a host cell. Such a vector contains heterologous nucleic acid sequences, that is nucleic acid sequences that are not naturally found adjacent to polynucleotides of the present invention and that preferably are derived from a species other than the species from which the polynucleotide(s) are derived. As used herein, a derived polynucleotide is one that is identical or similar in sequence to a polynucleotide or portion of a polynucleotide, but can contain modifications, such as modified bases, backbone modifications, nucleotide changes, and the like. The vector can be either RNA or DNA, either prokaryotic or eukaryotic, and typically is a virus or a plasmid. Recombinant vectors can be used in the cloning, sequencing, and/or otherwise manipulating of plant MSH1 polynucleotides of the present invention. One type of recombinant vector, referred to herein as a recombinant molecule and described in more detail below, can be used in the expression of polynucleotides of the present invention. Preferred recombinant vectors are capable of replicating in the transformed cell.

Suitable and preferred polynucleotides to include in recombinant vectors of the present invention are as disclosed herein for suitable and preferred plant MSH1 polynucleotides per se. Particularly preferred polynucleotides to include in recombinant vectors, and particularly in recombinant molecules, of the present invention include SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and/or SEQ ID NO:45.

Isolated plant MSH1 polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell that is capable of expressing the polypeptide, the recombinant cell being produced by transforming a host cell with one or more polynucleotides of the present invention. Transformation of a polynucleotide into a cell can be accomplished by any method by which a polynucleotide can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotides of the present invention can remain extrachromosomal or can integrate into one or more sites within a chromosome of the transformed (i.e., recombinant) cell in such a manner that their ability to be expressed is retained. Suitable and preferred polynucleotides with which to transform a cell are as disclosed herein for suitable and preferred plant MSH1 polynucleotides per se. Particularly preferred polynucleotides to include in recombinant cells of the present invention include SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and/or SEQ ID NO:45.

Suitable host cells to transform include any cell that can be transformed with a polynucleotide of the present invention. Host cells can be either untransformed cells or cells that are already transformed with at least one polynucleotide. Host cells of the present invention either can be endogenously (i.e., naturally) capable of producing plant MSH1 polypeptides of the present invention or can be capable of producing such polypeptides after being transformed with at least one polynucleotide of the present invention. Host cells of the present invention can be any cell capable of producing at least one polypeptide of the present invention, and include bacterial, fungal (including yeast and rice blast, Magnaporthe grisea), parasite (including nematodes, especially of the genera Xiphinema, Helicotylenchus, and Tylenchlohynchus), insect, other animal and plant cells.

Suitable host viruses to transform include any virus that can be transformed with a polynucleotide of the present invention, including, but not limited to, rice stripe virus, and echinochloa hoja blanca virus.

In a preferred embodiment, non-pathogenic symbiotic bacteria, which are able to live and replicate within plant tissues, so-called endophytes, or non-pathogenic symbiotic bacteria, which are capable of colonizing the phyllosphere or the rhizosphere, so-called epiphytes, are used. Such bacteria include bacteria of the genera Agrobacterium, Alcaligenes, Azospirillum, Azotobacter, Bacillus, Clavibacter, Enterobacter, Erwinia, Flavobacter, Klebsiella, Pseudomonas, Rhizobium, Serratia, Streptomyces and Xanthomonas. Symbiotic fungi, such as Trichoderma and Gliocladium are also possible hosts for expression of the inventive nucleotide sequences for the same purpose.

A recombinant cell is preferably produced by transforming a host cell with one or more recombinant molecules, each comprising one or more polynucleotides of the present invention operatively linked to an expression vector containing one or more transcription control sequences. The phrase “operatively linked” refers to insertion of a polynucleotide into an expression vector in a manner such that the molecule is able to be expressed in the correct reading frame when transformed into a host cell. As used herein, an expression vector is a DNA or RNA vector that is capable of transforming a host cell and of effecting expression of a specified polynucleotide. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, parasite, insect, other animal, and plant cells. Preferred expression vectors of the present invention can direct gene expression in bacterial, yeast, fungal, insect and mammalian cells and more preferably in the cell types heretofore disclosed.

Recombinant molecules of the present invention may also (a) contain secretory signals (i.e., signal segment nucleic acid sequences) to enable an expressed MSH1 polypeptide of the present invention to be secreted from the cell that produces the polypeptide and/or (b) contain fusion sequences which lead to the expression of polynucleotides of the present invention as fusion polypeptides. Examples of suitable signal segments and fusion segments encoded by fusion segment nucleic acids are disclosed herein. Eukaryotic recombinant molecules may include intervening and/or untranslated sequences surrounding and/or within the nucleic acid sequences of polynucleotides of the present invention. Suitable signal segments include natural signal segments or any heterologous signal segment capable of directing the secretion of a polypeptide of the present invention. Preferred signal and fusion sequences employed to enhance organ and organelle specific expression include, but are not limited to, arcelin-5, see Goossens, A. et. al. The arcelin-5 Gene of Phaseolus vulgaris directs high seed-specific expression in transgenic Phaseolus acutifolius and Arabidopsis plants. Plant Physiology (1999) 120:1095-1104, phaseolin, see Sengupta-Gopalan, C. et. al. Developmentally regulated expression of the bean beta-phaseolin gene in tobacco seeds. PNAS (1985) 82:3320-3324, hydroxyproline-rich glycoprotein , serpin, see Yan, X. et. al. Gene fusions of signal sequences with a modified beta-glucuronidase gene results in retention of the beta-glucuronidase protein in the secretory pathway/plasma membrane. Plant Physiology (1997) 115:915-924, N-acetyl glucosaminyl transferase 1, see Essi, D. et. al. The N-terminal 77 amino acids from tobacco N-acetylglucosaminyltransferase I are sufficient to retain reporter protein in the Golgi apparatus of Nicotiana benthamiana cells. Febs Letters (1999) 453(1-2):169-73, albumin, see Vandekerckhove, J. et. al. Enkephalins produced in transgenic plants using modified 2S seed storage proteins. BioTechnology 7:929-932 (1989) and PR1, see Pen, J. et. al. Efficient production of active industrial enzymes in plants. Industrial Crops and Prod. (1993) 1:241-250, and other sequences as described in the Examples.

Polynucleotides of the present invention can be operatively linked to expression vectors containing regulatory sequences such as transcription control sequences, translation control sequences, origins of replication, and other regulatory sequences that are compatible with the recombinant cell and that control the expression of polynucleotides of the present invention. In particular, recombinant molecules of the present invention include transcription control sequences. Transcription control sequences are sequences which control the initiation, elongation, and termination of transcription. Included are those transcription control sequences which are sufficient to render promoter-dependent gene expression controllable for cell-type specific, tissue-specific or inducible by external signals or agents; such elements may be located in the 5′ or 3′ regions of the native gene. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, fungal, insect and mammalian cells, such as, but not limited to, tac, lac, trp, trc, oxy-pro, omp/lpp, rrnB, bacteriophage lambda (λ) (such as λp_{L} and λp_{R} and fusions that include such promoters), bacteriophage T7, T7lac, bacteriophage T3, bacteriophage SP6, bacteriophage SP01, metallothionein, α-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, poxvirus, adenovirus, cytomegalovirus (such as intermediate early promoters, simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

Particularly preferred transcription control sequences are plant transcription control sequences. The choice of transcription control sequence will vary depending on the temporal and spatial requirements for expression, and also depending on the target species. Thus, expression of the nucleotide sequences of this invention in any plant organ (leaves, roots, seedlings, immature or mature reproductive structures, etc.) or at any stage of plant development is preferred. Although many transcription control sequences from dicotyledons have been shown to be operational in monocotyledons and vice versa, ideally dicotyledonous transcription control sequences are selected for expression in dicotyledons, and monocotyledonous promoters for expression in monocotyledons. However, there is no restriction to the provenance of selected transcription control sequences; it is sufficient that they are operational in driving the expression of the nucleotide sequences in the desired cell.

Preferred transcription control sequences that are expressed constitutively include but are not limited to promoters from genes encoding actin or ubiquitin and the CaMV 35S and 19S promoters. The nucleotide sequences of this invention can also be expressed under the regulation of promoters that are chemically regulated. This enables the MSH1 polypeptide to be synthesized only when the crop plants are treated with the inducing chemicals.

A preferred category of promoters is that which is induced by the physiological state of the plant (i.e. wound inducible, water-stress inducible, salt-stress inducible, disease inducible, and the like). Numerous promoters have been described which are expressed at wound sites and also at the sites of phytopathogen infection. Ideally, such a promoter should only be active locally at the sites of infection, and in this way the MSH1 polypeptides only accumulate in cells in which the accumulation is desired. Preferred promoters of this kind include those described by Stanford et al. Mol. Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Preferred tissue-specific expression patterns include but are not limited to green tissue specific, root specific, stem specific, and flower specific. Promoters suitable for expression in green tissue include many which regulate genes involved in photosynthesis and many of these have been cloned from both monocotyledons and dicotyledons. A preferred promoter is the maize PEPC promoter from the phosphoenol carboxylase gene (Hudspeth & Grula, Plant Molec. Biol. 12: 579-589 (1989)). A preferred promoter for root specific expression is that described by de Framond (FEBS 290: 103-106 (1991); EP 0 452 269 to Ciba-Geigy). A preferred stem specific promoter is that described in U.S. Pat. No. 5,625,136 (to Ciba-Geigy) and which drives expression of the maize trpA gene.

A recombinant molecule of the present invention is a molecule that can include at least one of any polynucleotide heretofore described operatively linked to at least one of any transcription control sequence capable of effectively regulating expression of the polynucleotide(s) in the cell to be transformed, examples of which are disclosed herein.

A recombinant cell of the present invention includes any cell transformed with at least one of any polynucleotide of the present invention. Suitable and preferred polynucleotides as well as suitable and preferred recombinant molecules with which to transfer cells are disclosed herein.

Recombinant cells of the present invention can also be co-transformed with one or more recombinant molecules including plant MSH1 polynucleotides encoding one or more polypeptides of the present invention and one or more other polypeptides useful when expressed in plants.

It may be appreciated by one skilled in the art that use of recombinant DNA technologies can improve expression of transformed polynucleotides by manipulating, for example, the number of copies of the polynucleotides within a host cell, the efficiency with which those polynucleotides are transcribed, the efficiency With which the resultant transcripts are translated, and the efficiency of post-translational modifications. Recombinant techniques useful for increasing the expression of polynucleotides of the present invention include, but are not limited to, operatively linking polynucleotides to high-copy number plasmids, integration of the polynucleotides into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotides of the present invention to correspond to the codon usage of the host cell, deletion of sequences that destabilize transcripts, and use of control signals that temporally separate recombinant cell growth from recombinant enzyme production during fermentation. The activity of an expressed recombinant polypeptide of the present invention may be improved by fragmenting, modifying, or derivatizing polynucleotides encoding such a polypeptide.

Recombinant cells of the present invention can be used to produce one or more polypeptides of the present invention by culturing such cells under conditions effective to produce such a polypeptide, and recovering the polypeptide. Effective conditions to produce a polypeptide include, but are not limited to, appropriate media, bioreactor, temperature, pH and oxygen conditions that permit polypeptide production. An appropriate, or effective, medium refers to any medium in which a cell of the present invention, when cultured, is capable of producing an MSH1 polypeptide of the present invention. Such a medium is typically an aqueous medium comprising assimilable carbon, nitrogen and phosphate sources, as well as appropriate salts, minerals, metals and other nutrients, such as vitamins. The medium may comprise complex nutrients or may be a defined minimal medium. Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant polypeptides of the present invention may either remain within the recombinant cell; be secreted into the fermentation medium; be secreted into a space between two cellular membranes, such as the periplasmic space in E. coli; or be retained on the outer surface of a cell or viral membrane.

The phrase “recovering the polypeptide” refers simply to collecting the whole fermentation medium containing the polypeptide and need not imply additional steps of separation or purification. Polypeptides of the present invention can be purified using a variety of standard polypeptide purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Polypeptides of the present invention are preferably retrieved in “substantially pure” form. As used herein, “substantially pure” refers to a purity that allows for the effective use of the polypeptide as a diagnostic or test compound, and means, with increasing preference, at least 50%, 60%, 70%, 80%, 90%, 95%, or 98% homogeneous.

D. Transfected Plant Cells and Transgenic Plants

With regard to MSH1, particularly preferred recombinant cells are plant cells. By “plant cell” is meant any self-propagating cell bounded by a semi-permeable membrane and containing a plastid. Such a cell also requires a cell wall if further propagation is desired. Plant cell, as used herein includes, without limitation, algae, cyanobacteria, seeds, suspension cultures, embryos, meristematic regions, callus tissue, leaves, roots, shoots, gametophytes, sporophytes, pollen, and microspores.

The particular arrangement of the MSH1 sequence in the transformation vector will be selected according to the type of expression of the sequence that is desired. In some embodiments, expressing MSH1 polypeptides is desirable, while in others, a reduction of activity is desirable. The former embodiment is discussed first.

In one embodiment, at least one of the MSH1 polypeptides or an allele thereof, of the invention is expressed in a higher organism, e.g., a plant. A nucleotide sequence of the present invention is inserted into an expression cassette, which is then preferably stably integrated in the genome of said plant. In another preferred embodiment, the nucleotide sequence is included in a non-pathogenic self-replicating virus. Plants transformed in accordance with the present invention may be monocots or dicots and include, but are not limited to, maize, wheat, barley, rye, millet, chickpea, lentil, flax, olive, fig almond, pistachio, walnut, beet, parsnip, citrus fruits, including, but not limited to, orange, lemon, lime, grapefruit, tangerine, minneola, and tangelo, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, and woody plants such as coniferous and deciduous trees.

Once a desired nucleotide sequence has been transformed into a particular plant species, it may be propagated in that species or moved into other varieties of the same species, particularly including commercial varieties, using traditional breeding techniques.

Accordingly, the present invention provides a method for producing a transfected plant cell or transgenic plant comprising the steps of a) transfecting a plant cell to contain a heterologous DNA segment encoding a protein and derived from an MSH1 polynucleotide not native to said cell (the polynucleotide indeed could be native but the expression pattern could be developmentally altered, still leading to the preferred effect); wherein said polynucleotide is operably linked to a promoter that can be used effectively for expression of transgenic proteins; b) optionally growing and maintaining said cell under conditions whereby a transgenic plant is regenerated therefrom; c) optionally growing said transgenic plant under conditions whereby said DNA is expressed, whereby the total amount of MSH1 polypeptide in said plant is altered. In a preferred embodiment, the method further comprises the step of obtaining and growing additional generations of descendants of said transgenic plant which comprise said heterologous DNA segment wherein said heterologous DNA segment is expressed. As used herein, “heterologous DNA”, or, in some cases, “transgene” refers to foreign genes or polynucleotides, or additional, or modified versions of native or endogenous genes or polynucleotides (perhaps driven by different promoters) in order to alter the traits of a plant in a specific manner.

The invention also provides plant cells which comprise heterologous DNA encoding an MSH1 polypeptide. In a preferred embodiment, the transgenic plant cell is a propagation material of a transgenic plant. The present invention also provides a transfected host cell comprising a host cell transfected with a construct comprising a promoter, enhancer or intron polynucleotide from an MSH1 polynucleotide, and a polynucleotide encoding a reporter protein.

The present invention also provides a method of preparing a transgenic plant comprising: a) producing a transfected plant cell having a transgene encoding an MSH1 polypeptide whereby MSH1 expression in said plant cell is altered; and b) growing a transgenic plant from the transfected plant cell wherein the MSH1 transgene is expressed in the transgenic plant. The expression of the transgene includes an increase or decrease in MSH1 expression. In some embodiments, the expression of the transgene produces an RNA that may interfere with a native MSH1 gene such that the expression of the native gene is either eliminated or reduced, resulting in a useful outcome.

The invention also provides a transgenic plant containing heterologous DNA which encodes an MSH1 polypeptide that is expressed in plant tissue, including expression in a vector introduced into the plant.

The present invention also provides an isolated polynucleotide which includes a transcription control element operably linked to a polynucleotide that encodes the MSH1 gene in plant tissue. In preferred embodiment, the transcription control element is the promoter native to an MSH1 gene.

In some embodiments, a nucleotide sequence of this invention is expressed in transgenic plants, thus causing the biosynthesis of the corresponding MSH1 polypeptide in the transgenic plants. In this way, transgenic plants with characteristics related to MSH1 expression are generated. For their expression in transgenic plants, the nucleotide sequences of the invention may require modification and optimization. Although preferred gene sequences may be adequately expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17.477-498 (1989)). All changes required to be made within the nucleotide sequences such as those described above are made using well known techniques of site directed mutagenesis, PCR, and synthetic gene construction using the methods described in the published patent applications EP 0 385 962 (to Monsanto), EP 0 359 472 (to Lubrizol), and WO 93/07278 (to Ciba-Geigy).

For efficient initiation of translation, sequences adjacent to the initiating methionine may require modification. For example, they can be modified by the inclusion of sequences known to be effective in plants. Joshi has suggested an appropriate consensus for plants (NAR 15: 6643-6653 (1987)) and Clontech suggests a further consensus translation initiator (1993/1994 catalog, page 210). These consensuses are suitable for use with the nucleotide sequences of this invention. The sequences are incorporated into constructions comprising the nucleotide sequences, up to and including the ATG (while leaving the second amino acid unmodified), or alternatively up to and including the GTC subsequent to the ATG (with the possibility of modifying the second amino acid of the transgene).

Expression of the nucleotide sequences in transgenic plants is driven by transcription control elements shown to be functional in plants. Transformation of plants with a polynucleotide under the control of these regulatory elements provides for controlled expression in the transformed plant. Such transcription control elements have been described above. In addition to the selection of a suitable initiator of transcription, constructions for expression of MSH1 polypeptide in plants require an appropriate transcription terminator to be attached downstream of the heterologous nucleotide sequence. Several such terminators are available and known in the art (e.g. tml from CaMV, E9 from rbcS). Any available terminator known to function in plants can be used in the context of this invention.

Numerous other sequences can be incorporated into expression cassettes described in this invention. These include sequences which have been shown to enhance expression such as intron sequences (e.g. from Adhl and bronze1) and viral leader sequences (e.g. from TMV, MCMV and AMV).

It may be preferable to target expression of the nucleotide sequences of the present invention to different cellular localizations in the plant. In some cases, localization in the cytosol may be desirable, whereas in other cases, localization in some subcellular organelle may be preferred. Subcellular localization of heterologous DNA encoded polypeptides is undertaken using techniques well known in the art. Typically, the DNA encoding the target peptide from a known organelle-targeted gene product is manipulated and fused upstream of the nucleotide sequence. Many such target sequences are known for the chloroplast and their functioning in heterologous constructions has been shown. The expression of the nucleotide sequences of the present invention is also targeted to the endoplasmic reticulum or to the vacuoles of the host cells. Techniques to achieve this are well-known in the art.

Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construction of interest may be preferred. In the case of direct gene transfer, transformation with a single DNA species or co-transformation can be used (Schocher et al. Biotechnology 4: 1093-1096 (1986)). For both direct gene transfer and Agrobacterium-mediated transfer, transformation is usually (but not necessarily) undertaken with a selectable marker which may provide resistance to an antibiotic (kanamycin, hygromycin or methotrexate) or a herbicide (basta). The choice of selectable marker is not, however, critical to the invention.

In another preferred embodiment, a nucleotide sequence of the present invention is directly transformed into the plastid genome. A major advantage of plastid transformation is that plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Natl. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-polypeptide antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19: 4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant polypeptide. In a preferred embodiment, a nucleotide sequence of the present invention is inserted into a plastid targeting vector and transformed into the plastid genome of a desired plant host. Plants homoplastic for plastid genomes containing a nucleotide sequence of the present invention are obtained, and are preferentially capable of high expression of the nucleotide sequence.

In some embodiments, a reduction or suppression of MSH1 polypeptide activity is desired. In some embodiments, a reduction of MSH1 polypeptide activity may be obtained by introducing into plants an antisense construct based on an MSH1 cDNA or gene sequence. For antisense suppression, an MSH1 cDNA or gene is arranged in reverse orientation relative to the promoter sequence in the transformation vector. The introduced sequence need not be a full length MSH1 cDNA or gene, and need not be exactly homologous to the native MSH1 cDNA or gene found in the plant type to be transformed. Generally, however, where the introduced sequence is of shorter length, a higher degree of homology to the native MSH1 sequence will be needed for effective antisense suppression. The introduced antisense sequence in the vector generally will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of mRNA molecules transcribed from the endogenous MSH1 gene in the plant cell. Although the exact mechanism by which antisense RNA molecules interfere with gene expression has not been elucidated, it is believed that antisense RNA molecules bind to the endogenous mRNA molecules and thereby inhibit translation of the endogenous mRNA. The production and use of anti-sense constructs are disclosed, for instance, in U.S. Pat. No. 5,773,692 (using constructs encoding anti-sense RNA for chlorophyll a/b binding protein to reduce plant chlorophyll content), and U.S. Pat. No. 5,741,684 (regulating the fertility of pollen in various plants through the use of anti-sense RNA to genes involved in pollen development or function).

Suppression of endogenous MSH1 gene expression can also be achieved using ribozymes. Ribozymes are synthetic RNA molecules that possess highly specific endoribonuclease activity. The production and use of ribozymes are disclosed in U.S. Pat. No. 4,987,071 to Cech and U.S. Pat. No. 5,543,508 to Haselhoff. Inclusion of ribozyme sequences within antisense RNAs may be used to confer RNA cleaving activity on the antisense RNA, such that endogenous mRNA molecules that bind to the antisense RNA are cleaved, leading to an enhanced antisense inhibition of endogenous gene expression.

Constructs in which an MSH1 cDNA or gene (or variants thereof) are over-expressed may also be used to obtain co-suppression of the endogenous MSH1 gene in the manner described in U.S. Pat. No. 5,231,021 to Jorgensen. Such co-suppression (also termed sense suppression) does not require that the entire MSH1 cDNA or gene be introduced into the plant cells, nor does it require that the introduced sequence be exactly identical to the endogenous MSH1 gene. However, as with antisense suppression, the suppressive efficiency will be enhanced as (1) the introduced sequence is lengthened and (2) the sequence similarity between the introduced sequence and the endogenous MSH1 gene is increased.

Constructs expressing an untranslatable form of an MSH1 mRNA may also be used to suppress the expression of endogenous MSH1 activity. Methods for producing such constructs are described in U.S. Pat. No. 5,583,021 to Dougherty et al. such constructs may be prepared by introducing a premature stop codon into an MSH1 ORF.

Polynucleotides of the present invention may also be used to specifically suppress gene expression by methods such as RNA interference (RNAi), which may also include cosuppression and quelling. This and other techniques of gene suppression are well known in the art. A review of this technique is found in Science 288:1370-1372, 2000. Traditional methods of gene suppression, employing antisense RNA or DNA, operate by binding to the reverse sequence of a gene of interest such that binding interferes with subsequent cellular processes and thereby blocks synthesis of the corresponding protein. RNAi also operates on a post-transcriptional level and is sequence specific, but suppresses gene expression far more efficiently

Studies have demonstrated that one or more ribonucleases specifically bind to and cleave double-stranded RNA into short fragments. The ribonuclease(s) remains associated with these fragments, which in turn specifically bind to complementary mRNA, i.e. specifically bind to the transcribed mRNA strand for the gene of interest. The mRNA for the gene is also degraded by the ribonuclease(s) into short fragments, thereby obviating translation and expression of the gene. Additionally, an RNA polymerase may act to facilitate the synthesis of numerous copies of the short fragments, which exponentially increases the efficiency of the system. A unique feature of this gene suppression pathway is that silencing is not limited to the cells where it is initiated. The gene-silencing effects may be disseminated to other parts of an organism and even transmitted through the germ line to several generations.

Specifically, polynucleotides of the present invention are useful for generating gene constructs for silencing specific genes. Polynucleotides of the present invention may be used to generate genetic constructs that encode a single self-complementary RNA sequence specific for one or more genes of interest. Genetic constructs and/or gene-specific self-complementary RNA sequences may be delivered by any conventional method known in the art. Within genetic constructs, sense and antisense sequences flank an intron sequence arranged in proper splicing orientation making use of donor and acceptor splicing sites. Alternative methods may employ spacer sequences of various lengths rather than discrete intron sequences to create an operable and efficient construct. During post-transcriptional processing of the gene construct product, intron sequences are spliced-out, allowing sense and antisense sequences, as well as splice junction sequences, to bind forming double-stranded RNA. Select ribonucleases bind to and cleave the double-stranded RNA, thereby initiating the cascade of events leading to degradation of specific mRNA gene sequences, and silencing specific genes. Alternatively, rather than using a gene construct to express the self-complementary RNA sequences, the gene-specific double-stranded RNA segments are delivered to one or more targeted areas to be internalized into the cell cytoplasm to exert a gene silencing effect.

Using this cellular pathway of gene suppression, gene function may be studied and high-throughput screening of sequences may be employed to discover sequences affecting gene expression. Additionally, genetically modified plants may be generated.

Finally, dominant negative mutant forms of the disclosed sequences may be used to block endogenous MSH1 activity. Such mutants require the production of mutated forms of the MSH1 protein that interact with the same molecules as MSH1 but do not have MSH1 activity.

E. MSH1 Antibodies

The present invention also includes isolated antibodies capable of selectively binding to an MSH1 polypeptide of the present invention or to a mimetope thereof. Such antibodies are also referred to herein as anti-MSH1 antibodies. Particularly preferred antibodies of this embodiment include anti-A. thaliana MSH1 antibodies.

Isolated antibodies are antibodies that have been removed from their natural milieu. The term “isolated” does not refer to the state of purity of such antibodies. As such, isolated antibodies can include anti-sera containing such antibodies, or antibodies that have been purified to varying degrees.

As used herein, the term “selectively binds to” refers to the ability of antibodies of the present invention to preferentially bind to specified polypeptides and mimetopes thereof of the present invention. Binding can be measured using a variety of methods known to those skilled in the art including immunoblot assays, immunoprecipitation assays, radioimmunoassays, enzyme immunoassays (e.g., ELISA), immunofluorescent antibody assays and immunoelectron microscopy; see, for example, Sambrook et al., ibid., and Harlow & Lane, 1990, ibid.

Antibodies of the present invention can be either polyclonal or monoclonal antibodies. Antibodies of the present invention include functional equivalents such as antibody fragments and genetically-engineered antibodies, including single chain antibodies, that are capable of selectively binding to at least one of the epitopes of the polypeptide or mimetope used to obtain the antibodies. Antibodies of the present invention also include chimeric antibodies that can bind to more than one epitope. Preferred antibodies are raised in response to polypeptides, or mimetopes thereof, that are encoded, at least in part, by a polynucleotide of the present invention.

A preferred method to produce antibodies of the present invention includes (a) administering to an animal an effective amount of a polypeptide or mimetope thereof of the present invention to produce the antibodies and (b) recovering the antibodies. In another method, antibodies of the present invention are produced recombinantly using techniques as heretofore disclosed to produce MSH1 polypeptides of the present invention. Antibodies raised against defined polypeptides or mimetopes can be advantageous because such antibodies are not substantially contaminated with antibodies against other substances that might otherwise cause interference in a diagnostic assay.

Antibodies of the present invention have a variety of potential uses that are within the scope of the present invention. For example, such antibodies can be used (a) as reagents in assays to detect expression of MSH1 by plant, (b) as tools to screen expression libraries and/or to recover desired polypeptides of the present invention from a mixture of polypeptides and other contaminants and/or (c) to modulate the function of an MSH1 polypeptide (e.g., increase or decrease the level or activity of an MSH1 polypeptide). Antibodies of the present invention can be used to target cytotoxic, therapeutic or imaging agents to subjects in order to deliver therapeutic agents or localize imaging agents to RA-affected organs or tissues. Targeting can be accomplished by conjugating (i.e., stably joining) such antibodies to the therapeutic or imaging agents using techniques known to those skilled in the art.

F. Methods for Effecting Mitochondrial Ectopic Recombination and Identification of Mutants Arising from Mitochondrial Ectopic Recombination

In one embodiment, the invention provides a method to identify a compound capable of inhibiting MSH1 activity (e.g., effecting ectopic recombination) of a plant, said method comprising contacting an isolated plant MSH1 nucleic acid molecule with a putative inhibitory compound which, in the absence of said compound, said plant MSH1 nucleic acid molecule has the activity of suppressing ectopic recombination; and determining if said putative inhibitory compound inhibits said activity. The present invention also comprises a method for effecting mitochondrial ectopic recombination comprising providing a plant, and suppressing expression of an MSH1-homologous gene in the plant. A preferred inhibitory compound is an RNA molecule having RNAi activity.

The invention further provides a method for identification of mutants arising from mitochondrial ectopic recombination comprising providing a plant, and suppressing expression of an MSH1-homologous gene in the plant, and detecting an aberrant phenotype, whereby a mutant is identified. A preferred aberrant phenotype includes cytoplasmic male sterility. Cytoplasmic male sterility is a plant trait that facilitates a cost-effective strategy for the production of proprietary hybrids. Hybrid seed is valued for producing higher yields and more uniform crop stands. Hybrids are important in a large number of horticultural and agronomic crops including corn, sorghum, rice, wheat, tomato, rape, sunflower, carrot, onion, sugar beet, to name few. Cytoplasmic male sterility (CMS) mutations arise as the consequence of ectopic recombination events that produce novel expressed DNA sequences within the mitochondrial genome. This is well documented in the scientific literature. The present invention also includes mutants identified the method of the invention.

### EXAMPLES

### Example 1

### Identification of the AtMSH1 Gene

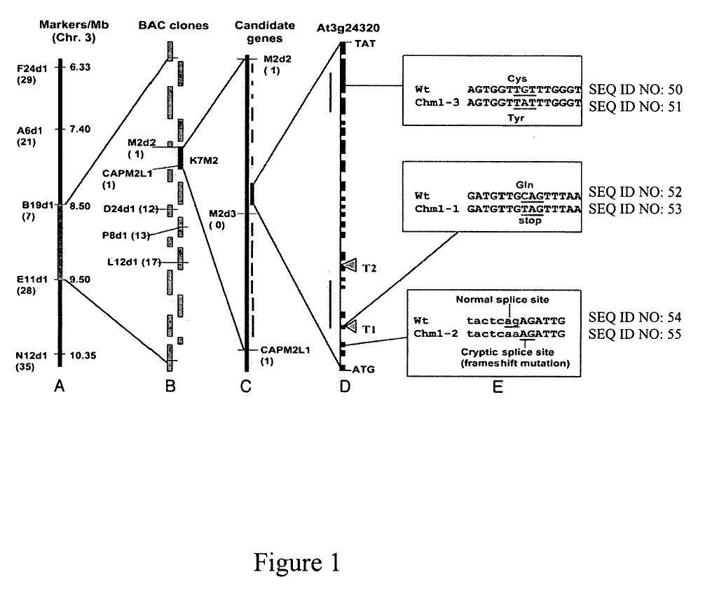

A. Gene mapping, cloning, and sequence analysis. A map-based cloning strategy for the isolation of the CHM locus involved the design of PCR-based co-dominant markers, using the Cereon Arabidopsis polymorphism collection (Jander, et al., ibid.) to distinguish between the Col-0 and Landsburg erecta ecotypes used in the F₂ mapping populations. The markers were designed in a 5-Mb region of Chromosome III based on information from the classical mapping experiments of CHM (Martinez-Zapater, et al., ibid.; Redei, ibid.). The primer sequences for markers are available upon request. The F₂ mapping population was derived from a cross between the chm1-1 mutant line and Landsburg erecta ecotype (pollen donor). A segregating sub-population of 172 variegated plants was analyzed. Genomic DNA purification was conducted according to Li and Chory, ibid.. DNA gel blot analysis was conducted using the protocol of Sambrook et al., ibid.. High resolution mapping of the CHM locus on Arabidopsis Chromosome III delimited the gene to an 80-kb interval as shown in FIG. 1.

DNA sequencing of the candidate locus in chm1-1, chm1-2 and chm1-3 mutants (Kanazawa, et al.. ibid.) was conducted in a Beckman/Coulter CEQ2000XL 8-capillary DNA sequencer. Two independent PCR samples for each mutant were sequenced. The 5′ RACE analysis was done with the GeneRacer® Kit (Invitrogen, Carlsbad, Calif.). Mutants chm1-1 and chm1-2 were obtained from the Arabidopsis Biological Resource Center, and mutant chm1-3 was provided by a colleague. Sequence analysis of the interval revealed a gene candidate with similarity in sequence features to the MutS gene of E. coli (FIG. 2). MutS is a component of the E. coli mismatch repair and DNA recombination apparatus (Marti, et al., ibid.). The gene, comprised of 22 exons, was predicted to encode a 43-amino acid mitochondrial targeting presequence with mitochondrial targeting values of 0.916 (MitoProt), 0.943 (Predator) and 0.856 (TargetP). RNA gel blots showed that the transcript derived from this gene was 3.5 kb in size and the encoded protein 1118 amino acids in length, predicting a 124-kDa polypeptide.

The two sequence-indexed T-DNA insertion mutants were identified on the SiGnAL (Salk Institute Genomic Analysis Laboratory) website (Accessions SALK041951 (SEQ ID NO:5) and SALK046763 (SEQ ID NO:4)), and seed for the mutants obtained from the Arabidopsis Biological Resource Center (ABRC). The T-DNA insertion positions were confirmed by DNA sequencing of the insertion junctions. The first insertion was located within the fourth exon and the second within the eighth intron. Analysis of the T-DNA mutants (T3 generation) revealed mild green-white leaf variegation, growing more intense in the following selfed generation. Variegated plants having a green-white variegation phenotype carried a mitochondrial genome rearrangement similar to that observed in the mutants chm1-1 and chm1-2. A population of 60 T4 plants segregating for one of the T-DNA (SALK041951) mutations (16 wildtype, 31 hemizygous, 13 homozygous for the T-DNA) showed co-segregation of the T-DNA with the mitochondrial shifting phenotype. Of the 13 progeny homozygous for the T-DNA insertion, eight were variegated and the remaining five showed no obvious variegation phenotype. Incomplete penetrance of the variegation phenotype is characteristic of chm1-1 and chm1-2 mutants (Redei, ibid.).

DNA gel blot hybridization analysis of mitochondrial genome configuration using the mitochondrial atp9-rp116 junction sequence associated with substoichometric shifting (Sakamoto, et al., ibid.) as probe. Total genomic DNA was digested with BamHI, subjected to gel electrophoresis, blotted and probed. Lane Wt designates wildtype ecotype Columbia-0, lane C1 designates mutant chm1-1, and TI and T2 designate two sister lines containing the T-DNA1 insertion mutation. DNA band pattern changes previously associated with substoichiometric shifting were noted (Martinez-Zapater, et al., ibid.).

Cosegregation analysis of mitochondrial substoichiometric shifting with the T-DNA1 insertion mutation. A three-primer PCR-based assay to detect substoichiometric shifting (Sakamoto, et al., ibid.) was used to assay wildtype Col-0 (Wt), mutant chm1-1 (C1) and individual plants segregating for presence of the T-DNA insertion within the candidate CHM locus.

All progeny homozygous for the T-DNA insertion mutation showed the mitochondrial shifting phenotype. None of the segregants hemizygous for, or lacking, the T-DNA mutation showed evidence of variegation. The hemizygous plants showed no mitochondrial shifting. Similar co-segregation results were obtained for the second TDNA (SALK046763) mutation as well.

To test further the possibility that the identified MutS-homologous sequence was CHM, we sequenced the chm1-1 and chm1-2 alleles of the gene. The chm1-1 line had a single nucleotide (C-T) substitution that gave rise to a premature stop codon within the fourth exon (FIG. 1E). The chm1-2 mutant had a single nucleotide (G-A) substitution at the intron-exon junction of Exon 2 (FIG. 1E). This substitution resulted in two-nucleotide slippage of the intron splice site, producing a frameshift and premature termination of translation five amino acids beyond the mutation site. Therefore, in both chm1-1 and chm1-2 mutant lines, the CHM candidate locus is predicted to give rise to highly truncated, inactive peptides.

Sequence analysis of the chm1-3 allele, derived from a tissue culture line by Martinez-Zapater et al. (Martinez-Zapater, et al., ibid.), revealed an amino acid substitution (Cys-Tyr) within the ATP binding domain (FIG. 1E). The mutant phenotype in this case may be due to the substitution of a bulkier amino acid within a site essential for protein function.

B. The CHM candidate has features of a mismatch repair component. The MutS-homologous gene identified as a candidate for CHM displayed several features characteristic of a mismatch repair component. These features included an ATP-binding domain (aa 761-946) comprised of four well conserved motifs designated M1-M4 (Obmolova, et al., ibid.; FIG. 2B). In addition to ATPase function, this domain appears to be involved in dimerization of the protein (Obmolova, et al.; Lamers, et al.), although this has not yet been demonstrated for mitochondrial MutS homologs. A DNA binding domain (aa 129-206) was also identified (FIGS. 1, 2) to contain the aromatic doublet (FY) motif that is characteristic of this domain in MutS and MutS-like proteins (FIG. 2A). This doublet was shown to be essential for mismatch recognition and specific DNA binding activity (33, 34). We were unable to detect three other conserved domains characteristic of MutS. A connector domain, involved in inter-domain interactions, a core domain and a clamp domain, involved in nonspecific double-strand DNA binding, did not appear to be well conserved. The CHM candidate protein likely localizes to mitochondria. To confirm that the MutS-like protein localized to the mitochondrion, we conducted RACE-PCR and discovered a transcript start site at 578 residues upstream to the site predicted in the Munich Information Center for Protein Sequences (MIPS) database (Schoof, et al.) and in GenBank (Accession AP000382). No start site was observed by RACE analysis at the point predicted by the MIPS database, and three clustered transcription start sites were detected at the upstream site. The confirmed start site added 102 amino acids to the predicted protein product and permitted the identification of a mitochondrial targeting presequence that was omitted from the previous database entries. The sequence was annotated based on cDNA sequence analysis and is available as GenBank Accession AY191303.

### Example 2

### Plant Transformation and Biolistic Delivery

The amino acid sequence of AtMSH1 was analyzed with MitoProt (Claros & Vincens (1996) Eur. J. Biochem. 241, 779-786), and the first 213 nucleotides of the gene were PCR amplified with the primers

| | |
|---|---|
| MSHtranspFor | |
| 5′GGCCATGGTGTGAATTGCATAGTCGTCG3′ | (SEQ ID NO:48) |
| and | |
| MSHtranspRev | |
| 5′GGCCATGGAAA CATCACTTGACGTCTTC3′. | (SEQ ID NO:49) |

PCR products were ligated to the Pgem®-T Easy Vector System (Promega) and digested with NcoI to release the insert. Insert fragments were ligated to the pCAMBIA 1302 vector at the NcoI site that resides at the start of gfp. This vector utilizes the CaMV 35S promotor. Bombardment experiments used 4-week-old leaves of Arabidopsis (Col-0) with tungsten particles and the Biolistic PDS-1000/He system (Bio-Rad). Particles were bombarded into Arabidopsis leaves using 900-psi rupture discs under a vacuum of 900-psi (1 psi=6.9 kPa). After the bombardment, Arabidopsis leaves were allowed to recover for 18-22 h on Murashige and Skoog media plates at 22° C. in 16 h daylight. Localization of GFP expression was conducted by confocal laser scanning microscopy with Bio-Rad 1024 MRC-ES using 488 nm excitation and two-channel measurement of emission, 522 nm (green/GFP) and 680 nm (red/chlorophyll). Mitochondria were identified by their characteristic movement and rapid inter-conversions from small round to highly elongated, shapes. Plastids located in the cells emit red autofluorescence. Positive controls for mitochondrial (F1-ATPase gamma subunit provided by Dr. D. Stern) and chloroplast (Rubisco Pea/SSU/TPSS, provided by Dr. L. Alison) targeting were included with each experiment.

### Example 3

### Identification of Homologs

Homologs were identified by BLAST search using the tblastn program against the est_others database. The MSH1 protein sequence was used as the Query sequence. The search was done using the BLOSUM62 matrix, word size of 3 and low complexity filter.

What is claimed is:

1. An isolated nucleic acid molecule selected from the group consisting of:
(a) a nucleic acid molecule comprising a nucleic acid sequence selected from the group consisting of SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and SEQ ID NO:45;
(b) a nucleic acid molecule comprising at least a portion of any of said nucleic acid molecules of (a);
(c) a complement of a of a nucleic acid molecule of (a) or (b); and
(d) a nucleic acid molecule comprising an allelic variant of a nucleic acid molecule comprising any of said nucleic acid sequences.

2. The nucleic acid molecule of claim 1, wherein said nucleic acid molecule is a plant nucleic acid molecule.

3. The nucleic acid molecule of claim 1, wherein said nucleic acid molecule is selected from the group consisting of Arabadopsis, Oryza, Glycine, Hordeum, Zea, Medicago, Allium, Citrus, Solanum, Sorghum, Saccharum, Nicotiana, Lycopersicon, Triticum, Zinnia, and Phaseolus nucleic acid molecules.

4. The nucleic acid molecule of claim 1, wherein said nucleic acid molecule is selected from the group consisting of: a nucleic acid molecule comprising a nucleic acid sequence that encodes a protein having an amino acid sequence selected from the group consisting of SEQ ID NO:3, SEQ ID NO:7, SEQ ID NO.:9, SEQ ID NO.:12, SEQ ID NO.:15, SEQ ID NO:17, SEQ ID NO.:19, SEQ ID NO.:22,SEQ ID NO.:24, SEQ ID NO.:26, SEQ ID NO.:31,SEQ ID NO:33, SEQ ID NO.:35, SEQ ID NO.:40, SEQ ID NO.:42, SEQ ID NO:44, SEQ ID NO:47, and SEQ ID NO:65; and a nucleic acid molecule comprising an allelic variant of a nucleic acid molecule encoding a protein having any of said amino acid sequences.

5. An isolated protein encoded by a plant MSH1 nucleic acid molecule that hybridizes to the complement of a nucleic acid molecule having a nucleic acid sequence selected from the group consisting of SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and SEQ ID NO:45 under stringent hybridization conditions.

6. An isolated protein comprising a plant MSH1 protein.

7. The protein of claim 5, wherein said protein comprises an amino acid sequence selected from the group consisting of SEQ ID NO:3, SEQ ID NO:7, SEQ ID NO.:9, SEQ ID NO.:12, SEQ ID NO.:15, SEQ ID NO:17, SEQ ID NO.:19, SEQ ID NO.:22,SEQ ID NO.:24, SEQ ID NO.:26, SEQ ID NO.:31,SEQ ID NO:33, SEQ ID NO.:35, SEQ ID NO.:40, SEQ ID NO.:42, SEQ ID NO:44, SEQ ID NO:47 and SEQ ID NO:65.

8. The protein of claim 5, wherein said protein comprises at least a portion of an amino acid sequence selected from the group consisting of SEQ ID NO:3, SEQ ID NO:7, SEQ ID NO.:9, SEQ ID NO.:12, SEQ ID NO.:15, SEQ ID NO:17, SEQ ID NO.:19, SEQ ID NO.:22,SEQ ID NO.:24, SEQ ID NO.:26, SEQ ID NO.:31,SEQ ID NO:33, SEQ ID NO.:35, SEQ ID NO.:40, SEQ ID NO.:42, SEQ ID NO:44, SEQ ID NO:47 and SEQ ID NO:65.

9. A method to identify a compound capable of inhibiting MSH1 activity of a plant, said method comprising:
(a) contacting an isolated plant MSH1 nucleic acid molecule selected from the group consisting of SEQ ID NO:1, SEQ ID NO:6, SEQ ID. NO:8, SEQ ID NO:10, SEQ ID NO:11, SEQ ID NO:13, SEQ ID NO:14, SEQ ID NO:16, SEQ ID NO:18, SEQ ID NO:21, SEQ ID NO:23, SEQ ID NO:25, SEQ ID NO:27, SEQ ID NO:28, SEQ ID NO:29, SEQ ID. NO:32, SEQ ID NO:34, SEQ ID NO:36, SEQ ID NO:37, SEQ ID NO:38, SEQ ID NO:41, SEQ ID NO:43, and SEQ ID NO:45 with a putative inhibitory compound which, in the absence of said compound, said plant MSH1 nucleic acid molecule has the activity of suppressing ectopic recombination; and
(b) determining if said putative inhibitory compound inhibits said activity.

10. The method of claim 9, wherein the putative inhibitory compound is a RNA molecule suspected of having RNAi activity.

11. A compound identified by the method of claim 9.

12. A method for identification of plant mutants arising from mitochondrial ectopic recombination comprising
(a) providing a plant,
(b) suppressing expression of an MSH1-homologous gene in the plant, and
(c) detecting an aberrant phenotype,
whereby a plant mutant is identified.

13. The method of claim 12, wherein said suppressing expression of an MSH1-homologous gene in said plant comprises contacting said plant with an compound identified by the method of claim 9.

14. The method of claim 12, wherein said aberrant phenotype is cytoplasmic male sterility.

15. A plant mutant identified by the method of claim 12.


SEQUENCE LISTING (65 sequences)

SEQ ID NO: 1 - DNA, 3673 bp, Arabidopsis thaliana
agaggactgtgagattgtgaattgcatagtcgtcgtcttctggcgggaaaagaagccctagaaaaagggtgaaaggtgaaaactctacttcttcttcttcttcttcttcagagtgtgagagagatgcattggattgctaccagaaacgccgtcgtttcattcccaaaatggcggttcttcttccgctcctcatatcgcacttactcttccctcaaaccctcctccccaattctacttaatagaaggtactctgaggggatatcttgtctcagagatggaaagtctttgaaaagaatcacaacggcttctaagaaagtgaagacgtcaagtgatgttctcactgacaaagatctctctcatttggtttggtggaaggagagattgcagacatgtaagaaaccatctactcttcagcttattgaaaggcttatgtacaccaatttacttggtttggaccctagcttgaggaatggaagtttaaaagatggaaacctcaactgggagatgttgcagtttaagtcaaggtttccacgcgaagttttgctctgcagagtaggagaattttatgaggctattggaatagatgcttgtatacttgttgaatatgctggtctcaatccttttggtggtcttcgatcagatagtattccaaaggctggctgcccaattatgaatcttcgacagactttggatgacctgacacgcaatggttattcagtgtgtattgtggaggaagttcaggggccaacaccagcacgctcccgtaaaggtcgatttatttcagggcatgcacatccaggaagtccttatgtatatgggcttgtcggtgttgaccatgatcttgactttcctgatcctatgcctgttgttgggatatctcgttcagcaagggggtattgtatgatatctattttcgagactatgaaagcatattcgctagatgatggtctaacagaagaagccttagttaccaagctccgcactcgtcgctgtcatcatcttttcttacatgcatcgttgaggcacaatgcatcagggacgtgccgctggggagagtttggggaagggggtctactctggggagaatgcagtagcaggaattttgaatggtttgaaggagatactctttccgagctcttatcaagggtcaaagatgtttatggtcttgatgatgaagtttcctttagaaatgtcaatgtaccttcaaaaaatcggccacgtccgttgcatcttggaacggctacacaaattggtgccttacctactgaaggaataccttgtttgttgaaggtgttacttccatctacgtgcagtggtctgccttctttgtatgttagggatcttcttctgaaccctcctgcttacgatattgctctgaaaattcaagaaacgtgcaagctcatgagcacagtaacatgttcaattccagagtttacctgcgtctcttctgctaagcttgtgaagcttcttgagcaacgggaagccaactacattgagttctgtcgaataaaaaatgtgcttgatgatgtattacatatgcatagacatgctgagcttgtggaaatcctgaaattattgatggatcctacctgggtggctactggtttgaaaattgactttgacacttttgtcaacgaatgtcattgggcgtctgatacaattggtgaaatgatctctttagatgagaatgaaagtcatcagaatgtaagtaaatgtgacaatgtcccgaacgaattcttttatgatatggagtcttcatggcgaggtcgcgttaagggaattcatatagaggaagaaatcactcaagtagaaaaatcagctgaggctttatctttagcagtagctgaggattttcaccctattatatcaagaattaaggccaccactgcttcacttggtggcccgaaaggcgaaatcgcatatgcaagagagcatgagtctgtttggttcaaggggaaacggtttacgccatctatctgggctggtactgcaggggaagaccaaataaaacagctgaaacctgccttagactcgaaaggaaaaaaggttggagaagaatggtttacgaccccaaaggtggaaattgctttagtcagataccatgaagctagtgagaatgcaaaagctcgggtgttggaactgttgcgcgagttatccgttaaattgcaaacaaaaataaatgttcttgtctttgcatctatgcttctggtcatttcaaaagcattattttcccatgcttgtgaagggagaaggcgaaagtgggtttttccaacgcttgtcggattcagtttagatgagggcgcaaaaccattagatggtgccagtcgaatgaagctgacaggcctgtcaccttattggtttgatgtatcttctggaaccgctgttcacaataccgttgacatgcaatcactgtttcttctaactggacctaacggtggtggtaaatcgagtttgctcagatcaatatgcgcagctgctctacttggaatttccggtttaatggttccagctgaatcagcttgtattcctcactttgattccatcatgcttcacatgaaatcatatgacagccctgtagacggaaaaagttctttccaggtagaaatgtcggaaatacgatctattgtaagccaggctacttcgagaagcctagtgcttatagatgagatatgccgagggacagagacagcaaaaggcacctgtatcgctggtagtgtggtagagagtcttgacacaagtggttgtttgggtattgtatctactcatctccatggaatcttcagtttacctcttacagcgaaaaacatcacatataaagcaatgggagccgaaaatgtcgaagggcaaaccaagccaacttggaaattgacagatggagtctgcagagagagtcttgcgtttgaaacagctaagagggaaggtgttcccgagtcagttatccaaagagctgaagctctttacctctcggtctatgcaaaagacgcatcagctgaagttgtcaaacccgaccaaatcataacttcatccaacaatgaccagcagatccaaaaaccagtcagctctgagagaagtttggagaaggacttagcaaaagctatcgtcaaaatctgtgggaaaaagatgattgagcctgaagcaatagaatgtctttcaattggtgctcgtgagcttccacctccatctacagttggttcttcatgcgtgtatgtgatgcggagacccgataagagattgtacattggacagaccgatgatcttgaaggacgaatacgtgcgcatcgagcaaaggaaggactgcaagggtcaagttttctataccttatggttcaaggtaagagcatggcttgtcagttagagactctattgattaatcaactccatgaacaaggctactctctggctaacctagccgatggaaagcaccgtaatttcggaacgtcctcaagcttgagtacatcagacgtagtcagcatcttatagtttgaaacattagctgtgtttgtagttgatcatctctatgtgcaattgaacaagtcagtttgctagaactagagtagattactaagaaaccatgccgtttttcattttgagattttgcaaaacggcatgcagttcgggtaagtcggatgccgcaattaccaattttgggtcagtctgtgtaattgtcgtttca

SEQ ID NO: 2 - DNA, 3673 bp, Arabidopsis thaliana; identical to SEQ ID NO: 1
  CDS (124)..(3480)

SEQ ID NO: 3 - protein, 1118 aa, Arabidopsis thaliana
MHWIATRNAVVSFPKWRFFFRSSYRTYSSLKPSSPILLNRRYSEGISCLRDGKSLKRITTASKKVKTSSDVLTDKDLSHLVWWKERLQTCKKPSTLQLIERLMYTNLLGLDPSLRNGSLKDGNLNWEMLQFKSRFPREVLLCRVGEFYEAIGIDACILVEYAGLNPFGGLRSDSIPKAGCPIMNLRQTLDDLTRNGYSVCIVEEVQGPTPARSRKGRFISGHAHPGSPYVYGLVGVDHDLDFPDPMPVVGISRSARGYCMISIFETMKAYSLDDGLTEEALVTKLRTRRCHHLFLHASLRHNASGTCRWGEFGEGGLLWGECSSRNFEWFEGDTLSELLSRVKDVYGLDDEVSFRNVNVPSKNRPRPLHLGTATQIGALPTEGIPCLLKVLLPSTCSGLPSLYVRDLLLNPPAYDIALKIQETCKLMSTVTCSIPEFTCVSSAKLVKLLEQREANYIEFCRIKNVLDDVLHMHRHAELVEILKLLMDPTWVATGLKIDFDTFVNECHWASDTIGEMISLDENESHQNVSKCDNVPNEFFYDMESSWRGRVKGIHIEEEITQVEKSAEALSLAVAEDFHPIISRIKATTASLGGPKGEIAYAREHESVWFKGKRFTPSIWAGTAGEDQIKQLKPALDSKGKKVGEEWFTTPKVEIALVRYHEASENAKARVLELLRELSVKLQTKINVLVFASMLLVISKALFSHACEGRRRKWVFPTLVGFSLDEGAKPLDGASRMKLTGLSPYWFDVSSGTAVHNTVDMQSLFLLTGPNGGGKSSLLRSICAAALLGISGLMVPAESACIPHFDSIMLHMKSYDSPVDGKSSFQVEMSEIRSIVSQATSRSLVLIDEICRGTETAKGTCIAGSVVESLDTSGCLGIVSTHLHGIFSLPLTAKNITYKAMGAENVEGQTKPTWKLTDGVCRESLAFETAKREGVPESVIQRAEALYLSVYAKDASAEVVKPDQIITSSNNDQQIQKPVSSERSLEKDLAKAIVKICGKKMIEPEAIECLSIGARELPPPSTVGSSCVYVMRRPDKRLYIGQTDDLEGRIRAHRAKEGLQGSSFLYLMVQGKSMACQLETLLINQLHEQGYSLANLADGKHRNFGTSSSLSTSDVVSIL

SEQ ID NO: 4 - DNA, 80393 bp, Arabidopsis thaliana
aagcttgcatctctcaagaactctgacaatcagtcaaagggattttctctgagccttatcagcaaacttgaggtaagatcatgtttctccaaactcaacactttcttctcgattttacaattccatcataaacaaaggtcttctctgttccttctccaggtcattgttgtcagtcttatttaatctctcaaatcgaacatttaggatatgtaataacatgattaagccattgtcctagaccaaatacagtttgcaacattgtagatgtaagtgcttttggttttgctcagcgtaacgatttgtctcttgctctactagtcaggaactagcgaactccttggatttagagacccggcaaaacttatcaggcttcatggatgctgttgagaaaatactcgtgcagcaaacccgtgaagaactcaagtccaatgaatcctcccaaaagtgagtaccaagaaccacctcaagagtttgtgcagtttctatctccttattgtttttgtcttgggttgttatctgcaactctttgttgtaattactttggaaattggaattgtatcaatactcttgcttatgtcctcaagtttcctttatatatcaatatccttaaacacattattatcttactctccgccatatataatcagagaactaatgaaataatctccaaataatctccaaatacttctccattctggttaatggaggaaaacaccaaccatatataacttacggagaagtaatctccaaataatctccaaatacttctccattctggttaatggaggaaaacaccaaccatatataacttagtgattacttctccgtaaggtgacaacaaaactcagaggtctctttaaatggaagaagctaaagttgttctttgagtgttttaactgttactttagtcaatttaaggaagtcaatatggcttagtaaatcaattaagaaaaccatttaaaatctaccaagtttagtctaaaagactgttgtctacacttagctaaaccaaaaaacagaaccagaaccagcaccaaataaacctaatgaaatttccattttaaattaaaccacgttcagattgttcttgtcttcacttgagattcttgtgcagtggaacctctttgccttgtagaaagtttttcaatcgtttgcttcaagacaggtttcaatctttctcttacaagagtgacctcgcgcagtttctgattcaaccggttcaagatcgcaggtaaaactctttgatgattttctttcactatttcaagcatatctctaccatataaattttctgcacatctgtagaacttccctccaagatctttcaggtccctctctccgttaataacttcctctgcattcttcgctgcacttgttagaaatcccaccaacatatctagctcgaacatctgatcttcccagttgtacatatctttttcgatatcggttaggttcttctgtgcctggatatcataactgttcacaacccaagtgttgtttagtactgtagacgagaccggtgtctgttcttccttgggaattagcttatagttaggtgtcgctcgttcagtctttctccttttcttcagaggtctctcaggttggtctgattcctttggaggcaacattcctctcttcccctttccctccaacttgatcttatccgactctgtgtcttgggggttatcatcaactttaaccctagttccagcctcatcttctccatgaccgtaggcaatgtctacgaaccgttgataaagattcttgtatttatcgatcaaacgttgaagagacttaacgaattggctgtaatcgattctatgagacttaaagtctttacacaagcaagtgtaaactctcaattccgaccgagttagtctctcctcgatagccttcaataactctgtcaacttctgtttcgcttctaaacggtccttcatctttctctccacatcgtctttaggtttataaactatccgtatacgaggtatcatctttggttcattaagaagaatagggttctcttccggcttctcatcatgatcaatctttcgattaaggttattgtctttaagcctcctaagttctctagtgagttcgggatttctgagtagacgtaccgaagatcgtaaccttgtctgtaccgatgaagactgtgtcttgacaccgttcgtcttcatcaactcatcagcgtcagccatatcttctgaatctcgatagcgtgtgagcgaaacaaacgacttagggtagagtatatataaggtgttttcgttttcctttttttttgaggagattaactatctagatttgctgaaagtacgcagattacttgttttctaaccaatggtaataaatcaatttatttaaattagttttctttcaactgattaaaaacaattaaacgcaaattaaattttgagaatcagtatttgtcttatatataaatttatacatgaaaaatataaaatatttacaacaaaaatcatcatcttatgaagagttcatgaatctcatcaaatctctctgttttctcttgcaacaaaactcacggtgatgtcgtctatacaaatcttgttgctttttatattagaatttgttagattctagtaggctggaagggatcttgaaggatttgtgtttagagatttatgtcattctcattttaggaggtggagaagtcaaaagagaatcaaccatctagcagctcaattggaagtatgtgaagtgattgatgatgacatgttttatagttttgtttctattatatgaagcttgtgccccatctgaaattttctatatttctcttagttctttcctctggctaacatacctaagatactgtatgatatgcatccaacctcaaagttatattttgttgaatcttaaggtgtctgtcataacctatcttaaaactgcaggcaggtaaggttacactcttcagtgctagacacaagatcttcagtccagtgggttgtcgccaaagtgcaagatataattatttctaacaactttgagaaaagattttgtgatgagttcgaaaacaattagttgagccttccctttcagtagcttaagaggctgaatttttctttcgtagctaactcatgtcattgagttctctgagcaagccaattgactgcatgttttgtgatcctatggcaggcacacgttcgaatactacgacaaagatgaaacaattgtgactcatatagctggaggtattgatgcatttttaaaggtctctgatggttggccactgctgaataccccattaaatggaagaagctaaagttagtgttttaactattactatatagtcaatttaggaaagtcaatattgcttagtaaatcaattaagaaaaccatttaaaatctacaaactcaagtctaaaagattgttgtctacagttagctaaaccaaaaaacagaaccaacaccaaaaacaaattaacctaatgaaaattacattctaaattaaaccaggttcagattgttgttgtcttcacttgagattcttgtgcagtggaaggtcatttggattatgtgtaggtttgggatttggctcgagtgatctctacatgtggtgagtttgtaagaaaatgaagttcgggaatcgaaaaaaacgcttcataaacccagtaaagctacaagagatggatgttacaactaaataacacttgcacttgaaaactaaacaaagtttttatacaaagagtgccacaagtggagtggtggtatacacagagtaaaaatgagacaatgtaaacaagagaaggccagtcaagctagacgagtggatctgtcaaaggcctcaatgacttgataaccatatctatgcttacacaaatgaatactgcgggtgacgaggacggaggtgagactgtgcggaagatcaataccggtgcgtttaaaaccccaaatgagacgctgaactacatagttcccaacacgatctttcaccaaattctcaaagtaactatcaaactcagaggcaatcaacgtaagacacctttcatcaagcatatcaagaaacttctgcacagggtaattagagaaggagcctgatgaaagggacacaaagtgaccagagaacttgtttaccctttcttatcaaaggcagctatcaagcaatcattaccaaacatggagagcaaaagtgaaccagcatgatgtataagcagtaataggttcttgttaccaatctcattacgggagagttcaacaaaagttgcaccaacttgaatcaggaacttgaacaaaagttgcaccattctcatattgacttgatttaaatattaacaaaaaattatatctatgttattgtagagaagaaaaaagaaaaacgtatataattaaagattaggaaactcaagtcctttgaagaaaaataagaaataaaaaaacccaagcctaaagtaaaccttgataaatcgtcataccttttctcaacgatactattctataatacatatttaactagataagtaatccgcaccttgtgcagaaaaaaaaattattaaataaatttaacattggattaaatatagaagttattctagtggccaaaatctataatgactttagggtttatatagatatatgtgatatccattatatgatcaataatatattggcaaatatagaacagaacatttatattttattaatatgtttagtatataaaatgatgattcttttgatattataacttattctttataagataaataatattgctaaataattcgcttaacgcattagtaatactatttttatgtatttttgtaaaggtgaaaacgattttatgtaatatatgtttgtgaaaaatttgtagtattgtggatgaatatgatattggtattctatttataggaaaatatgtttttacatatgtaggaaattgtcttttttccttgcaattgttttagaaatttgttacaagatattaatgcgggaaaaatgtaaatttttgtttcggaatatctatagtaaaatatgattttgttcccatattttctaatattttacttatgtattattagttaatgatatacgcagttattctaattggcggcagatttgctatatatacttgattaggggtagttttcttttgtagacacatcaatctgtatgaaatttacaggatgaaaaacactcggccacgaagagccaaaatttaggtttgcgttttgctgtgtgaagagaaaatatgagtcaagtatgggtttgaaattcagtaatttgagaagagttcattgactaagtattgacgtgagtatggtagaatatacaataaaccatatacttacataactaatcataaaaggaattgatagtcttatactacatgcactaagtcactaaccaacctggcttagcatttctaaacacgaatttacaaactgttaagatctcgtggcgtctgacaacatccaataatgacaatggcgatatgaagaaacaaaagcagagggctagaccaagcatgaagttctttaacgttattttttgttaagagactgaagaagaaagaaggattctatggagattggagagagtggttgagctcgggcaagagaataaaccaatgtaccactttagatacaatgttttgagctttctgaagagaaaaaagaagagaaaaacgagactaaacaattattagtttttcatagttatatttaactacaaactgattgttttcggtgtatgtgtataggtaccaaatccattggctttcgagattgaaattaattaagaaactcataattccatacatacatacacaatatatggacgaaatttgagtaaaattcactaagcttctgtccataaaaccttaagtgaaaaagggagatcaatcccaagaatctattgaaaatatgataatcttaagtaaaaaagcaaactcaagaatcatagctttatggatagaacaaattataacgctcttggaagaaaacaagaatcatccgggtcctattctctaccaaaacaaagagtgccaccacacacgaaacttatacacttcgttttcatcgagaatgttcatgtatatgaggaaatcttgagatttagggcaacgtcttgggaatctcctacttataataaattgaaatcaagaaaaaatataactcatgcaatgatcagattccagattcaagatcatataataaatcaaaccctatatcttcatgaaaacaccacaagacatataagaacaatcactacgagagcagtgaatttagtagaaatcaatacaatacataagggtttatcaattgcttgagtaacaatgaacccctaaacaataatgtctagagataaaagatatgtaaatcgaaaagaaataaaccctaagagaatgatcgaaactaattgatatcggaatcatcaaaatcatcctcgaacttgttgaagaaatcgctgtcttcgggcttcatttgaaagggtgaaattcctccaacttcaaactgcatatccatgggcccgaagtcgtcttcatcgatctccatctgcataggcatcgagtatcctggatactccatctcacccagaaagtcacggtacaccatcggcctcatcgggtcttgttcccaatatctatccatcgtttcgcttcctttgcgtgaaaatcaaacaaaacctaaggataaagagaaagagaggtgttctactttttgagggttaggacacccttaagtaatatccgggacattataaaggcccattagtagaggagattggtccattatacagtcaagataaattaagagatttctttctttatgtgtcatcaacaattagtttttaagatttttttctaaaacaaagattttgtattagattatattcaacatatgtagtatctactaaaataagcctttctaataattataatgaccattataaagaaaaaagtatatttatttttatttcttatttttggtattgattaaaatatacatatctaacgtctatataagaaacatatgtgttttacaattatcaaaactgattacttccaacagaaggaaacatacaaatttgttaacatgtattagctgaagctcttcaccagtaaaatcattgcaaagatatatgattcaaaattgggaaaaatcttccctagtagagaacaactcaatgacaatgtgaggacaaaagcagagaagaataaacactaaaagagaaaccttttgtatcatagtatttacaacatttaacgatcataattttgagcattttcaggattgatcaaaatctatatatatatatatgataagagacttcattttgaataatcaaataagatacaaacaaccttaatccgtataatagtcattaatttattagcttgtggtggaggtggtttttctgccacctataggattctcacacgggacaacatgcaagcatgcatattgtcagaaacatactcaagaaagccaaagaaatgaccggcctcgagaatgaccagtatgcaaggacgctatactatcctaagtcctaactcctaatccatagaaaaatactctgaacacgtggtacgtgacttgtcttcttaaacgatacttggctagaaacttgcagtttcgtaatatgtagccgtccaaacttttacttcaaagtcaatctcggttaagaaaacatgttctgtccaccaaggtaataaagatcaagagtacttgatcgactataatttcatcaaatatatcacgacttgacaggctcacctacttatgacggagggctgcgatattggttacaaacacatcctcgctgacgatcattcggttataacctttcaaataatttcaatttcttctaatcctttcaaatagatgacaaaacagtcttttggtgaataaatctttcattcataccaaaaataaataaagtgcaaatgtgcaaatgattgcatataaggaaaataaataaacgtacctagctattttaagtgacactttctaggatttctcagccataatgattccaccgttcttcatggtgctgtgagaaagaggttcattgtagattctatccatgtaggtagaaatttttgttttctcaaatcatcgatatggtttatttagttcctcatgtaagaaaatactttgtttatgaaagttttctctgtggattgatttttcacacagctccgtttcttcggtttcctttggaatgtacatgtaagaagtatgatgtattccattttcaatatgttttatttaacataaaatctagattttcgaccaataaccttttcaagtaaaagaccagaacaagtcaatttagtcatttttcgaacatctaaaacttaaacttatgatgatgttacgactcaatctttttaaaagttcccctattacatcactagagcaatagtttttgtgtaactttagttcctaaaaggagcgagtctaataagtgcataaacaaggtatatactaacagaatttgggaaaaatagtgaaattaaaaaaaaaggtgcacaagaacacaaacatagatagatcactcaaaacaaaacaaaacaaaacaaaacccaaaaaaaaatccaatcaagatttgtatttcaaaagcaaaatcccaataatacttaactggatataaatttcaaaacagcttgaaaatcaaacggctggagggttaggcgcacgaggtactggagatccaggcactccaatggaatcatcatcattatcatggtagatataagcaaaaccaccatgacctgctaaatccattcctcgcatttcatgctgctccgagatcctaagcaaattgagctttttgaggatgaagaagagtgttcccattgtggcactaacccatccaacaatcacaattatttgaaccagttgtgctcccaatagcttccctcctccgcccataaatagcccgtagtgccttcctgggctcgcgccgtaaacctcatttatatacttctcttttgcaaacagtcctacaaatatcaaaccccaagcaccacaccctccgtgtagttgtgcggcctcaagtggatcgtcatattttaagagctctgcgagcttgttgcatccgataaggacgagggaagccacgaagccacatacgatcgctgcccatggatcaaccacagagcaacctgccgttatggccgcaaaccctccgagtaacccgttgcaaacgtcagttacgttccagtggcctgataggagacgttttccgaagagtgtggttagagccgctgtgcatcccgagagtgtagttgtaaccgcggtgcggcctattccactccattggccatagttggaaccagaattgtagggaatgagtatcttggtgaaggaaccagggttgaacccgtaccaaccaaaccagagaaggaaggtccctaagacgacgagtgaggcagagtggcctcgcagagcaatagcatgacccccatcaggaaaccgaccaatccgagggccttcaataagggcaccccataatcctgcgataccaccaaccatgtgaacaacacctgacccagcaaagtcgatggctccagtgccaaacaaacggtcttctgaacggaagggactagcccatccatccggagaccagaaccagtgagagacaactgggtaaacaaacccggtcaaaaaagaagagtatatcaaatacgccacgaacttagtcctctcggcaatggagccgctggtgattccagcggctgcgattgcaaacgcccattggtagaggaagaaggagtaatccgaggtgagagtcggaaagttttgaagaccaaagttgtgtcttccaatgaatccatcggaggattcaccaaaggcaaatgcataaccaaagaggtagtagaagagtcctccggctgcagcatcaaggacattagtgagcatgatgttcatcgtgttcttagctctaacggagccagcacaaagcatagcgaagccgagctgcatcgcaaagacaagataggcagagaagaggaggtacgtgttgtctatagcgtaggctgcatcggtaaacttgttgttaacggaacccaactggccgcaaatgtagtcagccgctgccgtggcatttgggccgagtagggctgagagatcagccgcagagcaagtaatagctcctgacatgttttagagacttgagagaggaaccacacgaataagaaagattgtctcttgtgcagctataaaaagaaaaagaggaggagtttctgattctggcttgaccaatcaaattcgtagttaagtggagagatattcattattaaattattagagatggaatctaacaaatcttataaactacttgcaatatacgagaatcttatacggttttggaaatttagtgattggcatgtactttttatttggtatcaaacgcaatagtttcttattataaaaaaagagagagagggagtttaaattcccttatcacatgcttcttttagcaatctaagcaagctgatggaagcaaatttaacccaaaatatgaatgacgacattataatattgcaaaaagatattaaaaataaaaaatatgatctattcaattgtagcatatgtaagcaaaacatatttgaccaaggaatctaagaagattcccgtagagagttggtcgccgttttcgactcacactgtcacaccacctcacacgaaagggaagctcattcgtgcttttactttcggggaaaaaacgtttacatgtacctgagtatcttctaatttctattttaatagaattatataaacaaaattagtacttctagggtttctagcaaatcaatttgtggcaccttgctagttgtagatcagcaactctttttttgggaatcaggtcaaaattcagcattcttaatacgaaaatcttaaatataatgaacttgtaaagtattaatataattgctgacgaaactgcaaatgataacatggtttagcgggaaccacgttttattagttgaaaatttcacttgcgatcttaaaattcaacaaatcaatggcgtttaatgtgattctatatattagcaaccacaatggtcgaataagaatgagtatagtttatagataaaattgtagcaataagtaatgttccaactatgcctatatgtaaacttaatcattataagacatacttgtaaattcactagtgaatatatacgtacgaaggagaataaataagtttactagattacaaattagctgatggaaaacgaagaagtatacgatttagaatacattaatattgggatttgacactcaggaaaatagacaaaaccaaactttttttttaatctaatgtcgtcgacgaacgataaatattttaattgcaacgggagcttttaaaatcatacagagtaacctgacatcattgattaccacataacacaaattaattatttgataatacgtttaatttactgattaattatagtgcatattatattttagtttaggtcaactgacaattaactataagaactaaaaatttactaaatacgaaaagccacattgggacgttttctgttttatgaaattatgcaacggcgcattgcggcgtctctagctatcctctatgcatatcagactataaaatatttgttcgtgaaattactttttatgttctataatagtgattgagtgtttaataaccaagaacatcataaaaaaaactctatagataatcataataattgcattaacatatttggctatttgccaaaacaatttaactcaatactggttgatttgcaatgctagaggatcttaaagaaccatcttagattctaagtagtaaacaaatatataaatcaagtggtagtggtttagcgattcccattctaggaagcgttgccttgtcggtctagaacaagaaaagattcattacaaaacattgaattatctacatcaattgttatttacctagtcgaaatagtgttatattttttttctttattattagaaaatttggtgaatacactagtacttgacgactataatcactctaagaaattagttgggtggtaaaatgtcggattaccgatagataattaaaataaaataaaactctaagtctaaactatacgggcaagaatatttaactggttcgtaattattttaatattttttcttactacgacccgttaaaggtaatcaaaattcttgtacaatgttctgtctttgtttgggtgacatatgaaatatacgtttgagaatatttaaatacagtttgacgactaatatgcacaaaaagatgatgtgcaaggtataagctactggaattgcatcgtaattccatgattctacaggtctcgaaagtgtatacaaacaaggaatatatgttataaaaatttttgtttagcagaatttgaaataaataaaaaatactattgaaatattaggtgctaaaatttatcaaccacaaaatatgggtcatactcctttgtttaaagttatcgaggacgcaagaatttgatttatattttaattttagtttgtacgattaatagcaacaattgttaggctaataaaacccattaggcttagttcaatagttctaaacaagtgaccagttacatagatgtaatgacatgtcatatttcttctagtattatttattaaccaacatcgttcaagtaaaagataatatctacgtgtacctaagcgtggctagttaactaattggaaaaaatcgctttacgtgtacataagggaatgactaagttctgcttttggtacgaaatggtacacaaatgatttttcagaatttatattattctaagaacaacttttgtgtactccaatttcctaatgccgtcggcagaacaaaaacctaagcaaagttgtatacagtatatgcataactctcctttttggatcaccaaaagttgtatatataaatttctaagaaatagcataaacattagccgaggcatgtaaaattaaaattcaagaaaaaaaaaaaaaacataaactaaaacacttataaacacaaaaagacgataggtcacttaaaacaaaccaaaacatccaaatatttatatttcaaaaccaaaacccaaacaatacttaaacggtaataaattaccaaaaagttgaaaattaaacgcgaggaggagtagctgatcgagggaaaggagatccaggatccactctatgagactcatcatcattatcatggtagatataagcaaagccaccgtgacgtgtcatatccatcccttgcatttcatgctgctccgagatcctaagcagattgagccttttgaggatgaagaagagtgttcccattgtggcactaacccatcctacaatcacaagtatttgaaccaattgtgctcccaacagcttccctcctccgcccataaatagtccatatggccttcccggggtggcgccataaacctcgtttagatacttctctttggcaaacaatcctacgaatatcaacccccacgcgccacaccctccatgtagttgggctgcctcgagtggatcatcatattgtacaagctccgcgagcttgttgcatccgataaggacgacagaagccatgaagccgcacacaatcgctgcccatggctctacgacggagcaacctgcggttatggccgcaaacccaccgagtaacccgttgcaaacgtccgttacgttccagtggcctgataggagacgtttaccaaagagtgtggttagagctgctgtgcatcctgagagtgtggtgttaaccgctgtacggccgattccgctccattggccgtagttggaaccagaattatacggaacgagtatcttagtgaaggaaccggggttgaaaccataccatccaaaccataggaggaaggttcctaagactactagcgaggcagagtggccgcgcagagcaatagcgcgaccacctttctcgaaccgaccacgacgaggaccttcaataagagcaccccataaacctgctatgccaccaaccatgtgaacaacaccggagccagcaaagtcaatggctccggtgctaaacaaacgatcatccgctgaacgaaagggactggcccatccatccggggaccaaaaccagtgagagacaaccgggtaaacaaatccggttaagaaagaagagtatatcaagtaagccacgaactgagtcctctctgcgatcgaaccacttgtgattccagcggccgcgattgcgaacgcccattggtagaggaagaaagagtaatcagctgtgggagtcggaaagtctctaagagcaaagttgtgtcttccaatgaacccttcggaggatcctccaaaggcaaaggcgtaaccaaagagatagtagaagagtcctccggctgcagcgtcaaggacattggtaagcatgatgttcatcgtattcttggctctaacagaaccagcacaaagcatagcgaagccgagctgcatggcgaagacaaggtaggcagagaagaggaggtaggtgttgtctatggcgaaggctgcatcggtgaacttgttgttaacggtgcctaattggccgcaaatgtagtcggccgccgccgtggcgttggggccaagtagggtggcgagatcggccgcagagcatgttattgctcctgacatgtttgagagagctgagagagagaaagagagatacggagaagaaaggttgtctcttgtgtgtctcttaaaaggaagaaggaggagtttagggttgactttagattccggctagaccaattaagttcgtatttaagagagtttatcaattatttaagaagttatgtgacgtaacaacattattgaattagagatgtaatctgccaaatcttcttaataacttgcaaattacgatcctaatcctatacggtttttggtgattaagtgattgccatgtaatgtgtgaataaaaaaaaggtaaattaaaggcttgtataaaaaaattacaaaaataaatttgtgaataaaattgatactagtatttaaagtaataaatcaaagaataattgtcctagctcaaccgtatatgttcaagcaaaccgatcagaacttactacatagatgtcgattttagatcagcagttttatgaaattaagtcaagattcagcactctttatacgacaatcttatgataatcttgcaaaattttaataaagttcataaccaaatattaattaagtttgctgaaggttgaaaccaaaaatgataacatgggttagcgggaaccacgttttatttgctgaaaattgcactttacacccttaactcttagatatgtccaaaattactcagatcatcaacagaagcattaaaaaaaattatcttggttttgtataataatactacatgcatgtataaagtattcaattaggaaaacaaaaaaatgaagacacacgacatatagtttcaagcaagagatatatacaataattcgatccaattgacattgaaacctattaacagttcatgaataattcacgtttataccaaaacaaaaacattcatgtttaagatatatatatatatatatatatatatatattttaagatatttaatgattgaaaaaagacatttatctctcagtttgtgccaagccatgaaaattcaagtcaagtattacttttaggaacatgatgatctcttttgatcaaaaatctattatgatgtatttgaataaatatacttaaaagttgttacaagtgctgatgtttcaccaataggctttttattgtcactaactagttctaaaaactgaaagctttctatatatgtatgtgtgtgtgtgtctgaccttaatcatgttttaagtcaacctaacttaatcattatgggagacacttgtgacttgttatctatatatttactgatgagttgtaaatgagagttgacactcagaaaaatagacaaagcaaactgttttctaaaataatgaacgttaaattatgcacatattttagtagacggtagctttctaaatcgtacaaaataatgtcaagaattgacgtgacatcattgattaccacataacaaatattgattctttagtaatacgtagtaactattgattaattgtagtacatattttttaatttaggtcaactgaaaattataaatgtaaaattgtttactaaatacgaaaagccatagtaagtgatttacattatattggtttatgaaataaaactaaagcaggcttgttatactgtgtaactaggtaagcaccacggctcgacgcaatagcacgttagggagtctatagctcgcctctatgcatatcaaaattcacaatttttgttcgtgaaattctttttttatcctctgatgttagaaaccaaaggcatcataagaaaaattgcgataatcaagacttttgcattaacttttgtgccaaaaaacaattacgccaagagtggtagactttgcattgcttcatgatgactttaaagaaccctgctggattctaagtagtaccattcaaatctattgtttccagctagttctatatattttaaagaatgagttattgactctattagtctctaattattatacttcttagattttttttttttttttggttcaaagttcaacgagtagacattcacagcaagattattttaaatgccgaaagtatttctattaatattcttgcaactaccactttaaatgtaatccaaattattgtacagtataattgaaagtttcgcatttgttgtctttgtttagctgacatatgaaagatacatttaacaagaaaaaaatgcgtgatgcaaatacagtttgactaatatgcagaaaatatgatgtgcaaggaaaagctattggacctgatcgcatttaatatcgtgataacatgatactacagttatccaaactgaatctaacgaatgaataaaaggatttgtattgggtttaggaacatagaatgttattgaattcttcatctaggcaaaattaagttctataaattaatcgcctaccaaatatgggccactcattttttgtttaggaagtctttgcaaagattttaaggcaacgaatggttaggcaaataaagcccatttaggctcgacccgtaaacgtttacaagtggccacttagatttactgacatcaccttatctaaggttattattattcatcagcctaagtattttggttggtaaagtctaaaaggaaatatccacgtatacctaaacgcagctagctaactagttgagaaaaaatcgttcaagttacatataagattactttgaataactttatctttgagatataatttgtttagggaatttatgcttcgattcttgtttcgaaacgatgtaccatactatcatcataactaggtccagaattctagacggcgcccttgtaaactacaagaggaatgatcaagcaagtaaagaggtaatgctgaatccaagactaaattagaaagtaagagatttcattagctttaatgagttcaaagggaatttcacctagtggaagtcggattcataactatttgttccttaaaagcggattcatgtttaacatatttatttgttaaagtaagaataattaatatggctcaaaccgtgttcttgtttttcttttccgtagcactatgccactatctttattatacaaaagcttcggaaattggatgggacacgtttagccaaatccaaatgatagatatcgattccttgttacggaaaaaaaaaaaaaaaaagatatcgattcctttgttttagttttaattttttttgtcgtctacttcatcactattacaagatattaatattttcctatccggtaaaacttactttattaacatacaaatttcgcatttttccgataattgaattccaaacataaaataaaataaactttttggtcaaacacttaatgatatgagcttccaaaaatattaatacacatttaagatctaaataattttggaaagaaagctaaataaatatgaacaaacaagggaaattaactagaaattttttttgttttaaaaaagcggatcactggtttttatttttattttcttttaaaatttatgtttaattttactttctaataatgaactagtgtatgatgatgatccggaagactatccaacatgcagttaagtgttaaaaaaaattttaattaagtttacgagaggctttaataaaagagctaagtacatactggaactattaaaagcaaccaaattgaacagaacactttctattaatctacaagaaactcattaggataatccaagttggaataatctactgtaatatcttaatctcatcaagaagatattttcactacaatatgcaagtgacaaaaaaaatatttgatctattggagtgagttgtgttgtactaccattgacataagcattcttccacatactttcttaaattctactacatcactcttttatgataaaaccaaatgagcttcttacatcacacacaattcataccaaaattatatatgttaaaaccaaaaatctcaaataaatgaaaccacaaactaaactcaaactcgacctaagttgtcggtcaagtttttctctttcattatccggcagataagatattttaaaggtcaagttaacagaagggaatgaccatgttctggaaatagtgcttccttggggcacaagcagcgccaccaaaacttccttgtccttcaaaatccagattccataaaccgtcccaaatagcgttatcttcattcacattagcctcaggtattgttaccggaaccaccgggtaatacccgttactatttggttcacaaccagaagttgttgttgaatgtgtgatctgatgaggtacatataatgctccactttcgccactactgctactgctgaaggtgttatcaccattgttgttgtcgtcatctagtaacgacatgatctttttcatgtcgatttgattgaattgaagcaactgttgttcttgctgcaactccatttgtctttgctgctgaaattgttgcctcttcaagattcggttctttgtcttctccgcactgttagttggagacttcgtcttcttcttgaaatgggttcgccaatagttcttgatttcgttgtctgttcttcccggtaaactacgtgcaatcgtggaccacctacaaatacaaatcatcagacaaaaaaccttagaataaggaaaaagagatgagttacttattagggtaaagctacaattgtttcatattttagtttcgaatgtaatgaaaaaaagttaagagaagaaaattctatttagcatactagaggatgtgaataaacaatcaaagcttaggttaattacctattgccccacttagcatgtaactcaaggataatggtttcttcatgaggagtgatttgtcctctcttgaggtctggtcttaggtagttaacccatcttaacctgcagcttttcccatttctcttcaaccccgcgagcctcgcaacagagttccatcgcccttcaccgtgaagctgcacataatcgatcaaaaggcggtcttcttcagcagtccaaggcccttttctccaaccttcttctaccattccccatcctcctcccatccctccccacaaactcattatttcttcttagtttttttttctttagtgaaagctaaataagagaagtagtgtcttctccttctctaacagtcccaccaatctctttttgtctctctgtcttgtgaaacctaagtgttcctctgttttgtctttgtgttgtgttttgttctgtttttcctcttcccctttcattatttatatatacactcactctagctccttggctccaccactttttttgtttgtttgttttctatagagatatagactagtgacaagacaatctttttacatttttagagttgtgtaccttttagaatcataaatatcatctaaccactaactaaaagaaaattccgtgcaatatacatacctttttccccttaaactacaaaaatagttctacaagctcttaattagcaataattagaatttaatgtaaacacaaaagaattacgagaacttgggtgctctcgttaccccttttcactggtcgttttcataagtctttcgtatgtatcatttcctctgtccattcaactcttgatctccaaaggcatcatcatacacaaattactaaattacccctcttgttggtatatttcttttgagttttaatcatggatgaaaacatgcattgatacattaaaccaaaacttttcttcttttctccaactacattttgagtccttagttgctaaccaaatctgaaagacaaaagtttacaagtttaatttttttaaaaacaattctaagaatctaagcttaatagtctctgtatgggaattacaagtatagaaattaataaaacccgagaaattagaaaagttaacacattgtaaactagtgtcatggtaggttgaaattttttggattaattccaaggagacgtaacaatggtgagttgtggctacttcatcacaatcagtttcaaaaacctatcaatatatatccttatagaaaaatattcttcataacctataacaaaattttcatcttcaaatgaaacagtgaagggtccaatgaaaattttcccttgaagcactatctgatcgtctccctataggctataagcaaatacttcttacgcatatggtggaatgaatcagaagaattttacgagctatcgatcccacatcaaaatttctgaaaaggtcttcccaaagagtatctttcctacataacctcgacagaccaagataaccgttcacttactacacaagttttaaactcattagttttagggtttttgtttttaatatgttaaaccaaaaaaaagaaaaataaattaaatcaaacagaaattatatttgtatatatatatatatatatatatatatataaacgaattcagcctaaatcaaaaactaaacagatccaaagcataatttatttgatttcagttagaattctattgaaaagtttaagaaactaaatcaaatttaaatgttataattattaagccacccaagcttcatcattctcatatacacgaaacgaggatattacagggctattcattttgatccattattttatgcaagattaaagaaataatgttaaggcattaggtaacatggtttaacaaggcatcaaatccaatagaataatccttttaatctaaatttttatctttttgatagggttaagctaaagttatttacaacttgtgttttgttgccacgtatatgatctagaggtttaatgcagggacatatattatgattattataattatttaacttccttaatgaacattttcttagaaagcataattaaaagtaaaagaatatcatggtgctggattattttaaggttatacacattattgaaccactaaggtttctccaagtgtcaaggatatttatacatgcgatcttacactaatctttgttgttgtcgtataagattcgttggattccacataaggagttattacaataaactatagaaacaaaccaaataatatgaaaaagttgatggtgtttgtacgtcaccatgggacaggtggtcttataattctctcctcttagatatttgtaatgtctcttctcatttctttctagatattgtgatcaatccaaaaaaatgaatgtctagcgtttccatctttttccatagtagaaatcaaacaaaaaaatactgcaaaatactaatataacaagaaatatctacgtgaaagatgagttattatatgtcattaatagcgttttatgaaaatcacaaactatgtattatgtctctggtttcatgttgggtacgtatttcagatgaacctaaaaccaaaactatcggttggtttttgtttctaaaatacatataacattttcagtaacatccaataaataggatactgaatgttaagtttacgtgtatacatattgcaacttatttatcaaaatatcaattggacggttgaaatttcttttgttaagtccatgacaaaactaacgtcaactccaaatttttgatgcgacaaaacatagtagttagttaattagcactacaaacattagactaattttctagaactagggtcggatttagggtatttcatgccaagcttgaccgatcgactgaccgatccacttgctagtcaaaggtaatagattttctttttaatatagattacctatgaatgaatcttttaaatgccaaaataaggtacatcatcaaatcataattacttaaataatgcgtaaactgatcgtatgggttttgaatctctcaagctgttacggtggaactattggggcacttcgaagattaggacaatttctaagaagtgaaacgaaatacgttaagacctaatctctagaatatagttcccgtgtctttccaaagacatttgatgcctataaaatataaatcacatgactgatcggaagagtcgaaaaacacaaatagaactatggtgaaaattcaaatggttcaacagatttttattttctaagtattttaatcagattttgttcacaaagcaattctcaaatttgtgtacctcaacggctcagagactacagacacaaaggcgtgcctataagacgtcagcaatatcattcactgattatacataaaaaataatttttaattgaatgattagacactaatcacttttttaattccacatgattaattatttaattgaatcatctaataaagttttgtcgttggaggtcatttgaactccgtgtttcgtacgggttttcatattatttctgacctttgtactgcaaagacatgtttagcagctaagcatgtgattttgctagatatgcttttacgttaagaattatttattttattttaaagtatgtcttttcaggagaattattatttttttttttgttttttttcacatcatatccctctcaaattatttgtagtttatgtgtgatttgttttggtatatataatgtgtgaatttgtttttttagaaacactagagactccgtattgaaatgtttatttaaattatttgatagaactcttttttataaacacatgttaaaaagacggtcttgctctatatcagaaaataaattatcaaaacttactcctaaactatatccgctagaatttgtgtgtggagaaatatcgaaaatataaattcacatttgggataaacaaaacataaatttttaaaatgtcataatttaaaatttagtttaatgtttttgataaaacggtcaaatttcgttgatttggctacttgggataaatcattaaatttttgttaacaagtttaatttttattaaaaaataagaaagaatattgaaccaaaatcgaattgatgatcttttagactaatacaacaaactttaaaccattgaagcacgatagcattttcgtatgctagatataatactaggtagtaagagatgtgaaagaaaaaatgattgcaaagaacggataactttgactaatttcttactagatacgacaattttatcaaatatttcgaatcgtacaagtaataaaataaacaaaaattcaaatccaacgtttctgcgtgagatgcaacttggcatgtactttgttgatcaaatcatcgtaatcttttgtgtaacttatataacattttcgtcattttttttttaagttttccccctttttggtgactttctttgtcgattaaaaataaagtttgcacgatactttggaattagcttttaaattaattataataatgttgttcctatcttaagttgaaagtgcagggatgtgtacttgccaataatcttctagaaccagagtgaatcattgttattctcacagctttgattacaaacatttccccccaaaaataaaataatacaaaaaccatatatcaattgcaaatctatgggttcatatatggttttggtaaattcttttatctttggttgtgacccatatggagtatgaccttggctgtgaccttatttgtggattctaaatctaggacttggttatagtcgatagactcaatatggattgtaatttcataaataggatttatgcaaaaaatgtatatggggaattgtattagtaaaaaaaaaatcattttgcggcatatcttaccaaagttcgaattccggttattgcgttatcatgcataatagagattgaattatgcgacttgtccattccaataatgtcaaaaacaattgacaattttttataacactactgaaaataggcacatttaaacttttattatataaaattagaaataggcttacttagagggatttccataaacttttccaaaaactatttgtttctgcacacactctgaaagaaactcatctcttcccatctaaagatttcagactcatcacagtgctccttactcaagctattatctagaacatttagaaagagcagaattcaagaaccatatatgtttcacgttcgccggcttccatagcaagtgacatcataacaagatgtttcttggcctttcaaacttctatatttattacatcaggccattctccacatccttttggttttagcattatgatccaaccacaccatgacgatttcatgtgcaagtttcctgaaatcttcttataggaagagtttttttgccattataattcacatgtttttcttgtaattttttatctctctcttctttttattctaagtttcatagtgaaatattttgtaatgtcttacttattcaatagaagtagaataatatttattcacatacaactttcctaaacttgatagaatcctatctaggtaaacaaaattggtcaagaacttaaaccatctatttctttttattatgcgtcggataagacgtttgattttttttgaagtatatattcctcacatgtctttttctgaaacttttttcgtcactgctcttcttttttatataactacaatgtgttattgtgtcgaagaataaattcaaaacagacaacattatattatttctacgtttttgattaaaacataatcactttccctaaatcatccatcgaaagaatgattctcatcaagaaattacaagatggtagagttaatctcactttcagaataggtatttagtataaatccatcatagatatagataaccttatctcttatacaataaataactcttttgaccaaattaattcatatgctgcaatcttcaactctttatgtttaatttcgtatatacacctcaaacctaagagtatataaattggaatatcagaaaggatgcaaaattatcgtatttaattatatatcagaaaatatatacttattgtatactttttaaaaagaaatcgcatcacaaattgaatcaatatttgtgttctcctaatttttggaaagaaagaaaccaacttttaaccaggtcccgttggttcagtggatttgagtagtattggctaccgaggtttgagtaatttaacatagttttccgaacctcaggattaattttttgagtctaggaagcctttaaaatcacttgagttatcgattaaaaaagaaagatgagttattcaagataggattacgttatatatgttagataaaacataatatattatttacaaaacaacataaacaataatgttatccaaagaaccaactttttcatttaaaaattaggtaacatagtaaaattagacattccatattattattatatagtaaaggtacagtcattagactattcttaggataaatttcactttacatcaccggcctagcgttatgtagatttcggtgtttctaatacatgatctgagtctttttagtccaaagcaatcgtcatgataaagttgttcggacatgaaggaactagaaaattctactaactttggacaaaattcagttttaaatagttgataatataacccatagttataatcagttaattttctatttcctggtctatgtcgcctagaatgatatacgaattataaatcttatttgatagaaaagaaaatgttataagagacaaggcacgcaagaatgattgaagatggtagataatttaggaatccagaatacatgataaattattaccttacggcattcgaaaaagtgttcaaattatcaattcttattcttttttttcttataacttgtgattttgtgtgttttagtggattttctaatcattaggtaaaaagtgtggtcatttagtttaacaaaattgtaaatatcgaatttgatacaaaatgaaattaatgagtttaaaaataaaggtttgacaaaaaaaaaagagtttaaaaataaataaaaaaacttagagaatcaaaatgagagttttaattcttgtcatgttttgttttgatgcaagaaaaagtatccgaggatgctaaatagcttagaatgtcatgaatacgtgatgaaatcattacccaagcttgtatcatatgaaagtatttagggaaaattgtcaattcagaatttcttacaatgagtcaatagtaggaaagttttgtaacaattttataaaatttgggttaacatttgtaaggtaaaaaaaactacattgaaggaccactacatattcgactatattttattttgttttaataatcagaggtggtgttggaaacaacgtaaggttggattaagcaaatgtgaagcatccaaatcgttaattaattaatgacagcaaatcaagtttggctgttaaacactaaagaccaatccacctctagctatctattttactttgacgaaacccatttatgagttgtgaaataattactctttgattactttataaaaccttaacctaacaagtgaaactcttttaattacttttgcttagctcaagtctactggttgaaagaaaagatacattgtcttttctctattccaagttcgagtgtagtgaatggattgaaaactctagaagatatatgagtcatctaaaagttaagaaagtgagagatattgagtagaagtgcaaaaccttaggttaaggtcgagggtggtggggctatttcatcaacaatttgcgcacgaatgccaaaacaaagcttatcaattttttttggcatagtatatctaattagtgttgttaggcaaatacttacatcattcttttaaaatattctttcttctttttgttataaagaaataaataactattttttttcttcctttcccacaaatttattttttatgaccatctcatttttaaagataacttgatagattaggaactcacatttaaactagtttcaaaatctgtaaaagatctatctttactatttatattacaaaggcttatggtaatttccacactttataaatgggattttgtgcaaaagaaacccttcttagtcgcctatttttgcaactttaccctctatctccgaaaaatgcataataactctatgaagttaaaaataataaacaatatattggctattgtcaagtttttaaaatttaataagtaatatatcaaatcgagactctcgagacgaggagttcaactctttataattcctttttaaaccctttagaatatcatatatttcttctagagtgttactttctttctacaaaattattatctaatctttactctgtaatattacatttgaaaagttgcacattcatgtatgcatcatttcctaattttagaaaagatatatttcctagttaatgatatacggatcaattttccaaacaattatttgtgatatcttaaagaaactttaatttcagttgtccatgaaacatgtggtaatatagagagcattcctaggtaacaacatcaacttgtaatatatatattcctgaacaatgttacaagcatctaatgagcttcatagttcatcgtgcatgcgttgttgtagaggaatattaacagttggaactgtattgccatcaaactgttgtccgagcggcataacatcatctcctacaccatttgctgcttttccagctaattgagctttaatagcagcagtagccattcgttgcagtttcagaattccgatgcgttttgaaacaacgtaaacaacagcaaagacaaagagagaaaatccaactatgaggattatcctgttgatagtgccacagaaaacaagagttagttagtattattattcacctatagaatggtttggtaaaacagagtctattctcttttcaaatacctgtcaattacatcttgacgctgcattgttgaaagtagattacgggtcctcgacaacagagatctatgacctttgtattcactctcggcttttttaagtactccagtagattcatctacaaatgttaacatgtataataataataatatgtcagttttatgtcttaatctgttttcactaaggacttaattatctggcatattatgaatgacataccaaaagccacgagagtgtttgtacttcgttcaacctcctgtaacccaagtcaagagagtattttgatcacacattgtttataaattaagaagccgaatatgcaaacaagtgtaaatgcacaatcatttcataactagtttttttctgacctgaaccatcagctgccgtgaacgcctgagactttcagttatgctttcagcatcggatgtcacgccagcatttgctctataaacaaccacaaatgttagattcactggaaactaacactgaactctttttttagtgcatgatgcatttaccaataaacaaaagcataatacaacttaaacatgtccaagaggagtcccaattgaattgctggattttgtaggataaatatagcagagtttccaagatgtacatactgtcgtttacgtctgagaactgtagactctgttccaccaccgaggagaagctctctctacaatcattgaaagcactctttacacaaaaatttcaaaaggagctggctggtaaaaacagtggcagagagagagaatgcttaagccatgagactttacctcttcttgagctgctttcctcatgttatccttagcttgcaaattagcacttctcaagtttaccctcaaactgaccacaaacacagtgacaaaactcatattagtttcacccatttatctttccctgaactcatactctcactctcttgttgtaccagaattctaagtaactgagatcaaactcaatttcaaggaaatactttaaaacactcatatatcattgaaaaggatatataacattcctctcgttcttacaaactactacaacgatgactcaagtacccttatgccaaaaggttaaacgcacactatcacatcatttgtcctagagattgaattactcaactgcctaaagacaaccttaattgtacaaatgcaaggttcctatattctacttgtgaaatgttgtagattccacaagcagacagtaaaccaaatttccaagtacctgtgatattgattcttccacgtttcaaggagtgactgagtggattgaacctgatcatcagaaggaagctgaggtgcgagcagatccaaattgaattgaagggaattgagaagagagagaccatcctgagccaatccgttgagcctctgtaacgaaaacttctcttctcctccatctcccctccttgatttaccgtactgttgaatcgagataatatgtccaatcgtcttctcgtacgcttcttcccattctctcttcgtcttctccacttcaacaacaacctcatccatgtcgtaactgcaaatttcataaattataaaactgcagagtttagtaatcaaaagagatataaacaagctacggaatctcccaatgcaatctgctaatagctctctaaacatccaacacgtataaccatctaatggtttctgtatgaattatgatcgaatcactacaggaacggtatcagaagactcgattgatttgatgggatataagggtcacaaacttacattggttgatcgaaagagatgaagagcggagaaaatgagaatgaaattgcgaaactagggtataaggttgaagaagatctgggaaccagagagtgttggagatcaatattgatctgtagaagacacctcaaattttgatttctggggaagaagagaaataactaaaaacgacgtagttttagataaaccatgtctattctaaacccggttatcttgcaattgctatcattgtctggtccaatgtccggtttatcagttatttagttatatggtcatattcttattggttaaaatttctagcattacttattgatgaataaagaagtttactgctgagttttgttcaaagtacacgttaatcggatttatgaaacgacaattacacagactgacccaaaattggtaattgcggcatccgacttacccgaactgcatgccgttttgcaaaatctcaaaatgaaaaacggcatggtttcttagtaatctactctagttctagcaaactgacttgttcaattgcacatagagatgatcaactacaaacacagctaatgtttcaaactataagatgctgactacgtctgatgtactcaagcttgaggacgttccgaaattacggtgctttccatcggctaggttagccagagagtagccttgttcatggagttgattaatcaatagagtctctaactgacaagccatgctcttaccttgaaccataaggtatagaaaacttgacccttgcagtccttcctttgctcgatgcgcacgtattcgtccttcaagatcatcggtctgcagtttcgtaacagaaatcctcagaatcaataactttcgttttctgtctattctaagagaagttttataatattaatatggtaagtatgtacctgtccaatgtacaatctcttatcgggtctccgcatcacatacacgcatgaagaaccaactgtagatggaggtggaagctcacgagcaccaattgaaagacattctattgcttcaggctcaatcatctttttcccacagattttgacgatagcttttgctaagtccttctccaaacttctctcagagctgactggtttttggatctgctggtcattgttggatgaagttatgatttggtcgggtttgacaacttcagctgatgcgtcttttgcatagaccgagaggtaaagagcttcagctctttggataactgactcgggaacaccttccctcttagctgtttcaaacgcaagactctctctgcagactccatctgtcaatttccaagttggcttggtttgcccttcgacattttcggctcccattgctttatatgtgatgtttttcgctgtaagaggtaaactgaagattccatggagatgagtagatacaatacccaaacaaccacttgtgtcaagactctctaccacactaccagcgatacaggtgccttttgctgtctctgtccctcggcatatctcatctataagcactaggcttctcgaagtagcctggcttacaatagatcgtatttccgacatttctacctgatggcacagaaccagttgagaggatatcaaacattacaattaaagaaaaaaaaacagtttggtgaattctagatttgtatgcttatatatatacctggaaagaactttttccgtctacagggctgtcatatgatttcatgtgaagcatgatggaatcaaagtgaggaatacaagctgattcagctggaaccattaaaccggaaattccaagtagagcagctgcgcatattgatctgagcaaactcgatttaccaccaccgttaggtccagttagaagaaacagtgattgcatgtcaacggtattgtgaacagcggttccagaagatacatcaaaccaataaggtgacaggcctgtcagcttcattcgactggcaccatctaatggttttgcgccctatattaaaatatatcaaaataggtgcatgagatatataagaataagacagcccagcatgttttgtgatttcttagaaattgcacgcatctgtctacaaactcaacgactagcaagcacagttacctcatctaaactgaatccgacaagcgttggaaaaacccactttcgccttctcccttcactgtagttgtgcacagtacttagattaaaactacagaaatttcttaatgagaattgattaagcgcaaacttaccaagcatgggaaaataatgcttttgaaatgaccagaagcatagatgcaaagacaagaacatttatttttgtttgcaatttaacggataactcgcgcaacagttccaacacccgagcttttgcattctcactagcttcatggtatctgcaacaaaagcataacatgatatatcaatacatacccagtattattgaataaatttcttttggatggttcaataaaacgaggaaacaacctgactaaagcaatttccacctttggggtcgtaaaccattcttctccaaccttttttcctttcgagtctaaggcaggtttcagctgttttatttggtcttcccctgcagtaccagcccagatagatggcgtaaaccgtttccccttgaaccaaacagactcatgctctcttgcatatgcgatttcgcctttcgggccaccaagtgaagcagtggtggccttaattcttgatataatagggtgaaaatcctcagctacctacacatggatgttttgactcttacaaatacgacaagatcaccagcaccattatatttaagcaaaaaagtactatatgagcatctgttatcgttcaaagaaataagctgcaattgttgaatacttactgctaaagataaagcctcagctgatttttctacttgagtgatttcttcctctatatgaattcccttaacgcgacctcgccatgaagactccatatcataaaagaattcgttcgggacattgtcacatttacttacattctgatgactttcattctcatctaaagagatcatttcaccaattgtatcagacgcccaatgacattcgttgacctggcaagaaaccgaaaactacgatttatactcatactcataagctagttggattataaaaaatggcattcaaattcaacgcttattttctgaactcgtgaagggataggaacttatcatcaaggaaaatagataagaaaacaaagcagggagacatacactcagagtcatgtttaaatgcataaacttacaaaagtgtcaaagtcaattttcaaaccagtagccacccaggtaggatccatcaataatttcaggatttccacaagctcagcatgtctatgcatatgtaatacatcatcaagcacattttttattcgacagaactcaatgtagttggcttcccgttgctcaagaagcttcacaagctgcaaaaagatgcaaataatcacactgtaagagatgagagttccataagtggaacgacaaaagttagcatctttcagaagaattctatgtaggaagtgacttaaaccttagcagaagagacgcaggtaaactctggaattgaacatgttactgtgctcatgagcttgcacgtttctgcaaataacaatagtttgacagggacaatgagaagccttaatcataagaaaccctaaaaggaataaactaaggaaactctcttcaaatcaccttgaattttcagagcaatatcgtaagcaggagggttcagaagaagatccctaacatacctgaaaggataaaggcattggaagaagtagaggagaaaaacaataagaacgtaaataagatctttcacccaaacattacttacaaagaaggcagaccactgcacgtagatggaagtaacaccttcaacaaacaaggtattccttcagtaggtaaggcacctgcagaaccacataatatatattatcatgctttgtcattcataaactcctaaacctcctagtgaagcactattcagcatgaagaactatgccaaccaatttgtgtagccgttccaagatgcaacggacgtggccgattttttgaaggtacattgacatttctaaaggaaacttcatcatcaagaccataaacatctttgacctgagggggaacaatagaaagtagttgtttttcacaaaactgaaatgtcacaattgcatatttagtcagcagccaaagtccgtcgttctgacccttgataagagctcggaaagagtatctccttcaaaccattcaaaattcctgctactgcattctccccagagtagacccccttccccaaactctccccagcggcacgtccctgcataataaaaaattaacaagaattgtatcagaaaagaccacaaagctcaaaagtcaattaagttgacacaaaacatgaatcgtatttctcttgtaactgtctccagactctctaaagtatttgctagatcatatgctaccacctgagaatacttatgttcatgtgaggataaaatggcacccgtctacattctattattggcactatcttcaacagttcagagatgctaagacaatgtagaattgaagatagtattcaaaacctgatgcattgtgcctcaacgatgcatgtaagaaaagatgatgacagcgacgagtgcggagcttggtaactaaggcttcttctgttagaccatcatctagcgaatatgctttcatagtctcgaaaatagatatcatacaatacccccttgctgaacgagatatccctgtacttacagaaaaaatcaagtcatgtaatacttatatataaatttttaataggttgtagcgcaaaataatatatcactgactaacaaaataccataagaaatacttaccaacaacaggcataggatcaggaaagtcaagatcatggtcaacaccgacaagcccatatacataaggacttcctggatgtgcatgcctatatacagtacatgtcagacagacgaatgcaagaaaaaaaaagtttcagtgtattttcctagctaataagataatgaatattgcagcttgtttaatcagaaaattccaggttctatcatattacataccctgaaataaatcgacctttacgggagcgtgctggtgttggcccctgaacttcctccacaatacactacagtgaagaaaaaaacatgcagtaatttttatttaacatgcttgagggacatggtaaatggtaaggaattatttttctcaactcaccactgaataaccattgcgtgtcaggtcatccaaagtctgtcgaagattctgtagaaagaaaacaagaatttgtggcaaagaaaaaattaattgtagacaacgaaagcctgaagggaagagtaatgatgattaattgcagcagaacatatgcttgctatccaacaaaaagcaccacaacttgattccgtgtacgcaatggccactaaagtagctgtttatgtaacttgcccaagttctgcctcaatgattacaactctcagagactaaatagaatcaaaaaagattataactttactacaagcaaagattaagaagctataatggctgtgtatatttatttgcccccttaagaagatgtggctctcctcttgtttaacagtaattttctcaacacaataccagagacgaattagtctattttatcataggaacaaattataaacttgaatcacataaatgctcataatgtgagtaagcttgaaattcaaaaaggattgtgtaccataattgggcagccagcctttggaatactatctgatcgaagaccaccaaaaggattgagaccagcatattcaacaagtatacaagcatctattccaatagcctcataaaattctcctacctggagaaacaaatttcagaacagtaacagtcttcataagataaagacttcaaatgacattattctgctaatttttgttctgaagtaacaaagatcacaaaactcaatggaattagaaaaacaacaaacatactctgcagagcaaaacttcgcgtggaaaccttgacttaaactgcaacatctcccagttgaggtttccatcttttaaactgaaatgaagaaaattccaaatattgcatacataaaggccacaaagttaccgtaacaaactggactaaagtagtggactaggaataaaactaatggattgtttccaactaaagtacttagagccttcacaactaaagtagcttaagtcatactaagatacactagctacttttcatttgataaccaagctaaaaatgattaaaggctgacatctagaaggtgagatgaaatcttctaatagtctcagaagagaactgaccttccattcctcaagctagggtccaaaccaagtaaattggtgtacataagcctttcaataagctgaagagtagatggtttcttacatgtctgcaatctctgagtagacacagaatataaaaaatcagtatataacgaaacaatatcaagacttgaaatttcttaaccaaaaaacaaaacaagctttacctccttccaccaaaccaaatgagagagatctttgtcagtgagaacatcacttgacgtcttcactttcttagaagccgttgtgattcttttcaaagactttccatctctgagacaagatatcccctcagagtaccttctattactgtcaacacataacaaaatacaaaaaaagtttcataacaagattcttatcaagaactagaccaaacaccaaaatcaaagaaattgagttcaatattcaaaagtacaggaattcataaaatacaaaaaatctgaacttgctaacatattagagaagattatacaaggagatgagaggattacagtagaattggggaggagggtttgagggaagagtaagtgcgatatgaggagcggaagaagaaccgccattttgggaatgaaacgacggcgtttctggtagcaatccaatgcatctctctcacactctgaagaagaagaagaagaagaagtagagttttcacctttcaccctttttctagggcttcttttcccgccagaagacgacgactatgcaattcacaatctcacagtcctctaatggaaaacatgttttctcttctcttcttgtcttcttagtttaaaggtaattaggattttagccttttaaaaagagtaatttctcatttgctccctataatgtttcaaattacattcttgttcattaatttaccaaaattaatcatcggctagttttgtatctttttggaatgtctaacaaagaacccatctctatgaacacatctctaatctctcagttttgtttgtaaagattgtaatttctctgcttttcaaagaggatcaaaagttttgtaattagaaatcttaaaatgactatcagttgtttctgaattctatacaaacttaatactctaatgagtattaataataagaaaacaaaaccactaaatgtctagactcagtgcagtgtgtgagtgtgtgtgtttgtttggcacaacaaatacgaaacatctagaattacataagaatttgtggtgagtcaatggatttatcagataggcatgcacaaagtgtttcaccaatggctattttgcatgttgtgtctgcttctttaactgaactaagtacaaatcacaaatatccaatcacatttcatcttctcccttaacaaagactccataaagaaaagagaagaaaagactcataaacagagaaaaaaatggcaggagagagatcaaagttaacaacaaaccacttttataatcatcagattattctttgttactttcttatcatctcacaagtttccattgcctcaagcaacacgagtaatgtaggagttaactggggaataatggcgagtcaccagcttccaccagaaaaggttgtgaagatgctaatggacaatagtttcactaagctgaaactatttgaagccgaccaaaacatcttagacgctctaattggctcagacattgaagtcatgataggaataccaaaccggtttcttaaagaaatggctcaagatacatctgtagcagcttcatgggttgaagaaaacgttactgcttattcttacaacggtggagtcaacatcaagtacatagctgttggaaacgagcctttccttcagacatataatggaacttacgttgagttcacattaccagctcttatcaacatccaacgagcactagaggaagctgatctgaaaaatgtgaaagtcactgttcctttcaacgcagacatctatttttcccctgaagcgaaccctgttccatcagctggagactttagacctgagctaagggatgcaacgattgagataatcaatttcttgtattcacatgattcgcctttcacggttaacatatacccttttcttagtctctatggaaatgcttactttcctttggattttgccttctttgatgggactaacaagtctttgagagatggaaatttggtctacaccaatgtgtttgatgcgaatctcgacactttgatttgtgctatggagagatatagcttcttggggatgaagatcattgtaggagaggtcgggtggcctacggatggagacaagaacgctaatgtaaagagtgcaaagagattcaatcagggaatggtaaagcatgctatgtctggaaatggaacgcctgcgaggaaaggagtgattatggatgtttaccttttcagccttgttgatgaggacgccaagagtatagcaccggggacttttgagaggcactgggggatctttgagtttgatgggagaccgaaatatgagcttgacttgtcaggtaaaggaaatgacaagcctttggttcctgtggaagatgtgaagtatctgcccaaaacttggtgtattcttgaccctaatgcatataacctcgatgacttgcctgataatatcgactatgcttgtagtttgtctgattgcacagcactcgggtatggatcctcttgtaaccatctcactgctacaggaaatgtttcatatgcctttaatatgtattatcagatgcacgatcagaaaacatgggactgcgatttcttggggttgggtttgatcacagatgaagatccatctgatgaactttgcgagttccctgtaatgattgatacaggagattcaacgaggttgcagcctggatcttcaagagtgttgaccagagtcgcagccgcggttttagttatgttggtccttcccatcttgtagtaagtacctgttgtttgttatgtcttgtaaatctctcaagttttacttcctgcaaaagaatctttcaaactaaaggttgttcccttcgattagcatagccttggaaaatacacaacttcattaactagaagaaattgaatcacagaattacatattactagagtgatctgttccaaagctggaagttaagtaatataaacacctagagtctcagagtgggattaacagagagaaatgatatttcaatcagaagaagctggtattcatgtcagatttctttgggtgataaagtatcttctcaaatatgtgtttcagcttctcgtgccgaaccatctcgtcgaggattctatctttggaaacaacttcgttgttaatcggcttgtccttctcattggtagaagagaagaccagttcggatatgcgatgtttctcactttgcttacagtacttgttccactcagatgtgtctttgaccatgagatggtaaatgaacacagcacgcttttgaccaattctgaaagctcgacttatggcttggctttcaacagaagggttccaaacaacatcaagaatcacaactcttgaagcgccaacaagactgataccctcagaacatgcttttgttgatgccagtagaactttagatccactgtctggtttgttgaagttgtcgatcatatgctgcctgtctctttgttcaactttaccatgcatcaagagaatttgttccccttcagtccagtcacattctgcaatgagctgctccatgatcagcttcagagtgtcaatatattggctatacaccaacactttctctttcacggttccactgatgcggatgaagtcgataaggaactttgttttcacaccttcctcgtatttaagtctaagcctcttgagagttcctaatgttgctggcccaataaccaagtcttccttcttggtcggattgcagcacaaatacagagatgggtgtactgaaacagctgaaagcttgtgctcaaattcaaatgtgttttgagaagtgtcaattctgtcaagaattttcttctgttgaaaaggcgggttcaacacgacaacacagtctcttagacctggaaggctttcttgaaggatggttccttcatggacatgcacaaaatgagcaatcatagccttaagatccacaattctgttttcctcattcacccttccgtgttctccttcttggctacacttgctaagttcatgtatcctcgaagaaatcgtatctttatcagctggtcttgccaggcataagacgttagaaagttccttgaaattattctggaacagtgtccctgaaagaaagatacgcttttctgttctgacttcagtaagcactttccaaatgagactactctggtttctcggggtgtggccctcgtcaaggaccagcaaccccggcaattctaccaacatcctcctaaacacttgcattccttcagtgttcttatttgccgcgagcttctcatacagagggtagctaattccaagaatgcttttctgcttccaccacgaaactagcttaaccatacggatggaattatgatgcctatttccctctaaacgtgagacagcttcagcatcctcataccctgataactgcagactattcatgttataaaatggaatgtttacgttccatttcctgacttcgtcttcccaagtacgcattagagttgcaggagctatgaccataggatggctatttggaaaccgttttaagtatgactgaagaaaaacgacggtcaaacgtgtcttaccagttccagccttatgcgaaataatgcatccgccacttcccttaacccctacgctgttcaactcattaatttttgttgtcccagctaaattcttccaaataaattcaaacccttcttgttggtgtgggtacaaagtatctttgatgccagggacgtattgccagacagttccttctatattatccaaaggggcgacaaaactgctgggatctgaagcatcaaactcaagcctgttaggtaaagggtcaccctttctatcactacatttcttattgtcattaacactaggacgatacttgtcctgcacaatgcaggaaaatgaacatgttaagaactcagataagaaaatcaacaaatagtacaactcaatctaggatttcagagcgctataaaaatgttaccatggctggtgagatgtctttgatctcgacagccacatacgcacagtggacgcatttcaaaccgatttcatcatccaggacaaagtcgtgtgtccctttgctacatagcatatctccattctaatacatcagagagcaagtaatttaatttcatggtgaatcaatgatacaaaaaaaaaaggtggagatacgaaccaaaaagcacgcaacaagattgtcaggcatagcctgggcaatatagaagaattagtagtcttactttatcaggggtagatgagtgcattccttccaatgttagagcaacgttcatatcttcccacaagctgtctaattctttctcctcttctgttttctcgataagcacaggctcctcgcagccaaatctcaagtttaaaggaggtgaatcttctgtcgagtagtttattgtctcttctccatcacagagtttttcgccaccataaaaactctcaccattcaagtggttcttctccctcaccctatggaaactcctccgttcccttggtttcccatgttcatttaccttctcacttgagctctccctaactacaggatcttctctagaatcaacctctgctattttgtcccaggagaaaatatcttccttgaaaacgtctttgctctccaacatagatttagctagcaggttaattacatcgaagttatgcttcctacgaaaagtcctcgacttcttatgatggtatactttttcacttgggttcttttcgcatgtagcatcgtctctggtaccaccctcttcatcctcagaacaaacgaaatcactatcagagctctctacatcagaagaatctgaactttcccccacgtaatcagatgagtcagaatcattagcatcttcatccatatcactctcaccagaatctctatcttcgccactaacttcctctcttgaatcagtccctaattcctctaaagggtcttcctcatcatcagaactagaacttagacttacaacctcatcagacacataagtcttctcctcacccctcaaattagcatcatccagaagaatatcacaaacctcagaaccagcattgacatcctcgacatgctgattctctcccgcgatagttccaaggaaaacaacatcatcatcatcatccggagataaagggttctctttaccactcactttagcatcatcctcatcaaaatcacaaaccctaggagaaatcaccgaagcagaaccaacattgtcatcatcctctacatgatcattctctctctgaacagttccaacgaaaacaacatcatcatcatcatctatagggttaaaattagaagctttctcttcacatcccaaattacgatcatcagcatcaaaatcacaaaccctatcaccaaaatcaaacgatttcgattgtaaattcccagaagtagaaccaacattctcatcatctcgcttaccttcaggatattcagttctaacaaatacaacatcgtcgtcatcatccttcctccgccgtcgtttcttcttcctcggagaaggtgaacaagcatcacgcatgttaactctcctcttctcagtcctcgaatttacacaacccagagactgatcttcttcttctccagaaatcccctttgatttattgagaatcgagttcaagtaagattcagtcctcgaacgagttcttctagcaacacaactagtcatatccatggctgaagagagaaatcgaaaaccctagaatagtggatttgacgaagaaggaagaaagctcatgtcctctctcctctgttgtctctgcaaatttttttttttttgcctttggcggtaacgattcaactttctcgaaacattcaataaaaaaggtttttttttgtttgtttctttattagggtaaattgtaatatttgaaaagatgaggggctaagtgaaaattaatagataagtttgcttcatttctattggtgggaattgattgacatgagagtttcaaagttaaaggtggatgttactggttctgtcagtagatagaggtatttaataggggtcaaaatattagtggaaaaatgaaaagcttttagtatttcaaatatattttacaaattggttactaattttttttgttgtaatggtgctggtaaggacaaaaattctattatttgcttaggattctctcctttatgttttgatgtgagtatgtgactaatatttctgcaaacgatttagtgttgggttattagagaaagatatcttctggagaacgaaaatctggaggtgagatcattgctgatttgaattgtaaattttttatgaaaattttgtggtaatcaaatctaatggcgaaaattcatagacagagtcactcacagagatatgcaatttatacaacttttacgattatatattttttgcggtttaatctatcaaaacatgtattataacttaaatagaagattattattatttgtttgtttcaaaaataaaaagagtttgcggaataattagtagaacatgttagagttaatataaatatgatagagaatgagttaaatagaatgttaactttgacacttctaaatttataattacagtaataaattatacataccagaggctatatataaaaatactaaaaaaataaattatatcactttaatacttatttaaaatgtgtaacaatctaaaaaaatggtctggtagtatattacgaattaaattttattagttatataaaaaaaaatatgatgaagaggctcaattcctcaaagcaatttatgtgagagttcaattattattagatttctaaaattatatttttgagattggatttctaaagttctatttttgagtttgaactttcaacgagttataaaattggaaatacaaaatcttatggaatcgaattatctaaccgggtttaggttccaccgatcagactaatcggatcagctcaagttctaaaatatgtaaaagctcgtaacgagaactttcaaatttggggcaaaaaaacatgaactttctaaatgtcatttaaatctcaaagtttggtttgactttataataaaatggttaagttttgttgaccgagtcattttaggcatgtcgttaaatttttgttaattaaaagatgaccgagttaacttttcataatgtttgggtttagaacgataccttatatgtatttagaaagatacctcataagctttctgataccctacgttgtgatggtttttctctttaatgacatcatatatgtatataagatttatttatttcccaaacgaaaaattaactctgtcgtctttttattaacggaaatttatcgatatgcctataatgattcaatcaacgaaatttactattttattataaagtcaaaccaaaatttaagattttattaacattttgaaaattcatgtttttatgtcccaaatttgaaagtttgcaaaaaaattttgacatttttcccgatctaaatttaccatttaaacaataaaaagtaaaaggtcaaaatgtaacattttagaaacacaaaggacaaaatgataaatatcatgccaacgcgttgatcggtttttagctcttttcttccgatttcgttttttctttcttcatctctaagattaagcacaccttggcatccattttcgtttgaatctcaacgttttttgcttgattttagcttctgagagagtccagatccaaggaattggaggaagaataatgtcggcaaggcatgggcaatcgtcgtatcgagatcggagtgacgaattcttcaaaattgtggagactctaaggagatcgattgctccggctccggcggcgaataatgtgccgtatggtaataatcggaacgatggtgcgagaagagaggatcttatcaacaaatccgagttcaacaagagagcttctcatattggtttagctattaatcaaacgtcgcagaagctatcgaagcttgcgaaacgtaagttgttttagtgtttagctcatttgatttggtgagatcttgtgaatctggattagttaacctgtaatggctttagcttaggaattagaatggtgcttagatcgagaaccgatttatttagtggtgtgaatttctcaagtgcttatcttagtactcgagaatgtgtaattggtatggaagatgctgtattttgtgcataattgttaactttgtcgtttttgttgcaaatcagttgcaaagaggacatcagtgtttgatgatcctactcaggagatacaagagctgacggtagtcatcaagcaagagatctctgctctaaattctgctctggtcgaccttcaattgttccgcagttctcagaatgatgaagggaataactctagagacagggacaaaagtactcactcagcaactgttgttgacgatctaaagtatcgtttgatggatactaccaaagagtttaaggatgttcttaccatgagaaccgaggttttttctttgttccgtcgatttcttttcgataaagcttgagtatgcagtcgtgactgagctatgatttacttgtctggtgttgcagaatatgaaggtccatgaaagtagaaggcaactcttctcttcaaatgcttcaaaagaatcaacaaacccattcgttcgccagcgtcctttggctgctaaggctgctgctagtgaatctgttcctcttccatgggcaaatgggtcttcttcatcttcatctcagttagtcccatggtaaagtactattaaccgaataaccacagttgcaaatacagcatacttcttgaaatctggctaagaatttggtctaatgggagtccagaaagttgtcatgggtttactagaatgttttctgtaccatgtcagtcgttgctgatttaagatctggcttgtcaagaactcaagacgtttcataatctctgcttttacaggaagccgggagagggagaatcttcaccattgttgcagcagagtcaacaacaacagcaacagcaacagcaacaaatggtcccattgcaagacacatatatgcagggtcgagcagaagctctacacaccgttgaatcaacaatccatgagctaagcagtatcttcacacaactagcaaccatggtttctcaacaaggggaaattgcaatcaggttcgtcttcctcctgaggaaaaacatcaatatgtgttataatgtccaatgattcatagaacatttgtggaaaggaagtataatataatgttgctggtttatacaggatcgatcaaaacatggaagatacattagcaaatgtggaaggcgcacagagccaactggccaggtatctcaacagtatatcatcaaaccgatggctaatgatgaagattttcttcgtactcattgcatttctcatgattttcctcttcttcgtggcataaacattgagactaaaaattgattttaaagagtttcaacatctagacatggttcttttgtcctatcgtatcttattgacatgtaaagacttaagagaaaaaagggatcttatgctctgttcagattaatggagagatatataatgtcactttcatgtaaaattgctttcaagttttccaaacaaatttttgtgattctgcttctcttttcattaccactgccaaccattaggaattgtttcttattccatttgtacttcatttattatgtaaagacaaaaaaaataatttcattaccgtgtttatatatttatttgtttgtaagttaatcaatctacgacttgaaaatttgataaatatatgggatatgttcaaaagctgcaacacaataatcaccaattcatgatctaagcaatatctctgttgtttggttcatacgtttagctcttcaaattcaggacttaatggcataaagagcgcgtccacttcggtttcatctacttcgtctaagctcgctggtttccatttcggattctgcaacaaaaaacacactcatgaataagcttatagtaggcactacagagacgaaagagtgtgaaatgcagtgagtaggaagataaaaatgacagagacattcttgattttataaactatgttcatctttgacgtgtgatatatatatactcagcttcatgtttttgacccaactcaagaaattagaaagcacacaaaaaccaattgacagaatgaactaaatcattaaacattgagtggcaatgaacaaaaacctggtccttgtcagtcaagactgctctaacaccttcagtgaaatcactgcgcaatgcagatcttagagcaatgcggtactcagttatcatcacaccgttaagcttcaaaatggaaagaatagtaagcattacgaaggaaaaactcgtaatttatcggatacaaagcgtttgaactcactgttgccatggcgttgttggttttacccttggcacaagcaactttggagaagtacttgtgtgttaggtaaagagaaaaaggtgcccctttttcaatgccttggactgcttcattagcccattctgcaactgtacgcaaacattgtgtccaaatttctcagacacaagacaacgatttcaatttcattaatgtaaacagcaactaacagtagtctttcttagaaatcaagattggtaacatacaagcacaaagggtttggtatttcaatataaaaggcagaaaagaagtttctttgtcgaaaccttaagagactaatgcatatcatacctgaagcctcactgctctgctggaactttttcagctcttctattgtttccttaacagacttacttacactaaaagctgattctatctgaggcaaaagcatttggaggtgggactcagtttcaggatcactgctgtaatttgataaagttgcttggatgtgttgttggggatccttggaactgtattaccaacacaaattctttcttagagactaaacaaagtaatactaaaagctcctatatttgaaatatacacatgaaatatatcgaacaaagtattatacgagggcctacaggtcagctgacaaaatggcttctctgagtgaacctagctttccagatggtacataatgtgtcccaagacccacaaacagtgcatctgaaggtgtggaaatccttctcccagtcatacctaggtaagcacctgcagaatttgaaggttgaaaagttgctggtggataatgatactacagcaaattgttatgcaaattgttgagggtctcagctatatgaaacataccaacagatccttcgccagggctatgagctgcaatatatgaaaatccaacatctggaaacaagccgattccattttctggcattgcgaggaccgtcctctggaaatggagaacaaaacagtgacagaggtaagaaatgcatgataaatgccatccacacaacagtacataacaagggtttactatgtgcacctctgttatcactcggtagcgtccatgtccagaaagaccaagaccaaatcccattgttatgccatccattaaagagatatatggttttctgtatccagcaatcttgcatatcaggctatactcagctgtaaatacctaaaaggaagcaaagttatgaaattaggacatcataaacaagaaacatacattttgaagggtaatcaaatgaactaaagcaattgatggtgaataaaaattgtaagacactgaaggtgactacttcaaagaagggcagatccaagcaaaaaaagagaaagcatggagtaaatctctagaatcctagactaaagagtgttaccacacagattattttgatcaacagaaataaagaatagaccacctaacatcatcaaactagaagtttcctactacttcaagtacattcagtagaagtagtatttctctatttctgtttcagtaatcatcatatcctgacttatgggggaaacaattgcacaaccagacctctatcatatctgacaattgatttttggatgtaatctgcttcacatcaccgcctgcagataaactaagaccatggttataaaaatttctatccagtgtgaatttgataaagaaaactcctttgatgcaagaaagggaaacccaaaaaacctcacatagaacaaaacacagagtacaaaaataccaaagagttgcaaaatgacattttgtatgtttaaagaaagagataagccttgatacagccaataacacacaattcatataaaaaccttttgcacaagagacgtatttttgtccattaggatctctgcaacaactcctttaatatccattcctgcaccattcataaatggtcactctaaaaccttcccatggcattaaccaagaccaaatgttttctataagataagataagatcagatgcaacatactttcaacaaatttatcctacaatcttagaaaccagaatccactatataagcacactcaagaaaagtttcttttacctgcacaaaaggcccgagatgtgcttccttcgacgacaacacatttgactccaggatcatattcccattcatcaagcaagcttttgtacttcaaatccatttctgtaaaaaaaaaatcttcaataattgaacttgggttgtgaaaaagcttaattctttatgaaaacggacctagattcatggcgttgagagctttaggtcgatcaagagtaatgagagcaacgccatttggataaacatttcctttcacgaactcgtcgcttccactggccattactgagaatttccgacgatgagatacagagagctttgagaagggaataagaaaagatggagaggttctagagaagaagatgcgagcagaagccagagataaactcttgatcatcactgtgattcctaattccacgtttgtcggagaaaccaaacatctgtgactttgttttcttccaaccaccaaaagtactctgtgtcagtttaagggtccactaatctggagatatgtcgttcggctcaatacaaaaagcccaatagtgaccaataacagagttttgtttaagggtaaattgcaggccacatccgttgaccatgttttatttccatggattgtcaaagtcaaatgtggttttcaccatttgtcttttcgcctgcaaaataacgcttctgcccttgcgcttctctcttagcaactgttgaaatgtacggtttagtttagcaggtttagttacgtacactgttccggttcagttgaagattttttgttcacgttgacgcgattcggttccgttacaagtgagattattccgaagtgacatcaacatttaatgcccccaaaaagtttagatgtgactattgtcacaattaatgcaaaatcatcattacatcaaataatattaaaaaaattattaataataataaaaaaaaaatttgttgagataaagaagatgaacccaggaacaaagaattattgcgaacgtacattgctgctgaagaagtgttgatcttgtttttgtaccaagtttggagcattgctcgtgtaagtaatgaagttcttttaagaagttttttttatgtcatattaggatcttgattgtgttcgaactccatttttttatcagaatatttgcaaaatctatatttgtttgtttaaaatagtcaaatgataattggtatttaggtttataaatgtgtagtatcgacattccatttgttttttatatgtatagtatttttcagttgcatttaatgtgtgtctcctgaatttatgatgcaggtgggttacggatattcccgtatttaccatggtcggtgagtggaaatgcaaagaaggagaatggaaatttgaacctgaggaaggcatctttggtcgttgtgtacgcgttcaggaaaccatgacgtacacggactttgtccgtacactccgagaggctttcagtttgaagtccaccgagatcaatcccataattagctactggatgccgggtgaaatgtcagtgctgatagatactaaacgccctcctgtatacattgacagtcagatgggtctcgagacgttttttttggttcgtggtgggtatctttctctcaacttgttcgtctctttcaataatacagtgaaggcgttcgggaatactgtctctaggtctcatggtgaagtttctgaaagtgaccgtgtgtttgacagtgctacaaatgttgatgaaaataatgaggatgcagaggagcaagccgatgttgaagacgatacaacagaagaggatgaattggaggagggcaacgaagatgatgatgggtgtgatgattatcttggtgtggatggatctattgggggaagtaccctgtatcctaccacagacggtagtgaaggtattggcgaagaatacgattacaacaagaggaacgatgtaatagtagaagaatatgtacaaggaaatcttgaggaagacgtcagtctcactcaacagactgctcaacatccagggaaattcagtcgtttattggaaacgtgtacgtacacgggtgttagtgtacctggcggagatacaggggaattcagtcgtctcttggaaacgtgtacgtacactggtcttagtgtacctggcgaagaaacaatctctccattgggttacggtacacgctgcgaagggcaaagggattccaatcttatttgtatagacgaagtcaccggggatgaagtgatcgaaggtattggagcttcctcaactgggacacaaaatgataatggggtagttaccgcagctgaggcctaccaagtgtataatgaatttcctaagttacatggtggcgagttggcgaatctcggtaacgatgctgctcctgtgttcgatgacttactcaattttcgagctgatgagacacaggttgaaatcagcaccacaggcgaaactttgtttgttggaagtgtttttaagaatagaaaagtcctgcaacaaacaatgtctcttcaagcgataaagcaatgcttttgcttcaagcaacctaagtcatgtcctaaaacattgaagatggtgtgcgttgatgagacttgtcaatggcaattgactgctcgcgtcgtgaaggattcagaaagtttcaaaatcacttcgtatgctacgacacatacgtgtaacatcgactctcggaagaactacaataaacatgctaattataagctcctcggagaagttgtgaggagcagatacagttctacgcagggtgagccgcgagctgtcgacttaccacagctgctcctaaatgatctgaatgtgcgtattttgtattctaccgcttggagagcaaaagaggttgcagtggaaaatgtacggggggatgagatagcaaattacaggtttttgccgacctatctgtatcttctccaattagctaatccgggtacaataacacacctacactatacaccagaagatgatggtaagcagcgcttcaagtatgtctttgtctctcttggcgcttctatcaaaggtctgatatatatgaggaaggtagttgtggtagatggaacgcagctagtcggaccttacaaaggatgtctccttattgcatgtgcccaagatgggaacttccaaatattcccaatagcttttggtgttgttgatggtgagaccgatgcttcttgggcatggttttttgaaaagttagctgagattgttccagacagtgacgatttaatgattgtctcggacagacattcatctatatacaaaggcctaagtgttgtttacccgagagcgcatcatggagcatgtgctgttcaccttgagcgaaacctctccacctattatggtaagtttggtgtgtctgctttatttttcagcgctgccaaagcttatagggtcagggatttcgagaaatattttgaactgttaagggaaaagagtgctaaatgcgcaaaatacctagaggacataggatttgagcattggacaagagctcactgtagaggagaacgctacaatatcatgtctagcaacaactctgagtccatgaatcatgtgttaacaaaagtaaaaacttatccgattgtttacatgatcgagttcattcgggatgtcctaatgcgatggttcgcatcgaggaggaagaaagttgctaggtgcaaatcttctgtgacacctgaggttgatgagagatttctacaagagttgcctgcgtcaggtaaatacgctgtgaagatgtctggaccgtggagctatcaagtgacaagtaaatccggggagcattttcatgtcgttctggaccagtgtacgtgtacgtgtcttaggtacaccaagttatgaatcccatgtgaacacgctctagcagctgcaatcgagcatggaattgatccgaagtcttgtgtcggatggtggtatggccttcaaacgttttctgattctttccaagaaccaattctacctatcgcggatccgaaagatgttgtcatcccacaacatatagtggatttgatactcattcctccatacagtagacgcccaccaggaagaccactgtccaaaaggatcccatctagaggtgaaaaccgggtatgtactaacatcgttcattcatctacttgtattttgagtattaccatgcggagatttgtaacttcctgtctttgtcacttcagaaaagcaggtcataacaagaagacgtgcaaaaatccaatctagttccggtgacaagtttctgagccggccaatatagtgtacgcttctgtgttttctcggtgtacgtaagtttatatataatccacctgtaactatgtacgttgtgtacgtacgtttaaactctacgttcgtataactatgaacgtttttgtctgtgtacgtgttaaagttcatgtaataagggtacacagaggtggttgctcaatattttgagtaatttgaaataagttttttgaaaatctaacttattccccctcttaaatgaacggagttggagacatggttgggtatattgtggttgtgttggtatttaatggaaaggacgtaactgttgctgtttattgtcaaatcaacgttatgattaccctatatgacggttaccaggctattaccaaaaatttgaatcaagtgtagttgtagccgcattaataactgtagccgttccagatttcctctatttaagtctcctcccttccttaattttatccacaatcttcattctaaacccatttcttgtgccggcagttatatcattcacaaaaaaaaatttagtctttcgaatcgcttgttcttgctatccgtgagatggaggggtttcatacggtctcagaattgtgtcctctgatcactgggtggcgaatttatgtctttgtcgtgagggtcttcaagaaggttatatcgcccaacgtcttcgaactcggccttatcctggcagattacgaggttatttccgttaaatcattatgtgtttttctcgttcattcgcttaacttatatattaaactctctactatcatgttctgtatgtaaagaataaccgtattgaggccaccgttgatcgtcgccttgctccgttttacgaagaccgattcgttgaaaatgagtggaagacgatcacctctttcttggtctgtaagtcaactgacttagtcagagcaacaaagcatgaatacggcatactgtttatggatcagatcgtcgtcgttcatgccccaccgagatcaccaccagttccagatttcgatttcactcccttcgactacattttggataaatctgcttacaagaacgttttagtcggtgaggaggaataactctgacgcttcatttattacatttttgaaaatgtttgtttattatctttgtttctccatcatctagatgtcattggagcgttggttgatgttggcgcattgactacagactattacggtctcaaactgagtttcaagttaaaagatagatagtaagtgttcttttcatttgttttatttacaatgtcactctaaacgctaacagtccttatttctaacgtagcaatgaagtcttggagtgcgaagcccgcaatcaacatgcggagtacttggatggttacttttagagtttgggtaaaggaaattttgttgtcgcactcagcttttggcgtctaaccgaactgtctaacccgaagcttgagagccatggtgctatttcaaaggtggttgccaatccggacagagcagaagttgcggatatctcgatggtggttttttagctagagggaatgaaccattattctccgttttaatataatataagactacagttcaattaagttttctttcccagttgttgttgttgtttccataagaaccaaactacatttcagtcgttgttgtttcttgtttccgtttaaattttaatctaatttattgttttatgtacgtgccttgttttattgttcaaatcctctcgcaatgtacgtatagtttctggtgtacgctatcgtgtacgtatattagttttggtgccttcaacatcaaaaactgttagatttatacttatgttgtgtacgcaatttcccaatgtacgataaacgaagtttaatatactatatagtgtacgtacacgaagacatctagtaatacttaaagtggtaattcattcaaaagccagaaacataaaaaaggacaggcagtttatataaagggaaattcataagataagataaataaactggaacgattcaaaaaacataattttttaaacgaagcatacacatgtgttgcaaaatatgtgtgcatccgtcataccaaaagactcgagagtagagccgagtagagtagcttagaccttagcagctttgcttctagtagaatatagccatctccctggcgtaccagtagaaaaaacaaaccctgcagccatgatggaacgtgcattaccctacgcaactcggcgggtggacaaacctccaagacctttcccagcggcgagcattttcagtgtgaagctggctgatgtgatacatcaccatctctgcaaccgcgtggccagcttcaagggtgggtgtcatttcccagaacttttcgctgatagggaggaaatccaagtgattaccggtacacgtaaggaacaaaccggttgcgaagcaagcaaagggatcagataaagcgacggttgtcatgactgaaaggccctgagctactgtcacgaagttgctcgcttggcgaacaccttcaaggtaaaccgccgggatggatccacgttctacaagcacctcgaagtatggccagaagggagaccccggttggagtaacgacggccacgtcatgaattcatcgattgcaagggtgttgatgtcagaactgtagactaagttccttagcgtctctatgggagtctggctgacggggaggtagttgacaagtatgcttatggggtttggcaattggattcctaggtacaactcggttggaggggttagcggaggtaggaaaggcattgtttttcaaacgaggaggtctagacggatttgtgtattcaactaagtgtgtttgtcatgagttttaaagaggagaataaccatcaatgtaagtgggtgggtgacgtggatgtatatttagtgcatcatattagtaacttttaatttaaacaaattcctgtgtacctctcataatcgcgccgtgtacgtctacaaaatactcattacgttctctgacagaaggttgagttgacgtctgtttgtatggcatataagtggaacaatatggcttcatgtatagtgctatattaggggtcaacgcatttactaacaatcttttcaaataaacattaacttacttattttatctgccaaatcaattgagtacattgctcggtaaccgtgtacgtctaatagagatattaatgagagtgtgatggaggtcacttctagagtgtatttgacagcgagtaattaatttggagcgaaatcttctatatatgggttatacgacgtcaaacaaatttccttttgtcttcatttacaaatatcgtcgctcactatgtaatctttatagttggaattaaactatgccacgtgtacattatcatatatcgtgtacgtagatattttattattttattattttaatgatgtctcatattagagtactttgagctagtatgtttcaatcaattacaaagtggtaatttaattaaaaacaaacttccattactgataatcaacgtaaaaagctcacatattgttgcatggaaaacttataaatatgacaggatcgtggcgaatgtgagctctacaggatctggctaatccgtttagcacacaaccagatgtaacattggttgcaaagattattgaagagtctcgatctaatgtaacacacctccgcgcattcaggagtgcttacgtcaacacattccgggaacgaaaaactgttagcgtatgtgtattttaaagtattaccatatttctttatatcttttagcacctcctcacaaatgtcacgtgcgtcctccgattccaaagcataatggttgcttccgaagagccgaaggtagacaccacccatgtgagcattaccagcacatatgtaaagaattgcgcatgcaagggttgcagcggcgttccttggggcgattcgctctaagtgtaggatagctccctggatgtcagactcatgtgtaacgagacgcagtccttcatggtaaatagccgtggggttaccagcttgtaagcaccgttgaaagaaggatctatagcgatcttcggagttgatgtcatttggatcgtggcctgccgcgtagaagtcatcgggatcgtcgcacatgctgaaaatgtttgcatttttgaggacatccggacggtagacaatgtctcttccacgaggaccggattttaacataggtccgaggtaccaccaacatttgtcagccattttcttggctatcttcgcaagcaaatcgtcaggaatatttgggtttgtcatatttaggagtaaggtgtttcgagaaaatgaaatttgaacacttaaataagcatcattgaagatatggttgggtaagttatggttgtatttattgcaaaggtattaagtgattatgtgtattcatattgtcaaatcaaagtaatagtattccatatataatttgttatcgttgttatgagcaacctctttttaataacagcttaaaactagacgtgtacgttttactgacggtcttagtgtacgtccacatttacatttctacatttactcaacaaacagtgtacgttgtagtgtatgttttagtgaacgtccacatttacatttctacatttgcccaacaaacagtgtacgttgtagtgtacgtttaagtgtacgtccacatttacatttctacatttgcccaacaaacagtgtacgttgtagtgtacgttttagtgtacgtccatatttacatttctacatttactcaacagacagtgtacgctgtagtgtactattagtgtacgtccattcataaatatcaccatttatgagacaaaccaaagacctcatatgtttgcatgtgttattttttagtgtacgttagagttgatatctcatgctagtgaacgtccatatctagttttccgagacaaagaaaaaacctctaagtattatttggtagatgcacgtgtacggagttgtggacgcttagattttaatatccaaatttacatttactgcagtgtctaaatatcatatgtgaatttggcggaaaaatattcaacttgagaaacataacacaccttgcaaatttcttaagcaataatataatttcaacataaacataaacaatatagtagaaggcttatcataatttgaaacataacatagcggataacataaacaaacatataaagtagaatggaataactatagcatttgactaacacgcctggcacacgaccagaggtaacagcggttgcaaacgttttggaaagctcctgataccatgtaacaatataaggcgcaaggaggcatactaattccatggctggtaggataagagaacgtaggaccatatgtattgctgtatggagggtcaaacttctttatttcctcgatgaactcatcacccaaaactcgagtggcaaccgagtccaatggataatggttgcgggtgaagagctgtagaaacaagccgcccatataatcatacccagcacatatgaatacaatggcgcatgcaagtgttgcatttgctcgtactggagcatgacgctgtaagagcctgatggctccattgatgtttcgttcatgcgttagaacacgaataccttcgtaatacacggccgtgggattattagctgcaaaacaccttaagaaaaatgttcgatgtcggccttcatcagcggatccggagggattgtggcccctccagcttggaagtcatcgggcgagtcacacaaggtgagaatattggcatctttgaggacatccggacggtagacaatgtctcttccacgagtgccggctcgtaccaatggaccaaggtcggaccagcattggtcagccaggttcttagctatcttcgagagtaaatcgtcgggaagagtcgggttaaccatgtttgaaatgagatagtcagaggtcgtcagtaatttcttcactttaataaccactactgtgaatttggttggggcaattacggttgaaatgaatgtcatacttgtgttggtaagcgttggctgtcaaatcaatttatcacttgagttatgttataaagttaatatgcagtgtacgagacatatagatatgtacgtacacatttacattatgtaattataagtgaacgatgtgtacgacaatatgatagaggacaataaattaacttctgacaatctccaaattaaatgtagttgcaatcatatatataagtcaggtttgttcataataattggttgcaaagaaccagaatattgttcttggacgattaaaccaagtgcacagtaccggcctcccgcttgccgtgctcgtagatgtcaacagagtatttctgacggtagaagaccatatctgcttctcgaatggttgtcagatccgaaaaagggtgtccaaacgcaagcaactccaggaacttcatcgtgtatggcccgcaatctcttgtagttgggttttgagcaacggtaggacaacgcacatactcaaaaggttcaactgagtacggagaaatcaatacatcctggcacatcgctctgactaagtacggcatcatttcgcaaataggagtcattctcgccttcacagccgactcccacgtgtgcgatattaaagcgtcatacacagttattagccgttcattaaggttgattcccaaaacaacccaatgttcactcttccagttcatcggagcatacacaacatcgacatccttcatccactttatattgggttcacggtttatgtgaactccattagcaatgtcggatagtaatttaccccatttgaacccgttcttattaatacacttcttgaagtcgcccacccgcttggtaagaagatgcgtcagcatcatgtccaatacaatacatctgtttgctatcatctgttccccgttcttgtgccacatcaacattgccatcatttctaaatgctgaaatgttcaaaaaatatatcagtgtacgtacacttccacgaaaaatttaatcatctatgtacaatttataacaaggaatgtacgtatataaatgcaacgtaccgtactttcgacccattcaccaggtgttagaaggttcgcaaaaaagtcgttgggcacttttcgaaacccgaaaagagtgtgaatcctgtaaaatccagaaaaatatttgataacaaccttaaaaatgatgattgagatattaaataatccattttactataatgtacttacacagagggtcctttaatgatattgctgaattcctctacttttgaagggtcaaccacgtccagggggttgtatgagacatttccagccctgataacaacctctatctgttgagctggtacatcgggcaccaatactgtttttgccttaaaaccatgaggatgggcatcatcctgttcatagacaagttgtttcttcactttctttggggacgattttttttcttcttttgcttcgacagcagcagcattgtctgattcatcctgttaacaaatatgattgtcaacaacagtttcatacgtttttctatgtttaaaataggtacatacattatttttaggaatatcggtcttgtctgcttcatctgaccctgggttaggatcctcagtttctacagacccctcgacaatacacggctcttccatgactacctgtgcaagttaacgtacacaagtatatataatgtacacatataatcctatgtatgctattccgaaaataagaattgaaacacacaaattgttacctccaaatcagtggctgcagctaaatcacaaacggctttggtatccaactcccatatgacctgcataatataaattttatacatttgaacagtaaatagtacattttatttgttgcaagagttagcttacattgttaactacttgaaatggtgacgggggatcactgtgggtcttctatggtatttcaacttccatcattgtcactaatttggattctcctgccgcctcaacctgtagaatttagcatcggaaagaaactttttagtaatgtatgaaacaatatatatgtacacagaacatagtgacaaaaatgtacgtacacttgtggcaggtacgtgagctgtgtctccatcagaaggctcataagtttcttctgactcgctgccattagcttcagtcgatgcactggcatctcttgcttcttcctgaaacaaaaataatttgcaaagtcttatgggcgtacacatatggaaaaagaacaagtgtctaccatctctatgtcagcatcagctatttcatcggcccctgtatgggtaccaactaagccaggttcagcaacatcctttggtccattatcctgaaacataggctaccatagagttaagcgttagataagtgaagaaattaatgtaatagttatgcatatagtcatggaaatctgtatataacggttaccagaagatctacctctgaaactcttgcagacacattagatggaacttcttccatctcggtgtcaacagtagcttcttccacaggcctctcttcctgtgttacaaagacagtcccagacattgaacctaagcagacagtcttaccaacaacaatatttcctctatcctgaaacatagtttaactaacttcttaagcgaatatgtacgtacaccatttaaacagattgttaagagtattagtagacgacataccagatgggattgtgtggatccagcataaggaactattaaaaatccagtgtctaatatctcttcttcagcggcagaagaattcaatccatcatttgcagtaaaatcactttcattattagcgccatcctgtatcgaaccagacagtacagttatacatatggattataaaatatttagcaatgtacgtacatgacttacatgttcatcaatgaatccttgcatatcttccttattggaatcaggttcctcaatagtgtcaacagcaaaagaaacatttttcttcggctttgaaggttttcttcttccgcagtggtatgtactccagggacatccaaatcttcctctttggaattacagccaacagaaagtgcaggagtgtccacatcatgagaaacacgagtcaaccttgaagactttctcgttccagtgcttccttttgtagacatccttctcgatggcttcttcgcagtaaccggtgccttatccatagaaataggtggttcccgcgaatcatgggaaggtatggtttgttctggccttttgccttttggatcaagggcagcacgtatctggtcacggacaaagtctttcatatcttcatgcattggatacatttggtcttccagctccttcaaagacctcctcatttcaacaataaacctctcaggcacaggctctccaaaacaatcctcagcagtctcttctttatcacagtccatttcagatttctgttttttcataacaggttctttagggctcgggcccggtgcatttcgtttcctcttaacatctacaatagaccagttgctgctattaattggttttcttctaggtgcaataccaaaaaaatttgtaaaaaaaacatttatacttatacttacatgtctcttctagttgtggtgggcgcgactgctttggtactctagcatagcctccaacccattcatcctcctgccactggtgcccatctatcaacagtgcctcaatgtaatcaacccttggatcatcaacttcatcatcccatgaaagagatggaggacaaacattatcacctggtttcacaatatagttgacctcaacctacacaaataagtatgttaaccatagataatgtacatttacttattatgtacgtacatacacaatttaacacacctcatcagcagcctcacactccaaaatgcgagatgtccggattgctcgcagagaggcaagacgatggatagacctctcagcaaatgtcctgttaggtacatcatcaggtcctagcttagcaattgatgggattgtttcgaaagctaaaagctggagagctaatgggaaaccatggagtgcataagaccctacaagtataccccttattagtttctgagcatcgtaaggagtttggaaatttgctatcctccccaaagtacgggtaaaggacacccttccccatgggtatttgcagaaaaactccaggttcttagtagtctcgacagtcttcgaagtaggcctgttggggttactgtgggccgctaccacaccgtcaaccaaaataataagagatagtgccagttgcttccacccctccatgctttcctcctcctgcagccagctcaccaaatcagcaattgttggaaccgtgtccccaaacctctcatgaaacagagtcttccaaacactctcacactcaggtttcacactgataacatcatcaacgtctttcttcttgggatacttcccacattccagcccagttaagatactaaattctcttaatctaaatctgattggatgaccgccgaatacaatccacatctcattcaccttctttgtcactaactgacggcatattagtccaaggacaagcttagcattgaaagatgccttgtttttgggtattttaaaaagtttcccaaatggagaatcgagaagaaactgcatctctggtttcccctttagtacttttgctatgtgagaaatgtactccggtttagagtacgcgttcatcttcgtctcggaagggtaccggtctagagcaaagagtcttggtggtaactcttcaactgtaagatcttcagtcggagcaacacttggactacctgcaattaaaagtcatatactgttgagcaaacagaagtagtgtacgtacacagacattttctagattataactcaaaatcatatttttggcggaaacagagatttccaattctacttctcaatttttcatcctaaagatatgctaaattccgacaaattctaacactttaatcaaacaagctttacaattctgatacacccaaagacaacaaacggtaaattagagattctaacccagttcctccgttgcctttgtcgcagagcgttttctccgcggggaaactcttaatacacgcttctccttccctggaccatgctgcttcctcgggacttcgtcgttaactttattcccagccatagctaaagatgctatttcgccggaggttcaaatttcgccggaaaaaacagtaactttctcttggcttcgccgggaaaaacagtaactttctcttggtttcgccgggaaaaacagtaactttctcttggcttcgccgggaaaaacagtaactttctcttggcttcgccgggaaaaacagtaagtttcacttcatcggatgtgggagttcagtggcacaattcaactacccaacaattcgaagttatgtcgaattttgaagcaatggatactgaatttcgtttcgctttctgattctctctcaatctctccgttattctctttttcgttttctcggtttcttcgtttcagttttttttttttttgtctttttcaaaactgaaccgcatcaactaaaccgaaaccaactgctgtagggtttggtttacgccgtcaacttaaccgatacgaactgctgcacggtttggtttgtgccattattaagttttcccaaggtaatttggtccgaaattgaatgaaaagacaaaacgtgaaaaccagttctcactttgacaaacggtgagagcaaatcttttttcgtccacttttgtgaaaatttctttttgtttaattgatgattgaaaagaaaacggcacaaaccaaacctcaatctttgttttttcctcaatgtgagtattattattagcatcacatcttctatggaaagataccaaagaatgaaaaaaaatatatatacaaaatcatataagtgtatcatatatagatcgtagttcaatagagttaaaacatttcctcaaatctttacagttaaaaagaagaattcaaaaaaaaaaaaacatgtaatttcttgcatctttaaccataaccttgaccgggtcttttaatattgccggtcgcctttccctgtgtggtctgaccgtcagtgctcgaccaggacgggtacaaatcatattcactaattgggttactatataaatcttgagtctcaagtcccactttcctaaacttgttcatgccttcattgtcttggctcgagtcataatctgtgcttcctccagatgagccatacacattgctgtgtcccggtgtgattccttggtttagatctgatggtgatatgtttccttctagcacacgtgcaacctgctcgttatttttacaccaatcactcattcgattagcattagtaacattttaaaatgactactatttagcaaattgaaaaaaggtgtagctaagaaaacatgtttgtcccattgctagggctgatcaaataagcaaaatataaggctaggcatacataccaaaccaactgagatcgaacacggtattaaccgaaaccaattcggagcggcaaccaaaacgacccaaatataaatcaatctttgtaagttatattgttccgaagccaaaaactaaactgaactgacaaatgaaaatatcttggttctaggtttctaactccgtatacagtttgaaaaccggaaagcgtaaatgcctttagagaaaagaaaagaatgattaatttacctgatccatgcgaggtctacgtggagctgtggaccgaacacaagctgcggcacaagcaaccatgcgagccatctcttctttgtcatactcattattcagttttttatcaaccacaacctcaaagtttcctaattcagatacttggttaagcaaaggtcgtgcctgttccaaattattcacacataggtttttcttcattcaaaaatcacatcgtaaacattatgtgaaggttagattatttgcatgtaattatattacttacccaatcaaccaagctgttatctgcatggacattgtttacatcaatagggcgacgtccagttattagctccagaagtacaacgccaaatgagaaaacgtcagacttttccgtgagttttccgcttgaagcatattccggagccaaatacctaaaatccaaaagtttcataatatgtaaagattcacataaacttttaacaaaactgcataaatgtagatatttggtagcttcaagattatctttacccaaaagttcccatcacacgtgtagatacatgagtatttgtatcagaagcaatcttggcaagaccaaaatcagcaacctaagaaatagaagagagacacatgatacaaagatacttcatgacaatatcgtaccaaataacaaataaaaggaagaataaatcatacctttgcttcaaatttgaaatctatcaatatgtttgacgccttgatatcacggtgaataattttaggattgcctgcatatatataaatgcctagcgagtaaacaaacatgacctagctatatatttatttattttgagaaacaaatatggaattataccatgacctatatttattacacatgatcttatagacttacaattttcatgaagataagacaatcctttggcagaaccaacagcaatcttcaatcttgaactccattccatcgtaggccgtccctttcctgtcacaaattcgattcccaagaatttactaattgaaaacattgaaggatgcaagaaacaataagagagtttattttttattttttctaaccatggaggtgaaactcgagagtgttgttgggaacaaactcatagacaagcaatctttgagcatcggcgatgcaataaccgacaagagcaaccaaatgtctatgatgtactcggctaatgatcccaacctctgcttgaaactctctttctccttgagaactcccttctttcaattgtttcacagcaacttctttcccattacgtaacatacctttgaacacgtatccgaaaccgccttgtcctaacaaattggcctcagagaatccattggtggctctagatagctcctcgtaattgaaagtgccttgatatatgcctaagcctaatgcaagccctggtgatggtggaggaagaactgattgatccgagtagtttgagtcgtagtctccgctgctgccgctgctcatgaaatgcggtggacgtggtggtgcagaggagggagacttcggtggtggcactgacatcaccacgtgatctgatcgtcgtgatgcattttgttgttgctgaccaccgtaagggactccatctgagaaacgcatgaacataatatctataaaaacggtctctctatggatccatatacaagtttaaaaatgtagaaaaaacatgtaatagaattatcataaaatgcatattcatgattaatttccccaaatatatgaaaatgacgtatatgtaaaagaaaatgtagataatctagttagcacaactatattacgttctaaatgattatatctagccgagtagaagggaaaatatagttcaagaaaatgtagataagtcatacttcaagattcaatttattttaaatctttgttttacctatgggagcagggggtgcttcattgtctcttcgtcgtttcttcttacaaaggaaaaatatcaaagccaatgccacaagaagcacaaatcctcctcctatggcgatcccgaccatagctccctttgataattctttggaagattgtgccggtggagaagagcctggagtggtagatggagtactaggagatctcccgccagagggactcgctggaggtggagggctcagactgcctacccgaggaggaggagtgggtgttgagggaggtggcggagatcttggcggagttgaaggagtggcgggagagggcggtggaggagaagaaggcctcaagggagaagacggtggaggggaaggcgtcaagggaggtgaaggaatgcttggagatggaggcggaggtgaaggggtggtgggagacggaggcagaggagatggtgtcaaaggaggtgaaggagtgatggcaggtggaggagaaggagtcaagggaggtgaaggagttgtgggagaaggcgtcaagggaggtgaaggagttgtgggagatggaggcagaggagaaggggtcaagggaggtgggggagaaggcgtagagggaggtataggaggaacggtggtaggtggaggtggaggaggaagggcgggtggaagggcagtaggcggaggtggagggggaagggcgggaggaagggcagtaggtggaggtggaggagtaacgggcagaggctgaggcggaggaggaattgggagaggctgtggaggaggtgacggagtaccaccgggaggcggcgccgatgacatatcttcccgctagtaaaaaaaaataagcgacgggataaagaagaaaaaagaatgttaaaagcgggaaaagaggtctgatctctcttgccaccgagactagacttgagttttgattacttcaatctttttaaaaaacctttggtacaccgatatagaggagaatatacgtaatcttatttttgtatgagttatatagtttgtgttcttcttgcatcaaaagcgaaagaaagaaatgagagcacttactacattgacatttctccgatgtttattattaactatctaacatcaagttacaccaaaataatccattcaaactgaaacgtacggacaataaataagtattttgttaatcatattcaatcaaggttcttaataacagctttaacaaaaaaaaaaaggttcgtaataacaggccaattatatcaatatggttctttcagatatccagataatgtttttgctcttcagataatatatgtttatgtaaataattattattgtatcagggtcaatgtcgtatcattctatatatcttgtttaaaacatcccacaagacgcttaatctaagtagaaataaaaacagaagtcaacgactgattgcaatgatactttgtcaagtttggtttggtcaaatattttgtttttgtcaaattgatttaataaaaataccagtgatatatggtccgaacaaccaattttacttaatgttggtaatagtattatttttatttttgttaacttaggaaatcttaggtcgaaactctgactatgtaataatatatagggcttataaaacatcggacaaacaacgaacgttcaaacgaagaacttaatacacgacgtcattcaagaatggatcaagcttttgttgattttcaagtgtgtatgtttacaagaaggtcaaaataatttggtagtggacattggacaagcatgccaaaatggccaaaacaaagtccactatcaattgtctacaacactataacctttttcattatgtatttgtctttttcttggaatgttttattcaacaacaacaaaattatcgtaagaaataaaaaatttgacagtgtgatataaagaaacttataacatattactttcaatcctatgtttaaaaaaaaaacactaaaaatgaaaatcattgtgctaagaatataataacacattcacattacacaaacatatatgaaaaattctaatagacccacttcgttatattagtaatacgatattatagacaagaagaaactcgatcaaacgacgcaaagctcttatataagccttaaaactcaaggaggcgcctcatgacgccaagcagctccaaatgtgatgtcatctgtggaaaatgcccgatcgcgtcctcgatgatctccactgtcgatttccccttaatcttctcctgcatgaagtatgcgacggagacaggcactacgacgtcgtttcctggttgtatgacatggcaaggcactgacacttgtcccaaaatctctctctcgtcgctaccaaacactatcttagctaaggcaagagctgtctcgggcttcatcttcttaagggacttctcgaacctttggaccgagagagagtctctcgagtcgaccacgaatgaggagaaatcaaccgcccaagcttcgtagttggagccaatgctggtaatgatcgtgtcaatgtcttttgactcaaaccctcctttgtaatcctcactgtttatatacctatttgcatttcaatctagtaacgttactaatttgatcatctaaatatcattttctctttgcgcaaaaaaaaaaaatcattttcttttatattataagtacgttgcattgatatatatagtcaaacattatcaattgttgcattaaattatggccgactttctcacctataactataataaatatctaaagtaatcagtcaaagtatgatacttcaaacagttaactcccattcgataatttctatatataggagaatatagtatgttagtggtccacgtgatatataaagatgggttaagttttagtacttctaactttaaaaatgacaataaatatattgattcatcatatgataataaaatcataacacattttttctaattttcgatgtatctgtatgaaatgaatgtcttattgacatattgatagcatatgtagcatgtttacatatattcatctacgtatatatgcgtagactgcagagaagtaagagaagatgtgatcatatgtcatatccatccaacaagctagaagctgatcatgtccacatgttcaacacttggcaacagttacacttcattacatatagatatcaaccacataaaaactattaaatattggctaatatagtatattattgtaaatcattatttttattttgtctttttcctcaaacacatattcacgaaggaatcttgtacgatcggtcagactgtataaccttataattttgattgcatgtgcttagatttctctttcgaacgtaaattataaattctaaatcatcggtcttcatcttatttacagatatattatcatattttataaatagattcaatatataggcctaaatgatgttatacagaacagaaatagattatacatatgctttgatttttacttccaattatgattaagggtaagtgttatactaagagtgaagcgtaccttggagaagcagcaataaggagaagatttgtaaacaagtcgggccttttaatagaagcagcacaaccgatcacccccgacatggaatggcctacgaacacgacagggccaaacttcaactcctccataagagcaatgagatcgtcagagaagacgtctaaggagttatactttgaagggtcatagagagtttgatctttaatggctccagagaaaagccagtcaaagaccaaaactttgaaggattgggacaagaccggtattattttatcccaaaccgactggtcgcctccgaaaccgtgtgccaagaccatcgatctctctcctgaaccgatgattttggcgttcatagcggatgcaaggccggatatcttctgattaaccaccatgcttaagtacaagagttttgttagatgggacaactttgataagttgtgtgcgtatatataaaggagaagagagatggattaggtttatggttctttaattgacattcaaccaccgattttattaggctcgcattttggtacatccaaacatttatggccttttcaacaatggctgtatttgtattcattgtccatctacatagcatcttagaggataaaacaatatccatcataaataagaatatactactccatgtgaaaacgaaactctactttttggtagtattaaaaaggtaataattatttggactcgatatgaagtatatatactagttttgtttgtatgtgtaaagtttagagtatcaaagtgtctttctttttttttggtgtgtatcaaagtgtctttttttctatcaaacaatgcatttgttgtaaaaacaatatttttttcttttaaagctaatttattattattttcgaaaaaataaatgcattaactaatctgacatttagacgtcaacatgatctcaaattctcaatacattaacataaccataaatcttaattaagttatagtcaatgaagaataaagcacacggcaaattctaataaattgattaacatcaatccttgctaatcataggagtcatctacgaaacgtataaaatgaccttttgtcttacaataataacaacgcaaattaatataattctattatatatacatcattttcgtttctctcccttagactttcgcatatatagacacatcggaaaaatataaaatacaagcaactaattgtaatcaagtttctttctttaataacaaatcttcagaagaatggatatgaaagaaaacgatatttattgtaattaaatgttttcaatttatatagtataataaaacacatatattagtatacaccaatgactcattagctatatttgatatttcaaaagcagtagctgtgcgtggagtcaatatctcgccaccaaccggggaaactggtatttaaaagtgtataaaaacggagataaaaaaaaaaggtgtacttctttgacttctcttgaaacaaaattttctagctagctgcaaatacttggtttataaactggaatccatgctctttaaatttctgtgatgaagaggaaaagcctaaaaacggaataaaaaaacagtacacgtgagatgacttggaaaatcttaaaagactcattgaaaggactgaattaagtgtgaaaatattgtgttttatatgtataaaccatatatatataacttatataacaaagtaatgcgtctgtgtaacttttcataagaaaagatacccaacaaataaaaacaatgaacaatcgtcgaaaacctaatctagggatagccaataaaaaagcctctaaaatatcgaaaccagaacgtgtaaggtccccaataacttcgcttaaaactcaaaagtactcaaatttatgtgaggtttaaattggttcaaaggtaaaaccagagatatctgggcgagaaaaaaaaagacaagaagcacacaaccgcccatgaggaatctgaaggaccagaccccaaaagttttgttttgtccataccttgtcattttcttcttccaaatttggaccaattattattttacattaaactcagaaaaggtttgaaattttcctttatcttcacagttaatgatttagagtaaatgtgtaaagcattcgaatataattactgttttcacaaaaagaagaagaataatattttaggtgttgtttttgcatgtatggatacaaaagctagcctctatgcctaattgtgttagtatagagtgatcgatactatagaagctgtacttttttccgtagataactaaagacgaggggcaaagcgattctccacaagaagagtaattccatcatatgtatagcataacttttttgactttttgacctaaagaaaaaaagcatacttatttgactaaataaattcggttctgtataaaggctgaaatactggagaaaaagcttagttttgtcgtgtagtgatcaatgtatattgacaaaagagaagagactggaaaaaagtctacgaccaacttttccttccctttggccttaacttcactcacacatcattttttaatattacccaaatttgtccattttcaaaccattaagacgcgatattgtgcattttgagattgttagcttgtagagaattaaacttcgaaaatcctagttacaattttatttcatacaaggtgtcacagatccacaccataccgttaccttgttgatatacaagttgaagttgtccttagatattatgagcttgtttcacaaatcataaggtctaaaaaaagattgcgatatgagtttgttacaatcattagaataaaagaaaaagagagtaaaagaaaataagataataaatgaggaaaatgagagttggagagtggagagactagagaatacaaattgccttttgcctttaaaagccggttcctactctctttctccgaaataaatgtcgttagcattattatcgatcgtatctatctatacacacatgcgtaccaccacgtgttttgtgctataatcatcctataattcttattaccaatctctagatttgttgttacttgttttataaactcttttgtttttcggattcatttggcacaacctatccaaaaaaaaggtttaaatgagtatgttttaaaaagaaactaatgttattgccaatctgagtactccgaagagatcaagctaaaattgtaaggaccaaataacaaacaatctttgcaaacctttttagctcaaaaggaaactactactaaacacattataagattgaacatggtgacataaatacaaactactaattacaactagcagtttaaatatgattcgtatatcattgatactaacaagattagaaaaaaaaaacactatatataatgattgattaacaagtttcttgtatcttagaaaaaagaggaaaaaacatattgattgtttttagttttatacaactttgtacacataattaacctgcaaagagagtatttcgactatgaaaggaatatgtaaatcatgaaaggaaactatttatgaaagatacaataatataaacgtgagttgttctcaaaaggcattagcctctcatttcccatcgtatttaaatgggagtattgcaaggaaaacatatatgttggtgtaaatgactttcttggttatgcagaagaatgaaattaaacttcgaaaatctccagacttacgggttagcttaagtgtgatggcaaatccagccaagtcgttgtgaaatctccctgaacattttataaaacaaaataactgtttatttttcacatatcttgaagcttagtttgaaatttttgcatggtattttggtttactatgcaacaataaaacgggtttaatacgtataatttggtgaaactaactgttttttccttacatggtccactctcaactgttttaaagagatgaaaagttatcatcattgattacgaaaatgatatctaacttctcttatacgccccacatccacacaaatgccttaatgaaaaggttatcgcgtttgcgattagaacaaaccaaaacgttaccttcaatcataatttgcgatcttgttttggttagctaatagagttatagatcaactacgtcctagtccaaaatctgtggttataactgtagtttataatagtaaaaataaaagcttacagtaaccctcttttcatcattgtaagattcataacaatgctcttaatatgaatatacaaatttcattttcaagagatatcgtaaagcgaaaacatattgttcaagaactagacttcgactggagacaatggaggaacatctactagcctttcaatttcttccaactggtcatatggagcaatctgcagaataaccaaacccaagactcataaaatttcacatgttttggtaatatctttgtttatctagttttgattgtgatttctaagttgtatttatttatacctggctaaacccgtcgggccaggttatcgagacagcgtagtttcccattggtcgtatgtcctcaggttcgatatcttccgctacatcgccatatagaactttttgctctccagtccattcatcctgtttaacaaagatccaaacattgacactttgaattcttgttttaagttttaaaagaagctttaagaagaaacagattctcaggttgaatatctttcataccacactttgtgcagatctatcatttcttctgacggttgcagggtgcagtaagaactcttcgtctgagtttggtaccttcactctaattgccttgagatacttgtcgtatgtcacggccgttgatactgtttttaacataaaatgcacagaaaagaaagagtcttttaacttaattcatgcattgatactaaaagagaaaggaaggttttttgacatggttggttgtttaccttgctggcgtatcttggcgcattgttgcactacacatacaccaagatcctggaacgttctggcaacgtcacttagaggatccgacactacttcaggagttccgctatcccccgaagcagataactaacaatcaaaaaggaatttaagaaaaactgtttagaacaatgagtccagtaatattactgttcaatcacctgatataacaaaagaattagatgaacattccaaggacatcaaaatagcttttcattcctaaagtcaaaagctaaatattcaactgtgttgtttcatagaactcttacatcatatataacttatatacataccgttggtctaatggggaggtcaaagaggtgaggtatgccgaattgcttgaccacctgcagccaaagatgtattgtcagaagttatctaaatgtgtttaacgtacatgtcgctgttgaaatattcattttggtttcttgaagtaaaactaagagacagtataaagtcagctacctcagaacctgaacctttcccaaaagggtaataacgtttcccatcagcgtcaaagtggcacatattctccacaacagcaacgcaaggcacctactccgtaaaaccaaacaggaaataaatctcggaagttcatatctgtttgttgcagagttgagaagaaaaacaagttcacaagctccatataaaagaaaaaggagagtttatgatcaaagtctggaacagggaaataaccttaagttttgagaacatccttacaccttttgcaacatcaataaacgccaacttttgaggggtggtgacaattaccgctgctgtcaatggcgcaacctgataccaagaacattatagtcatagcatctaacagaagaatttaataatattcatcatatttcagttgctagcaaagttattcagttgttacatgcaagggaaaaagcattggcacatatatatttgcattactttcattccctacatatgcgaacgaactaaaagtaaagtagaaatggttgtataatattcaaaaccgaacctggcataaggtcagttgtatatcaccagttccaggaggcatgtcgataacaagatagtccagctctcccctgcatcaaattcctcaaaaagatttataaatgtttctggaaaaaacatgaagtcaatgattttatggaaatgaacaaaccattcagttgttgtaaggagttggtttataacaccagacaccataggacctctcataatggcacgcccttgtcctgcaaatccaaatgagactagcttgacgcccatgtattctgttggaatgatggtcttcttctccgggttctattgaacatatacagaactcgaaaaacattagtcctttgtgtgttactgctatcacaaggtgagtcagagaatattaccatttccaatatacggctctcaggattgaccatggttggtagacttggaccatagacatcagcatcaaagataccaactctagcacccataccagctaatgtataagcaagatttacagctactgttgatttcccaacaccaccctgaaatgttcatctaacaacagcgtgtcatgaactaaaagggacgctagaaagaaagatacagacagattactttctatgtcacaaagaggaaataccagctactgttccgtttctatcttttccaaaaaaacttgaacgagacaataaacaaagaacacaggacatcttattgatatcacatcactaagactactttggcagaagagtatgtagcattcgtatttacacagcagagcacatcccatattattaacttaaatttgagaagaaacttcagttcaaaaagaaacatcaagagctgtgtaaaaagaaattcattggaagatatatgtaccttgcaactagaaacagcgatgatgttcgaaattcttgataatccaaagggaagctgccctgcaaaaatgggcttggctggttgtgctgacattgtcacatttaccttcttcacccatggaagggctgcaactacctcatttgccttgttctcaaactacaaaagagtcgtaatccatccatcaaagatacactaaaagaactgattcattgcttatataaccaatgatatagctccaaaatcctcctaatgggatgaagttctatcagtagtaatatgataaaagaatagtgaatggtaatgtcatttaccatgtctttgactggacatgcgggtgttgtcagctccaaacggaacgaaacctacaacaagagaccaaacatgtttcagcagccttgaacaaacaatctaaaacagaacaaattggtgaaggataaagtattattattacctcacccaaagcttcattaatccccaaatctttcacaaaaccacaagaaacaatatctgtcccaaaatcaggatcaataatctgagacagagccttcaacacatctttttctgatgtttgagcaacactctcaccaacactactactagctgttttccaaaatgcataagttaaaaaatatcagaacttgcttagtctccttcgcgtataacgttaacaatttttcaggcggagacaaaggaacaaactttataaccttgagctgaagcagctttagctacagagagattctgagagacccgtttgaggattcttgtcctggagattgagataatggaagcttgagaatggagaaacttgtgagaaagaagcaatcttgttgtggaattgcttcttctttgggtctgaatctcgaaagaaggatgccgcaacgactgtggatgaagaagcggcattgctacaagagagagacgaagagagtggcaaaaaaaactagcggtctgtgataatttgatcgctcaactctttaaaagataaaactttttcaaagctttttattatttttattattattattcaatatatcataaatttttacatttgtaatcagatattattgttttttttactgatgtgtgtcaaattacatctaagataaatcaaaattaagtttttactgatgtatgttgaattctacaaaagtttcttttgttaacacaaaatgttaaatgtttcgaaattaagataaatcaaaattaagtaaattaaaacttaaaaagacaggaaagcagatttgattgaagtaaattcagatgattaaaatgttagatttagagaaattttcaaaaaaatagactaatagtagatctagcaattaatcaagattgttattgcaccgttctaaaactacaaatcttaatctatagttaatcgactctcgttataattgactaactatgcctaacaatattggttttgtgtctccatctaaaattactagaattaagcaagcattagaattcagtttgataagtttacctaagcatctaaaccgggttggcagtcatctagttattaaggctctcctaacaataattccagtaataaacgcatctcggattttacatattaacttataatcaaattttagttcgttactctagaactagctttaagaataatcaaaggagaagaactctagggataaatactaacagattatcgatttacgatttcatctaaatttctaatgataaactttaaacccaagaaggagattactcagacataattaaagaaacataaaacatgtttgaataagattacataaagaaatcaaaagtagaatggagtttaaaaaatatcttttctctataggtataaggttttgaaatttctgaagtagtatccacaaacctcaaagaatttggagagcaaaaaatgaaataaagctt

SEQ ID NO: 5 - DNA, 368 bp, Arabidopsis thaliana
tgggacaccgtttacacacgaacgtttactctaatagagcatgtatgtatgattgtctaattccagtgtatctggtcatccttgttactgcgcatagccaacctagcggaaccccggattttgaacccgtcatcttatcaagactgattctgcgccgacctttgcgactccacggagcacaattctatggtgctattgcaatatatgcctgcatacacgtctgcatatgctggtctcatcgcgttttggaggtcttctcatcagatacctatccagaggctggctgcccaattatggtacacaatcctttttgaatttcaagctgcagcccgggccgtcgaccacgcgtgcccttagttgagtcgtat

SEQ ID NO: 6 - DNA, 703 bp, Hordeum vulgare
  misc_feature (2)..(2): n is a, c, g, or t
gnacggnaaagtcctttgactggtttgatggttctcctattgacgaacttttatgcaaggtaagggagatatatggcctggacgagaaaactagtttccgcaacgtcactatctcgttggaagggaggcctcaacctttatatcttggaactgctactcaaattggagtgatatcaactgaggggatccccagtttaccaaaaatgctactccctccaaattgtgccgggcttccgtcaatgtatattagagatcttcttcttaatcctccatcttttgatgttgcctctgcaattcaagaggcttgcaggcttatgtgcagcataacttgttcaattccagaatttacctgcataccatcagcgaagcttgtgaaactacttgagtcgaaagaggttaatcacatcgaattttgtagaataaaaaatgtccttgacgagattatgttgatgaatggaatcactgagctttcagctatccagaacaaattgctcgaacctgcttcggtggttactggcttgaaagttgatgctgatatactaattaaagaatgtagatttatttcgaaacgtataggtgaagtgatatctttagctggcgaaagtgaccaggcaatatcttcatcggaatatattcccaaggagttcttcaatgatatggagtcatcttggaaggggccgtgtgaaaagggtccatgctgaag

SEQ ID NO: 7 - protein, 232 aa, Hordeum vulgare
  misc_feature (2)..(2): Xaa can be any naturally occurring amino acid
TXKSFDWFDGSPIDELLCKVREIYGLDEKTSFRNVTISLEGRPQPLYLGTATQIGVISTEGIPSLPKMLLPPNCAGLPSMYIRDLLLNPPSFDVASAIQEACRLMCSITCSIPEFTCIPSAKLVKLLESKEVNHIEFCRIKNVLDEIMLMNGITELSAIQNKLLEPASVVTGLKVDADILIKECRFISKRIGEVISLAGESDQAISSSEYIPKEFFNDMESSWKGPCEKGPC

SEQ ID NO: 8 - DNA, 540 bp, Hordeum vulgare
ctagtgtaaatggcggcttggttgataggcctgatggtctgggaaatgggttggaacctccaacaggttcttttggactgctgcgaaaggatgtcgagagcattgttactgcgatatgcgaagacaagctgttggacctgtacaacaagagaagcatctcagagcagattgaggtggtctgtgtaactgtaggtgctagggagcaaccgccaccttcaaccgttggcaggtccagcatctatatcattatcagacgtgacaacaagctctatgttggacagacggatgatctcgtgggccgtcttggtgctcatagatccaaggaaggtatgcaagatgccacaatattatacatcgtggttcctggcaagagcgttgcgtgccaactggagactcttctcataaatcagctaccctcgaaaggttttaagctcaccaacaaggcagatggcaagcatcggaactttggtatgtctgtaacctctggagaagccatggccgcgcactgaactgccccactgaacatccagttttaactcg

SEQ ID NO: 9 - protein, 168 aa, Hordeum vulgare
SVNGGLVDRPDGLGNGLEPPTGSFGLLRKDVESIVTAICEDKLLDLYNKRSISEQIEVVCVTVGAREQPPPSTVGRSSIYIIIRRDNKLYVGQTDDLVGRLGAHRSKEGMQDATILYIVVPGKSVACQLETLLINQLPSKGFKLTNKADGKHRNFGMSVTSGEAMAAH

SEQ ID NO: 10 - DNA, 540 bp, Hordeum vulgare; identical to SEQ ID NO: 8

SEQ ID NO: 11 - DNA, 444 bp, Zea mays
taattacttccttagacaagggaaatattataactcccctggcccctactatgcacaaggctagcaccactatcagttcaacaaaactagggcggcatggtgtcagttagctcccgcctcctattgaatatccaatagcaaaaagaccttcagctgactagttccgtcgagtagcaactgcctcgccagagattcgagatataccgaagttcctgtgcttcccgtctgccttgttgatgagcttgaagcccctcgaagggagctggtttatgagaagggtttccagctggcaggcaacgctcttgccagggaccaagacgtataataccgtagcgtcccgcatgccttccttcgatctgtgggcgttcaagcgccccagaagatcgtccgtctgtccaacatagagcctgttgtcgcttctgataatcacgtagatgctagatc

SEQ ID NO: 12 - protein, 94 aa, Zea mays
SSIYVIIRSDNRLYVGQTDDLLGRLNAHRSKEGMRDATVLYVLVPGKSVACQLETLLINQLPSRGFKLINKADGKHRNFGISRISGEAVATRRN

SEQ ID NO: 13 - DNA, 338 bp, Medicago truncatula
caatggtaataattctaatgggacacatcattccgaaaagtttttatcaacaatttctcaggagggaatctctttagctaatccaattgaagtttcacataaggaggttgagagtgctatcactgtaatctgccaagattttatagcggaactgcgaaggaaaaagatcacatcataacttatcaagataaagtgtttcttaattggcactagggaatggccacctccgatgactatatgctcttcaagtgtctacgtgatgctcagaccagatcagaaactctacgtaggagagacggataatctcgaggatcgagttcgtgcacatcgatcgaaag

SEQ ID NO: 14 - DNA, 679 bp, Allium cepa
ggaatcttcatggaaaggccgtgtgaagaggatacatgctgaggatgtgtttgctgaagttgacaaagctgctcagtctttgtctattacagttatggaagactttgttccaatcgtttctagagtaaaagcggttatgtcttctcttggaggtccaaagggtgaagtatgttatgctagagaacatgaagctgtttggttcaaaggaaagcgttttatgccatctgtttgggctaatacacctggggaagagcagatcaagaaacttaaacctgccttggattcaaaaggaagaaaagtcggagaggaatggttcacaacgatcaatattgagaatgcattaactaggtatcatgaatctacggaaaaggcaagaattaaagttttggacttattaagagaactttctggagaaatgcaggctaaaattaacatccttgtcttctcttccatgctgcttgtcatatctaaatctctttttggccatgttagtgaaggtaggagaagaggatgggtgtttcctgacctgcacaattcccaaatcataaggataatagtttggacactggtaatgaaacacttgagctaagagatttatcacctttatggtttgatgctgtgcaaggaagtgcaatggaaaatactgtcagaatgcattctatgtttcttttactgggcc

SEQ ID NO: 15 - protein, 179 aa, Allium cepa
ESSWKGRVKRIHAEDVFAEVDKAAQSLSITVMEDFVPIVSRVKAVMSSLGGPKGEVCYAREHEAVWFKGKRFMPSVWANTPGEEQIKKLKPALDSKGRKVGEEWFTTINIENALTRYHESTEKARIKVLDLLRELSGEMQAKINILVFSSMLLVISKSLFGHVSEGRRRGWVFPDLHNS

SEQ ID NO: 16 - DNA, 662 bp, Citrus sinensis
attggtttgatgcagcagaaggcagtgctgtacataatacagttgatatgcagtcattatttctcctgactggtccaaatgggggtggtaaatctagtttacttagatcaatttgtgctgcttcgttacttggcatatgtggtcttatggtgcccgcagagtcagcctcaattccttactttgatgctatcatgcttcacatgaaatcctatgatagccctgctgacgggaaaagctcatttcaggtattctggttccttgtactgaggttgtaagtttgctcatgccatgatagatcgagcttagccatgatcttgtgaggcatggtagtagtaactggtgcaggtgagaaatgttgagtactacaatttacacattgcacttcacctctcatctcaaatctggtggaaaagcgtaatgtattaattttctgtggatattatatgtctgcattctcttaatttcagtatttgctgcaaaaggttatctccattaagttgcacatgttgctcagtaccttaagtttttactttgaacaagcaattttttgtatgttggaattatcttcgataggagtggtatcaagtaatatgcaaataattccgttttaatggttcaggtagaaatgtcagaaatacggtcaattgtcactgcaaccactt

SEQ ID NO: 17 - protein, 81 aa, Citrus sinensis
WFDAAEGSAVHNTVDMQSLFLLTGPNGGGKSSLLRSICAASLLGICGLMVPAESASIPYFDAIMLHMKSYDSPADGKSSFQ

SEQ ID NO: 18 - DNA, 600 bp, Solanum tuberosum
gcacacagacactgtgtattgtgcactgatatcgagcaatgtattgggttacggcaaaaaacgtcgccgtttcagttccccgttggcgttcactgtcccttttcctccgtccaccacttcgccggcgtttcttctctttctctccacatactctgtgccgagagcagatacgttgcttgaaggagcggaagttttttgccacaacggcaaaaaaaaactcaaacaaccaaaaagtgttccagaggaaaaagactatgttaatattatgtggtggaaagagagaatggaattcttgagaaagccttcttctgttctactggctaagaggcttacatattgtaacttgctgggtgtggatccgagtttgagaaatggaagtcttaaagagggaacacttaactcggagatgttgctgttcaagtcaaaatttcctcgtgaagttttgttctgtagagtaggtgatttttatgaagcaattggattcgatgcttgtattcttgtggaatatgctggtttaaatccatttggtggcctgcgctcagatagtataccaaaagctggttgtccagttgtgaatctaagacagacgttggatgatct

SEQ ID NO: 19 - protein, 187 aa, Solanum tuberosum
MYWVTAKNVAVSVPRWRSLSLFLRPPLRRRFFSFSPHTLCREQIRCLKERKFFATTAKKKLKQPKSVPEEKDYVNIMWWKERMEFLRKPSSVLLAKRLTYCNLLGVDPSLRNGSLKEGTLNSEMLLFKSKFPREVLFCRVGDFYEAIGFDACILVEYAGLNPFGGLRSDSIPKAGCPVVNLRQTLDD

SEQ ID NO: 20 - DNA, 3396 bp, Oryza sativa
atggccattcagcggctgctcgcgagctcgctcgtggccgccacgccgcggtggcttcccgtcgccgccgactcgtttctccggcgccgccaccgccctcgctgctccccgctccccgcgctgctatttaacaggaggtcctggtctaaaccaaggaaagtctcacgaagcatttccattgtgtctaggaagatgaacaaacaaggagatctctgtaatgaaggcatgctgccacatattctgtggtggaaagagaaaatggagaggtgcaggaaaccatcatcaatgcaattgactcagagacttgtgtattcaaatattttaggattggatccaactttaagaaatggaagcttgaaggatggaagcctgaacacggaaatgttgcaattcaaatcgaagtttcctcgtgaagttctactttgcagagtgggagatttctacgaggctgttgggtttgatgcatgtatccttgtggagcatgcaggcttaaatccttttggaggcttgcgttctgatagtattccaaaagctggatgtccagtcatgaatttgcggcagacattggatgatttgactcgatgtggttactctgtgtgcatagttgaagaaattcaaggcccaacccaagctcgtgctaggaaaggccgatttatttctggccatgcacatcctggtagtccttatgtatttggtcttgctgaagtagaccatgatgttgagttccctgatccaatgcctgtagttgggatttcacgatctgcaaaaggctattgcctgatttctgtgctagagacaatgaaaacatattcagctgaggagggcttaacagaggaagcagttgttactaagcttcgcatatgccgttatcatcatctataccttcatagttctttgaggaacaattcttcaggcacatcacgctggggagaatttggcgaaggtgggctattgtggggagagtgcagtggaaaatcttttgagtggtttgatggtaatcctattgaagaactgttatgcaaggtaagggaaatatatgggcttgaagagaagactgttttccgtaatgtcagtgtctcattggaagggaggcctcaacccttgtatcttggaacagctactcaaattggggtgataccaactgagggaatacccagtttgctaaaaattgttctccctccaaactttggtggccttccatcattgtatattagagatcttcttcttaaccctccatcttttgatgttgcatcatcagttcaagaggcttgcaggcttatgggtagcataacttgctcgattcctgaatttacatgcataccggcagcaaagcttgtgaaattactcgagtcaaaagaggttaatcacatcgaattttgtagaataaagaatgtcctcgatgaggtgttgttcatgggtagcaatgctgagctttctgctatcctgaataaattgcttgatcctgccgccatagttactgggttcaaagttgaagccgatatactagtgaatgaatgtagctttatttcacaacgtatagctgaagtaatctctttaggtggtgaaagtgaccaggcaataacttcatctgaatatattccgaaagagttcttcaatggtatggagtcatcttggaagggacgtgtaaaaagggtgcatgctgaagaggagttctcaaatgttgatatagctgctgaggcactgtcaacagcggtcattgaagattttctgccaattatttcaagagtaaaatctgtgatgtcctcaaatggaagttcgaagggagaaatcagttatgcaaaagagcatgaatctgtttggtttaaagggaggcgattcacaccaaatgtgtgggccaacactcctggtgaactacagataaagcaattgaagcctgcaattgactcaaaaggtagaaaggtcggagaagaatggttcaccactatcaaagttgagaatgctttaaccaggtaccatgaagcttgtgataatgcaaaacgtaaagttcttgagttgttgagaggactttcaagtgaattgcaggacaagattaatgtccttgtcttttgctcaacgatgctcatcataacaaaagcactttttggtcatgttagtgaaggacgaagaaggggttgggtgcttcctactatatctcccttgtgtaaggataatgttacagaggaaatctcaagtgaaatggaattgtcaggaacttttccttactggcttgatactaaccaagggaatgcaatactgaatgatgtccatatgcactctttgtttattcttactggtccaaacggtggtggtaaatccagtatgctgagatcagtctgtgctgctgcattacttggaatatgtggcctgatggtgccagctgcttcagctgtcatcccacatttcgattccatcatgctgcatatgaaagcatatgatagcccagctgatggtaaaagttcgtttcagattgaaatgtcagagatacgatctttagtctgccgagctacagctaggagtcttgttctaattgatgaaatatgtaggggcacagaaacagcaaaaggaacatgtatagctggtagcatcattgaaagactcgataatgttggctgcataggcatcatatcaactcatttgcatggcatttttgaccttccactgtcactccacaatactgatttcaaagctatgggaaccgaaatcatcgataggtgcattcagccaacatggaaattaatggatggcatctgtagagagagtcttgcttttcaaacagccaggaaagaaggtatgcctgacttgataattagaagagctgaggaactatatttggctatgagcacaaacagcaagcatacatcatcagctgtccaccatgaaatatccatagccaactctactgtaaatagcttggttgagaagcctaattacctgagaaatggactagagcttcaatctggttccttcggattactaagaaaagaaattgagagtgttgttaccacaatatgcaagaagaaactgttggatctctacaacaaaaggagcatctcagaactgattgaggtggtctgtgttgctgtgggtgctagggagcaacccccaccttcaactgttggcaggtccagcatttatgtaattatcagacgtgacagcaagctctatattggacagacggatgatcttgtgggtcgacttagtgctcacagatcgaaggaaggtatgcaggatgccacgatattatatattttggtacctgggaagagcattgcatgccaactggaaactcttctcataaatcagctacctttgaaaggtttcaagctcatcaacaaggcagatggcaagcatcgaaatttcggtatatctcttgtcccaggagaggcaattgccgca

SEQ ID NO: 21 - DNA, 3396 bp, Oryza sativa; identical to SEQ ID NO: 20
  CDS (1)..(3396)

SEQ ID NO: 22 - protein, 1132 aa, Oryza sativa
MAIQRLLASSLVAATPRWLPVAADSFLRRRHRPRCSPLPALLFNRRSWSKPRKVSRSISIVSRKMNKQGDLCNEGMLPHILWWKEKMERCRKPSSMQLTQRLVYSNILGLDPTLRNGSLKDGSLNTEMLQFKSKFPREVLLCRVGDFYEAVGFDACILVEHAGLNPFGGLRSDSIPKAGCPVMNLRQTLDDLTRCGYSVCIVEEIQGPTQARARKGRFISGHAHPGSPYVFGLAEVDHDVEFPDPMPVVGISRSAKGYCLISVLETMKTYSAEEGLTEEAVVTKLRICRYHHLYLHSSLRNNSSGTSRWGEFGEGGLLWGECSGKSFEWFDGNPIEELLCKVREIYGLEEKTVFRNVSVSLEGRPQPLYLGTATQIGVIPTEGIPSLLKIVLPPNFGGLPSLYIRDLLLNPPSFDVASSVQEACRLMGSITCSIPEFTCIPAAKLVKLLESKEVNHIEFCRIKNVLDEVLFMGSNAELSAILNKLLDPAAIVTGFKVEADILVNECSFISQRIAEVISLGGESDQAITSSEYIPKEFFNGMESSWKGRVKRVHAEEEFSNVDIAAEALSTAVIEDFLPIISRVKSVMSSNGSSKGEISYAKEHESVWFKGRRFTPNVWANTPGELQIKQLKPAIDSKGRKVGEEWFTTIKVENALTRYHEACDNAKRKVLELLRGLSSELQDKINVLVFCSTMLIITKALFGHVSEGRRRGWVLPTISPLCKDNVTEEISSEMELSGTFPYWLDTNQGNAILNDVHMHSLFILTGPNGGGKSSMLRSVCAAALLGICGLMVPAASAVIPHFDSIMLHMKAYDSPADGKSSFQIEMSEIRSLVCRATARSLVLIDEICRGTETAKGTCIAGSIIERLDNVGCIGIISTHLHGIFDLPLSLHNTDFKAMGTEIIDRCIQPTWKLMDGICRESLAFQTARKEGMPDLIIRRAEELYLAMSTNSKHTSSAVHHEISIANSTVNSLVEKPNYLRNGLELQSGSFGLLRKEIESVVTTICKKKLLDLYNKRSISELIEVVCVAVGAREQPPPSTVGRSSIYVIIRRDSKLYIGQTDDLVGRLSAHRSKEGMQDATILYILVPGKSIACQLETLLINQLPLKGFKLINKADGKHRNFGISLVPGEAIAA

SEQ ID NO: 23 - DNA, 433 bp, Sorghum bicolor
aaggaaggcatgcaggatgctacgatattatacatcttggttcctggcaagagcgttgcctgccagctggaaacccttctcataaatcagcttccttcgaggggcttcaagctcatcaacaaggcagacggaaagcataggaacttcggtatatctcgaatctctggagaggcaatcgccacccagctaaactaatcagctaaagatctaatttagttagtcttgacgctagtgagtctcattttgcatccttcatctcttttgcttttggctactcaataggaggcaggaactaactgacaccatatgccgccccaattttgtgagatgaattatcagtggtgctacccttgtgcatagtaggggcctagggggcgatcttcccttgtctaagcatgtagtacggtgcaaatgattagcaatgcaatgacac

SEQ ID NO: 24 - protein, 64 aa, Sorghum bicolor
KEGMQDATILYILVPGKSVACQLETLLINQLPSRGFKLINKADGKHRNFGISRISGEAIATQLN

SEQ ID NO: 25 - DNA, 667 bp, Sorghum bicolor
tggtaaatctactatgttgcgatcagtctgtgcagcttcgctgcttggaatatgtggcctgatggtaccttcaacttcagctgtaatcccgcattttgattccattatgctgcatatgaaagcctacgatagcccagccgatgggaaaagttcatttcagattgaaatgtcggagatacgtgctttagtcagccgagctactgctaggagtcttgtcctgattggtgaaatatgtaggggcacagaaactgcaaaaggaacctgtattgctggtagcatcatcgaaaggctggataatgttggctgcctaggcatcatatcaactcacctgcatgggatttttgacttgcctctctcactcagcactactgatttcaaagctatgggaactgaagtggtcgacgggtgcattcatccaacatggaaactgatggatggcatctgtagagaaagccttgcttttcaaacagccaggagggaaggcatgcctgagttcataatcagaagggctgaggagctatatttgactatgagtacaaataacaagcagaccgcatcaatggtccacaatgagcctcgtaatgacagccccagtgtaaatggcttggttgagaagcctgaatatctgaaatacaggctagaaattctgcctggtacctttgagccg

SEQ ID NO: 26 - protein, 222 aa, Sorghum bicolor
GKSTMLRSVCAASLLGICGLMVPSTSAVIPHFDSIMLHMKAYDSPADGKSSFQIEMSEIRALVSRATARSLVLIGEICRGTETAKGTCIAGSIIERLDNVGCLGIISTHLHGIFDLPLSLSTTDFKAMGTEVVDGCIHPTWKLMDGICRESLAFQTARREGMPEFIIRRAEELYLTMSTNNKQTASMVHNEPRNDSPSVNGLVEKPEYLKYRLEILPGTFEP

SEQ ID NO: 27 - DNA, 351 bp, Glycine max
  misc_feature (89)..(91): n is a, c, g, or t
ggaaatattttgttacaatcttgttacagcaaggaacacaaaaatttaatagtgtgatctttgacatgtcttccatataaagtcagtcnnncttttgcaccaagttaggcccaaattttttcatcaaagaaatagaaaagaatgagaaagtacaaaccacaagaattccgcctcaaggatgtatgcaaaaataagtaatgatattggcaagtacgaagcttcgtaacaactgcttcttctgtcaagcaatcttcagaagaatatgtcttcatggtctctagtaccatattaatgcaataacccctcgcagaatgagatattcctactacaggcattggttctgcctcgtgc

SEQ ID NO: 28 - DNA, 406 bp, Glycine max
ggaattcggcacgaggctgagctcaatgaaatattgaaacatttaatcgagcccacatgggtggcaactgggttagaaattgactttgaaaccttggttgcaggatgtgagatcgcatctagtaagattggtgaaatagtatctctggatgatgagaatgatcagaaaatcaactcgttctcttttattcctcacgaattttttgaggatatggagtctaaatggaaaggtcgaataaaaagaatccacatagatgatgtattcactgcagtggaaaaagcagctgaggccttacatatagcagtcactgaagattttgttcctgtagttgctagaataaaggctattgtagcccctctcggaggtcctaacggagaaatatcttatgctcgggagcaagaagcag

SEQ ID NO: 29 - DNA, 3393 bp, Glycine max
atgtacagggtagccacaagaaacgtcgccgttttcttccctcgttgctgttccctcgcgcactacactccttctctatttcccattttcacttcattcgctccctctcgtttccttagaataaatggatgtgtaaagaatgtgtcgagttatacggataagaaggtttcaagggggagtagtagggccaccaagaagcccaaaataccaaataacgttttagatgataaagaccttcctcacatactgtggtggaaggagaggttgcaaatgtgcagaaagttttcaactgtccagttaattgaaagacttgaattttctaatttgcttggcctgaattccaacttgaaaaatggaagtctgaaggaaggaacactcaactgggaaatgttgcaattcaagtcaaaatttccacgtcaagtattgctttgcagagttggggaattctatgaagcttggggaatagatgcttgtattcttgttgaatatgtgggtttaaatcccattggtggtctgcgatcagatagtatcccaagagctagttgtcctgtcgtgaatcttcggcagactttagatgatctgacaacaaatggttattcagtgtgcattgtggaggaggctcagggcccaagtcaagctcgatccaggaaacgtcgctttatatctgggcatgctcatcctggaaatccctatgtatatggacttgctacagttgatcatgatcttaactttccagaaccaatgcctgtagtaggaatatctcattctgcgaggggttattgcattaatatggtactagagaccatgaagacatattcttctgaagattgcttgacagaagaagcagttgttacgaagcttcgtacttgccaatatcattacttatttttgcatacatccttgaggcggaattcttgtggaacctgcaactggggagaatttggtgagggagggctattatggggagaatgtagttctagacattttgattggtttgatggcaaccctgtctccgatcttttggccaaggtaaaggaactttatagtattgatgatgaggttacctttcggaacacaactgtgtcttcaggacatagggctcgaccattaactcttggaacatctactcaaattggtgccattccaacagaaggaataccttctttgttgaaggttttacttccatcaaattgcaatggattaccagtattgtacataagggaacttcttttgaatcctccttcatatgagattgcatccaaaattcaagcaacatgcaaacttatgagcagtgtaacgtgttcaattccagaatttacatgtgtttcgtcagcaaagcttgtaaagctacttgaatggagggaggtcaatcatatggaattttgtagaataaagaatgtactggatgaaattttgcagatgtatagtacctctgagctcaatgaaatattgaaacatttaatcgagcccacatgggtggcaactgggttagaaattgactttgaaaccttggttgcaggatgtgagatcgcatctagtaagattggtgaaatagtatctctggatgatgagaatgatcagaaaatcaactcgttctcttttattcctcacgaattttttgaggatatggagtctaaatggaaaggtcgaataaaaagaatccacatagatgatgtattcactgcagtggaaaaagcagctgaggccttacatatagcagtcactgaagattttgttcctgttgtttctagaataaaggctattgtagcccctctcggaggtcctaagggagaaatatcttatgctcgggagcaagaagcagtttggttcaaaggcaaacgctttacaccgaatttgtgggctggtagccctggagaggaacaaattaaacagcttaggcatgctttagattctaaaggtagaaaggtaggggaggaatggtttaccacaccaaaggtcgaggctgcattaacaaggtaccatgaagcaaatgccaaggcaaaagaaagagttttggaaattttaaggggactcgctgctgagttgcaatacagtataaacattcttgtcttttcttccatgttgcttgttattgccaaagctttatttgctcatgcaagtgaagggagaagaaggagatgggtctttcccacgcttgtagaatcccatgggtttgaggatgtgaagtcattggacaaaacccatgggatgaagataagtggtttattgccatattggttccacatagcagaaggtgttgtgcgtaatgatgttgatatgcaatcattatttctgttgacaggaccgaatggtggtgggaaatcaagttttcttaggtcaatttgtgctgctgcactacttgggatatgtggactcatggttcctgcagaatcagccctaattccttattttgactccatcacgcttcatatgaagtcatatgatagtccagctgataaaaagagttcctttcaggttgaaatgtcagaacttcgatccatcattggcggaacaaccaacaggagccttgtacttgttgatgaaatatgccgaggaacagaaactgcaaaagggacttgcattgctggtagcatcattgaaacccttgatggaattgggtgtctgggtattgtatccactcacttgcatggaatatttactttgcccctaaacaaaaaaaacactgtgcacaaagcaatgggcacaacatccattgatggacaaataatgcctacatggaagttgacagatggagtttgtaaagaaagtcttgcttttgaaacggctaagagggaaggaattcctgagcatattgttagaagagctgaatatctttatcagttggtttatgctaaggaaatgctttttgcagaaaatttcccaaatgaagaaaagttttctacctgcatcaatgttaataatttgaatggaacacatcttcattcaaaaaggttcctatcaggagctaatcaaatggaagttttacgcgaggaagttgagagagctgtcactgtgatttgccaggatcatataaaggacctaaaatgcaaaaagattgcattggagcttactgagataaaatgtctcataattggtacaagggagctaccacctccatcggttgtaggttcttcaagcgtctatgtgatgttcagaccagataagaaactctatgtaggagagactgatgatctcgagggacgggtccgaagacatcgattaaaggaaggaatgcatgatgcatcattcctttattttcttgtcccaggtaaaagcttggcatgccaatttgaatctctgctcatcaaccaactttctggtcaaggcttccaactgagcaatatagctgatggtaaacataggaattttggcacttccaacctgtatacataa

SEQ ID NO: 30 - DNA, 3393 bp, Glycine max; identical to SEQ ID NO: 29
  CDS (1)..(3393)

SEQ ID NO: 31 - protein, 1130 aa, Glycine max
MYRVATRNVAVFFPRCCSLAHYTPSLFPIFTSFAPSRFLRINGCVKNVSSYTDKKVSRGSSRATKKPKIPNNVLDDKDLPHILWWKERLQMCRKFSTVQLIERLEFSNLLGLNSNLKNGSLKEGTLNWEMLQFKSKFPRQVLLCRVGEFYEAWGIDACILVEYVGLNPIGGLRSDSIPRASCPVVNLRQTLDDLTTNGYSVCIVEEAQGPSQARSRKRRFISGHAHPGNPYVYGLATVDHDLNFPEPMPVVGISHSARGYCINMVLETMKTYSSEDCLTEEAVVTKLRTCQYHYLFLHTSLRRNSCGTCNWGEFGEGGLLWGECSSRHFDWFDGNPVSDLLAKVKELYSIDDEVTFRNTTVSSGHRARPLTLGTSTQIGAIPTEGIPSLLKVLLPSNCNGLPVLYIRELLLNPPSYEIASKIQATCKLMSSVTCSIPEFTCVSSAKLVKLLEWREVNHMEFCRIKNVLDEILQMYSTSELNEILKHLIEPTWVATGLEIDFETLVAGCEIASSKIGEIVSLDDENDQKINSFSFIPHEFFEDMESKWKGRIKRIHIDDVFTAVEKAAEALHIAVTEDFVPVVSRIKAIVAPLGGPKGEISYAREQEAVWFKGKRFTPNLWAGSPGEEQIKQLRHALDSKGRKVGEEWFTTPKVEAALTRYHEANAKAKERVLEILRGLAAELQYSINILVFSSMLLVIAKALFAHASEGRRRRWVFPTLVESHGFEDVKSLDKTHGMKISGLLPYWFHIAEGVVRNDVDMQSLFLLTGPNGGGKSSFLRSICAAALLGICGLMVPAESALIPYFDSITLHMKSYDSPADKKSSFQVEMSELRSIIGGTTNRSLVLVDEICRGTETAKGTCIAGSIIETLDGIGCLGIVSTHLHGIFTLPLNKKNTVHKAMGTTSIDGQIMPTWKLTDGVCKESLAFETAKREGIPEHIVRRAEYLYQLVYAKEMLFAENFPNEEKFSTCINVNNLNGTHLHSKRFLSGANQMEVLREEVERAVTVICQDHIKDLKCKKIALELTEIKCLIIGTRELPPPSVVGSSSVYVMFRPDKKLYVGETDDLEGRVRRHRLKEGMHDASFLYFLVPGKSLACQFESLLINQLSGQGFQLSNIADGKHRNFGTSNLYT

SEQ ID NO: 32 - DNA, 757 bp, Saccharum officinarum
  misc_feature (512)..(512): n is a, c, g, or t
ccgcctctctcgccccccacttcccacgccccacgccgcctcccattccagttccagcgtggacgcgacgccggcgcggagacgcggcgtctcgaagcactagccccctgttgttcttccgcgccggcgcgccggcgccatgcaccgggtgctcgtgagctcgctcgtggccgccacgccgcggtggctccccctcgccgactccatcctccggcgccgccgcccgcgctgctcccctcttcccatgctgctattcgaccggaggacttggtccaagccaaggaaggtctcacgaggcatttcagtggcatctaggaaagctaacaaacagggagaatattgtgatgaaagcatgctatctcatatcatgtggtggaaagagaaaatggagaagtgcagaaaaccatcatctgtacagttgactcagaggcttgtgtattcgaatatattagggttggatccgaatttaagaaatggaagcttgaaagatggaaccctgaacatggagattntgctatttaaatcaaaatttcctcgtgaggttctactttgcagaaacatgcaggcttaaattctctttggagggttgcgttctgacagaattcctaaagctgggtgtccagccggaatttacggagacattggatgagttgactcgatgtgggaattctgtgtgcaaagtgaagaaattacaggccgacccaagccctgccccggaaagtcgattaattctgggcatgcccatcctggagcccta

SEQ ID NO: 33 - protein, 139 aa, Saccharum officinarum
  misc_feature (125)..(125): Xaa can be any naturally occurring amino acid
MHRVLVSSLVAATPRWLPLADSILRRRRPRCSPLPMLLFDRRTWSKPRKVSRGISVASRKANKQGEYCDESMLSHIMWWKEKMEKCRKPSSVQLTQRLVYSNILGLDPNLRNGSLKDGTLNMEIXLFKSKFPREVLLCR

SEQ ID NO: 34 - DNA, 504 bp, Saccharum officinarum
cacgtacctgtcctgaattccccgaccgacccatgcgtgagaacaagctttaattaaaacatacctaagtatcttctggggtcgccttcacgcccacagaggggaggaaggcatgcaagatgctaccaccctatacatcttggttcctggcaagagcgttgcctgccagctagaaacccttctcataaatcagcttccttctgagggcttcaagctcatcaacaaggtagacggaaagcataggaacttcggtatatttcgaatctctggagaggcaattgctactcaactaaactaatcacgtgaagatctaatttagctagacgacactagtgagtctcattttggctactcaataggaggcaggagctaactgacaccatgccgccccaatattgttgaactgatagcggagctagccttgaccataatacgggcatctttttctcgtctaatgatgtagtacaatgcaaatgattagcaatgcaatgacactcgttgtgc

SEQ ID NO: 35 - protein, 72 aa, Saccharum officinarum
GRLHAHRGEEGMQDATTLYILVPGKSVACQLETLLINQLPSEGFKLINKVDGKHRNFGIFRISGEAIATQLN

SEQ ID NO: 36 - DNA, 671 bp, Nicotiana tabacum
aacaattcttagccttctatgcttcagtttgtaaatgctactgttgagattttttgttgtctatttacagctggtcaagttgcttgagttgagggaggcaaatcatgtagagttctgcaaaataaagaatgtggtcgatgaaatactgcagatgtacagaaattcagagcttcgtgctattttagagtcagctgatggatcctacttgggtggcaaccgggttaaaagtcgattttgatactctagtgaatgaatgtggggagatttctggtagaatcagtgaaataatatctgtacatggtgaaagtgatcaaaagataagtccctatcctatcatcccaaatgatttttttgaagatatggagtcgccatggaaaggtcgtgtcaagaggatccatttggaggaagcatatgcagaagtagacaaggctgcagatgctttatctttggctgtgagtctctttttatttatcttcaacaatcctaatgatttacaagttgtgcatctgtgtgcgctttaatactctttcattagctaagatatacatttgctgtaaaggcagtcagcttttcaacgtccagtaaaagctttttgataaatccagtaatattatctaggaatttactgatcgatgaacaattttggggtaatcgatagacaaataaacaag

SEQ ID NO: 37 - DNA, 488 bp, Lycopersicon esculentum
gtttggtgaaggtggacttttgtggggagaatgtaatgctagacagcaggaatggttggatggcaatcctatcgatgagcttttgttcaaggtaaaagagctttatggtctcaatgatgacattccattcagaaatgtcactgttgtttcagaaaataggccccgtcctttacaccttggaactgccacacaaattggtgctattccaaccgaagggattccatgtttgttaaaggtgttgcttcctcctcattgcagtggtctaccagtcctgtatattagggatcttcttttaaatccaccaccctatgagatttcttcagacattcaagaggcatgcagacttatgatgagtgtcacatgttcaattcctgattttacctgtatttcatctgcaaagctggtcaagctgcttgagttgagggaggcaaatcacgttgagttctgcaaaataaagagcatggtcgaagagatactgcagttgtata

SEQ ID NO: 38 - DNA, 3373 bp, Lycopersicon esculentum
  misc_feature (689)..(689): n is a, c, g, or t
atgtattgggttacggcaaaaaacgtcgtcgtttcagttccccgttggcgttcactgtcccttttcctccgtccaccacttcgccggcgtttcttatctttctctccacatactctgtgccgagagcagatacgttgcgtgaaggagcggaagttttttgccacaacggcaaaaaaactcaaacaaccaaaaagtattccagaggaaaaagactatgttaatattatgtggtggaaagagagaatggaattcttgagaaagccttcttccgctcttctggctaagaggcttacatattgtaacttgctgggtgtggatccgagtttgagaaatggaagtcttaaagagggaacacttaactcggagatgttgcagttcaagtcaaaatttccacgtgaagttttgctctgtagagtaggtgatttttatgaagctattggattcgatgcttgtattcttgtggaatatgctggtttaaatccatttggtggcctgcactcagatagtataccaaaagctggttgtccagttgtgaatctaagacagacgcttgatgatctcacacgtaatggtttctctgtgtgcgtcgtggaggaagttcagggtccaactcaagctcgtgctcgtaagagtcgatttatatcagggcatgcacatccaggcagtccctatgtttttggccttgttgnagatgatcaagatcttgattttccagaaccaatgcctgttgttggaatatcccgttcagcgaaggggtattgcattatctctgtttacgagactatgaagacttactctgtggaagatggcctaactgaagaagccgtagtcaccaaacttcgtacttgtcgatgccatcatttttttttgcataattcattgaagaacaattcctcaggaacatcgcgttggggagagtttggtgaaggtggacttttgtggggagaatgtaatgctagacagcaggaatggttggatggcaatcctatcgatgagcttttgttcaaggtaaaagagctttatggtctcaatgatgacattccattcagaaatgtcactgttgtttcagaaaataggccccgtcctttacaccttggaactgccacacaaattggtgctattccaaccgaagggattccatgtttgttaaaggtgttgcttcctcctcattgcagtggtctaccagtcctgtatattagggatcttcttttaaatccaccagcctatgagatttcttcagacatacaagaggcatgcagacttatgatgagtgtcacatgttcaattcctgattttacctgtatttcatctgcaaagctggtcaagctgcttgagttgagggaggcaaatcacgttgagttctgcaaaataaagagcatggtcgaagagatactgcagttgtatagaaattcagagcttcgtgctatwgtagagttactgatggatcctacttgggtggcaactgggttgaaagttgattttgatacactagtaaatgaatgtggaaagatttcttgtagaatcagtgaaataatatccgtacatggtgaaaatgatcaaaagattagttcctatcctatcatcccaaatgatttctttgaagatatggagttgttgtggaaaggccgtgtcaagaggatccatttggaggaagcatatgcagaagtagaaaaggctgcggatgctttatctttagccataacagaagatttcctacctattatttcaagaataagggccacgatggccccacttggaggaactaaaggggagattttgtatgcccgtgagcatggagctgtatggtttaagggaaagagatttgtaccaactgtttgggctggaaccgctggagaagaacaaattaagcaactcagacctgctctagattcaaaggggaagaaggttggagaagaatggttcactacaatgagggtggaagatgcaatagctaggtatcacgaggcaagtgctagggcaaagtcaagggtcttggaattgctaaggggactttcttctgaattactatctaagatcaatatccttatctttgcatctgtcttgaatgtgatagcaaaatcattattttctcatgtgagtgaaggaagaagaagaaattggattttcccaacaatcacacaatttaacaaatgtcaggacacagaggcacttaatggaactgatggaatgaagataattggtctatctccttattggtttgatgcagcacgagggactggtgtacaggatacagtagatatgcagtccatgtttcttttaacaggtccaaatggtgggggcaaatcaagcttgctgcgttcgttgtgtgcagctgcattgctaggaatgtgtgggttcatggttccagctgaatcagctgtcattcctcattttgactcaattatgctgcatatgaaatcatatgatagtcctgttgatggaaaaagttcatttcagattgaaatgtctgaaattcggtctctgattactggtgccacttcaagaagtcttgtacttatagatgaaatatgtcgaggaacagaaacagcaaaagggacatgtattgctggaagtgtcatagaaaccctggacgaaattggctgtttgggaattgtatcaacccacttgcatggaatatttgatttacccctgaaaatcaagaagaccgtgtataaagcaatgggagctgaatatgttgacggtcaaccaataccaacttggaaactcattgatgggatctgtaaagagagtctagcatttgaaacagctcagagagaaggaattccagaaatattaatccaaagagcagaagaattgtataattcagcttacgggaatcagataccaaggaagatagaccaaataagacctcttcgttcagatattgacctcaatagcacagataacagttctgaccaattaaatggtacaagacaaatagctttggattctagcacaaagttaatgcatcgaatgggaatttcaagcaagaaacttgaagatgctatctgtcttatctgtgagaagaagttaattgagctgtataaaatgaaaaatccgtcagaaatgccaatggtgaattgcgttcttattgctgccagggaacagccggctccatcaacaattggtgcttcaagtgtctatataatgctaagacctgacaaaaagttgtatgttggacagactgatgatcttgagggcagagtacgtgctcatcgcttgaaggagggaatggaaaacgcgtcattcctatatttcttagtctctggcaagagcatcgcctgccaattggaaactcttctaataaatcaacttcctaatcatggttttcagctaacaaacgttgctgatggtaagcatcgtaattttggca

SEQ ID NO: 39 - DNA, 3373 bp, Lycopersicon esculentum; identical to SEQ ID NO: 38
  CDS (1)..(3372)
  misc_feature (689)..(689): n is a, c, g, or t

SEQ ID NO: 40 - protein, 1124 aa, Lycopersicon esculentum
  misc_feature (230)..(230): The ′Xaa′ at location 230 stands for Glu, Gly, Ala, or Val.
MYWVTAKNVVVSVPRWRSLSLFLRPPLRRRFLSFSPHTLCREQIRCVKERKFFATTAKKLKQPKSIPEEKDYVNIMWWKERMEFLRKPSSALLAKRLTYCNLLGVDPSLRNGSLKEGTLNSEMLQFKSKFPREVLLCRVGDFYEAIGFDACILVEYAGLNPFGGLHSDSIPKAGCPVVNLRQTLDDLTRNGFSVCVVEEVQGPTQARARKSRFISGHAHPGSPYVFGLVXDDQDLDFPEPMPVVGISRSAKGYCIISVYETMKTYSVEDGLTEEAVVTKLRTCRCHHFFLHNSLKNNSSGTSRWGEFGEGGLLWGECNARQQEWLDGNPIDELLFKVKELYGLNDDIPFRNVTVVSENRPRPLHLGTATQIGAIPTEGIPCLLKVLLPPHCSGLPVLYIRDLLLNPPAYEISSDIQEACRLMMSVTCSIPDFTCISSAKLVKLLELREANHVEFCKIKSMVEEILQLYRNSELRAXVELLMDPTWVATGLKVDFDTLVNECGKISCRISEIISVHGENDQKISSYPIIPNDFFEDMELLWKGRVKRIHLEEAYAEVEKAADALSLAITEDFLPIISRIRATMAPLGGTKGEILYAREHGAVWFKGKRFVPTVWAGTAGEEQIKQLRPALDSKGKKVGEEWFTTMRVEDAIARYHEASARAKSRVLELLRGLSSELLSKINILIFASVLNVIAKSLFSHVSEGRRRNWIFPTITQFNKCQDTEALNGTDGMKIIGLSPYWFDAARGTGVQDTVDMQSMFLLTGPNGGGKSSLLRSLCAAALLGMCGFMVPAESAVIPHFDSIMLHMKSYDSPVDGKSSFQIEMSEIRSLITGATSRSLVLIDEICRGTETAKGTCIAGSVIETLDEIGCLGIVSTHLHGIFDLPLKIKKTVYKAMGAEYVDGQPIPTWKLIDGICKESLAFETAQREGIPEILIQRAEELYNSAYGNQIPRKIDQIRPLRSDIDLNSTDNSSDQLNGTRQIALDSSTKLMHRMGISSKKLEDAICLICEKKLIELYKMKNPSEMPMVNCVLIAAREQPAPSTIGASSVYIMLRPDKKLYVGQTDDLEGRVRAHRLKEGMENASFLYFLVSGKSIACQLETLLINQLPNHGFQLTNVADGKHRNFG

SEQ ID NO: 41 - DNA, 622 bp, Triticum aestivum
cctactacgaacatagctaggccatatgaccaatcagacaaaattggggtggaaaacatggtatcagttagctcctgcctcctataagccaaaaaaacagataaggaaatcaaagatgaagctccactcccctttggcctctacgagttaaaactggatgttcagtgggtcagttcagtgtgcagccatggcttctccagaggttacagacataccaaagttccgatgcttgccatctgccttgttggtgagcttaaaacctttcgtgggtagctgatttatgagaagagtctccagttggcaggcaacactcttgccaggaacaatgatgtataatattgtggcatcctgcataccttccttcgatctatgagcaccaagacggcccacaagatcatccgtctgtccaacatagagcttgttgtcacgtctgatgatgatatagatgctggacctcccaacagttgaaggtggcggttgctccctagcacctacagtaacgcagaccacctcaaccagttctgagatgcttctcttgttgtagagatccaacagtttatctttgcatattgtggtaacaatgctctcgacatcctttggcagcagtccagtagcacctgac

SEQ ID NO: 42 - protein, 148 aa, Triticum aestivum
SGATGLLPKDVESIVTTICKDKLLDLYNKRSISELVEVVCVTVGAREQPPPSTVGRSSIYIIIRRDNKLYVGQTDDLVGRLGAHRSKEGMQDATILYIIVPGKSVACQLETLLINQLPTKGFKLTNKADGKHRNFGMSVTSGEAMAAH

SEQ ID NO: 43 - DNA, 523 bp, Zinnia elegans
ggagtcttcgtggaagaatcgtgttaagaagattcatttaaaagaagcttatgaagaagtggataaggcagctgaagccttatccttagctgtaacggaggattttcttcctataatttgtagaataaaagctaccacagcaccacttggaggaccaaaaggggaaattttgtatgttcgggaacacaaagctatatggttcaagggcaaacgttttgtaccaaccataggggctaatacgcctgtagaaaagcaaattaaacaacttaagccctctgtagattcaaagggtagaaaagttggagaggaatggtttaccacaagtaaagtggaggatgcactctcaaggtaccatgaagctggtgcaaaagcgaagtccatggtgttagagttattgaggggactgtctgctgaattgcaagctgaaattaatgttctcgtgtttgcctccatgttgcttattatcgcaaaggcattgtttgctcatgtgaggtattctatatctgaattttttgaccgttgt

SEQ ID NO: 44 - protein, 174 aa, Zinnia elegans
ESSWKNRVKKIHLKEAYEEVDKAAEALSLAVTEDFLPIICRIKATTAPLGGPKGEILYVREHKAIWFKGKRFVPTIGANTPVEKQIKQLKPSVDSKGRKVGEEWFTTSKVEDALSRYHEAGAKAKSMVLELLRGLSAELQAEINVLVFASMLLIIAKALFAHVRYSISEFFDRC

SEQ ID NO: 45 - DNA, 3381 bp, Phaseolus vulgaris
atgtacagggcagttaccagaaacgtcgccgttttcctgcctcgttgccgctctctctcgcacttctctcattcgctatttcccttcttcatttcatcccttccctctcgcttccttcgaataaatggacgtgtcaagaatgtatcaacttatatggataataacagggtttcaaggggaagtagtaggaccaccaagaagccaaaagtaccaaataatgttttagatgacaaagatcttcctcacatatcgtggtggaaggagaggttgcaaatgtgcaaaaagttttcgactgtccagctaattcaaaggcttgaattttctaatttgcttggtctggattccaaattgaaaaatggaagtgtgaaggaaggaacactcaactgggaaatgttgcagttcaagtcaaaatttccacgtcaagtattactctgcagagtaggggaattctatgaagcatggggaatagatgcttgtgttctagttgaatatgctggtttaaatccctgtggtggtctccaatcagatagtgttccaagggctggttgtcctgttgtgaatcttcgacagactttagatgatctgacccaaaatggttattcagtgtgcatcattgaggaagttcagggcccaactcaagctcgatccaggaaacgccgctttatatctgggcatgctcatcctggaaatccctatgtatatggacttgctgcagttgatcatgatcttaactttcctgagccaatgcctgtaataggaatatctcattctgcgaggggctattgcattaacatggtgctagagactatgaaaacatactcttatgaagattgcttgacagaggaagcaattgtgacaaagcttcgtacttgtcaatatcatcacttattcttgcatacatctttgacgcaggattcttgtggcaccagcaaatggggagaattcggtgaggggggtctcttatggggagaatgtagttctagacattttgaatggtttgatggcagccctctctctgatctcttggtcaaggtaaaggagctttatggtcttgatgatgaggttacttttcgaaacacaaccgtatcttcgagacatagggctcgacctttaacccttggaacatctactcaaattggtgccattcatacggaaggaataccttctttgttaaaggtcttactttcaccaagttgcaatggattaccggttctgtatataaggaatcttctcttgaatcctccttcttatgagatcgcatccaaaattcaggaaacatgcaaacttatgagcagtttaacgtgctcaattccagaatttacgtgtgtttcttcagcaaagcttgtaaagctacttgagtggagggaggtcaaccatatggaattttgtagaataaagaatgtgcttgatgagattttgcatatgtacaaaacctctgagctcaatgaaatattgaaaaatttaattgatccaacatgggcgacaactgggttagacatcgactttgaaacactggtttctggatgtgaagttgcatctagtaagatcagtgaaataatctctctggatggtgggaatgatcagaaaatcaactctttatctattattccttatgaattttttgaagatacggagtctaaatggaaaggtcgaataaaaagagtccatatagatgaggtgtttacagcagtgcaaaaagcagctgaggtcttgcacatagctgtcactgaagattttgttcctgttgtttctagagtaaaggctactatagccccacttggaggtcctaggggagaaatttcttatgctcgtgagcatgaggcagtttggttcagaggcaaacgctttacgccgagtttgtggtctggtagccctggggaggaacaaattaaacagcttaggcatgctttagattctaaaggtaaaagggtaggggaggaatggtttactacaccgaaggttgaggctgcattaacaaggtaccatgaagcaaatgccaaggcaacagaacgagttttggaaattttaagggaactcgctactgaattgcattacagtataaacattcttgtcttttcatccacgttgcttgttattaccaaagctttattcgctcatgcaagtgaagggagaagaaggagatgggtttttccaacacttgcagaatcgaatgggtttgaggatgtgaaatcttcggacaaaatccatgggatgaagatagttggtttagcaccttattggttccacatagcagaaggtattgtgcgtaatgatgttgatatgcaatcattatttcttttgacaggaccaaatggtggtgggaaatcaagtttacttcgttcaatttgtgctgccgcattacttggtatatgtgggctcatggttcctgcagaatctgccgtgattccttattttgactccatcacgcttcatatgaagtcgtatgatagtccagctgataaaaagagttcctttcaggtggaaatgtcagaacttagatccatcattggcggaaccaccaaaaggagccttgtacttgttgatgaaatttgccgaggaacagaaactgcaaaagggacttgtattgctggtagtatcattgaaactctagaaagaattggttgtctgggtgttgtgtccactcacttgcatggaatatttactttgcccctcaacatcaaaagcactgtgcacaaagcaatgggcacaacgtgcattgatggacaaatacttcctacatggaagctgacagatggagtctgtaaagaaagtcttgcttttgaaactgccattagggaaggaattcctgagcctattataagaagagctgaatgtctttataagtcagtttatgcagaggaaaatttcccaaatgaagagaagttttctacttgcaacaatttgaataatttgaatacaacaagtctttattctaaagggttcttatcaggagctaatcaaatggaaggttttcgccaggaagttgaaagagctattactgtgatatgccaggattatataatggaacggaaaaacaaaaagattgcattggagcttcctgagataaaatgtctcctaatcggtaagagggagcagccacctccatctgttgtaggttcttcaagcgtctatgtgattttcacgccagataagaaactctacgtaggagagacggatgatctagagggccgggttcgaagacatagattgaaagaaggtatggatgaagcatcatttctttattttcttgttccgggaaaaagcttggcatgccaatttgaatctctgctcatcaaccagctttctagtcaaggcttccaactgagcaacatggctgatggtaaacataggaattttggcacttccaacctctatgcataa

SEQ ID NO: 46 - DNA, 3381 bp, Phaseolus vulgaris; identical to SEQ ID NO: 45
  CDS (1)..(3381)

SEQ ID NO: 47 - protein, 1126 aa, Phaseolus vulgaris
MYRAVTRNVAVFLPRCRSLSHFSHSLFPFFISSLPSRFLRINGRVKNVSTYMDNNRVSRGSSRTTKKPKVPNNVLDDKDLPHISWWKERLQMCKKFSTVQLIQRLEFSNLLGLDSKLKNGSVKEGTLNWEMLQFKSKFPRQVLLCRVGEFYEAWGIDACVLVEYAGLNPCGGLQSDSVPRAGCPVVNLRQTLDDLTQNGYSVCIIEEVQGPTQARSRKRRFISGHAHPGNPYVYGLAAVDHDLNFPEPMPVIGISHSARGYCINMVLETMKTYSYEDCLTEEAIVTKLRTCQYHHLFLHTSLTQDSCGTSKWGEFGEGGLLWGECSSRHFEWFDGSPLSDLLVKVKELYGLDDEVTFRNTTVSSRHRARPLTLGTSTQIGAIHTEGIPSLLKVLLSPSCNGLPVLYIRNLLLNPPSYEIASKIQETCKLMSSLTCSIPEFTCVSSAKLVKLLEWREVNHMEFCRIKNVLDEILHMYKTSELNEILKNLIDPTWATTGLDIDFETLVSGCEVASSKISEIISLDGGNDQKINSLSIIPYEFFEDTESKWKGRIKRVHIDEVFTAVQKAAEVLHIAVTEDFVPVVSRVKATIAPLGGPRGEISYAREHEAVWFRGKRFTPSLWSGSPGEEQIKQLRHALDSKGKRVGEEWFTTPKVEAALTRYHEANAKATERVLEILRELATELHYSINILVFSSTLLVITKALFAHASEGRRRRWVFPTLAESNGFEDVKSSDKIHGMKIVGLAPYWFHIAEGIVRNDVDMQSLFLLTGPNGGGKSSLLRSICAAALLGICGLMVPAESAVIPYFDSITLHMKSYDSPADKKSSFQVEMSELRSIIGGTTKRSLVLVDEICRGTETAKGTCIAGSIIETLERIGCLGVVSTHLHGIFTLPLNIKSTVHKAMGTTCIDGQILPTWKLTDGVCKESLAFETAIREGIPEPIIRRAECLYKSVYAEENFPNEEKFSTCNNLNNLNTTSLYSKGFLSGANQMEGFRQEVERAITVICQDYIMERKNKKIALELPEIKCLLIGKREQPPPSVVGSSSVYVIFTPDKKLYVGETDDLEGRVRRHRLKEGMDEASFLYFLVPGKSLACQFESLLINQLSSQGFQLSNMADGKHRNFGTSNLYA

SEQ ID NO: 48 - DNA, 28 bp, Artificial
  primer
ggccatggtgtgaattgcatagtcgtcg

SEQ ID NO: 49 - DNA, 28 bp, Artificial
  primer
ggccatggaaacatcacttgacgtcttc

SEQ ID NO: 50 - DNA, 15 bp, Arabadopsis thaliana
agtggttgtttgggt

SEQ ID NO: 51 - DNA, 15 bp, Arabadopsis thaliana
agtggttatttgggt

SEQ ID NO: 52 - DNA, 15 bp, Arabadopsis thaliana
gatgttgcagtttaa

SEQ ID NO: 53 - DNA, 15 bp, Arabadopsis thaliana
gatgttgtagtttaa

SEQ ID NO: 54 - DNA, 13 bp, Arabadopsis thaliana
tactcagagattg

SEQ ID NO: 55 - DNA, 13 bp, Arabadopsis thaliana
tactcaaagattg

SEQ ID NO: 56 - protein, 17 aa, Escherichia coli
LLFYRMGDFYELFYDDA

SEQ ID NO: 57 - protein, 17 aa, Saccharomyces cerevisiae
VVLTQMGSFYELYFEQA

SEQ ID NO: 58 - protein, 17 aa, Arabadopsis thaliana
VVFFKMAKFYELFEMDA

SEQ ID NO: 59 - protein, 17 aa, Arabadopsis thaliana
VLLCRVGEFYEAIGIDA

SEQ ID NO: 60 - protein, 17 aa, Artificial
  consensus
LLFYRMGDFYELFYDDA

SEQ ID NO: 61 - protein, 173 aa, Escherichia coli
DKPGIRITEGRHPVVEQVLNEPFIANPLNNSPQRRMLIITGPNMGGKSTYMRQTALIALMAYIGSYVPAQKVEIGPIDRIFTRVGAADDLASGRSTFMVEMTETANILHNATEYSLVLMDEIGRGTSTYDGLSLAWCAENLANKIKALTLFATHYFELTQLPEKMEGZVANVH

SEQ ID NO: 62 - protein, 177 aa, Saccharomyces cerevisiae
ESNKLEVVNGRHLMVEEGLSARSLETFTANNCELAKDNLWVITGPNMGGKSTFLRQNAIIVILAQIGCFVPCSKARVGIVDKLFSRVGSADDLYNEMSTFMVZEMIETSFILQGATERSLAILDEIGRGTSGKEGISIAYATLKYLLENNQCRTLFATHFGQELKQIDNKCSKGMSE

SEQ ID NO: 63 - protein, 177 aa, Arabadopsis thaliana
GVPHLSATGLGHPVLRGDSLGRGSFVPNNVKIGGAEKASFILLTGPNMGGKSTLLRQVCLAVILAQIGADVPAETFEVSPVDKICVRMGAKDHIMAGQSTFLTELSETAVMLTSATRNSLVVLDELGRGTATSDGQAIAESVLEHFIEKVQCRGFFSTHYHRLSVDYQTNPKVSLCH

SEQ ID NO: 64 - protein, 177 aa, Arabadopsis thaliana
LDEGAKPLDGASRMKLTGLSPYWFDVSSGTAVHNTVDMQSLFLLTGPNGGGKSSLLRSICAAALLGISGLMVPAESACIPHFDSIMLHMKSYDSPVDGKSSFQVEMSEIRSIVSQATSRSLVLIDEICRGTETAKGTCIAGSVVESLDTSGCLGIVSTHLHGIFSLPLTAKNITYKA

SEQ ID NO: 65 - protein, 1558 aa, Artificial
  consensus
SYIRKRSSKKLKPVLDDKDLPHILWWKERLQCRKPSTVQLIRLYSNLLGLDPSLRNGSLKEGTLNWEMLQFKSKFPREVLLCRVGEFYEAIGIDACILVEYAGLNPFGGLRSDSIPKAGCPVVNLRQTLDDLTRNGYSVCIVEEVQGPTQARSRKRFISGHAHPGSPYVYGLAVDHDLDFPEPMPVVGISRSARGYCIISVLETMKTYSEDGLTEEAVVTKLRTCRYHHLFLHTSLRNNSSGTSRWGEFGEGGLLWGECSSRFEWFDGNPISELLKVKELYGLDDEVTFRNVTVSSRPRPLHLGTATQIGAIPTEGIPSLLKVLLPPCGLPVLYIRDLLLNPPSYEIASKIQETCKLMSSVTCSIPEFTCVSSAKLVKLLEREVNHIEFCRIKNVLDEILMYRSELEILKLIDPTWVATGXXMYRVXTRNVVVSXPRWRXXXXFXXSSFXXFXSXXPSRXLXINGXVXNXXSYIRXXKXXRXXSXXSKKLKXPXXVLDDKDLPHILWWKERLQXCRKPSTVQLIXRLXYSNLLGLDPSLRNGSLKEGTLNWEMLQFKSKFPREVLLCRVGEFYEAIGIDACILVEYAGLNPFGGLRSDSIPKAGCPVVNLRQTLDDLTRNGYSVCIVEEVQGPTQARSRKXRFISGHAHPGSPYVYGLAXVDHDLDFPEPMPVVGISRSARGYCIISVLETMKTYSXEDGLTEEAVVTKLRTCRYHHLFLHTSLRNNSSGTSRWGEFGEGGLLWGECSSRXFEWFDGNPISELLXKVKELYGLDDEVTFRNVTVSSXXRPRPLHLGTATQIGAIPTEGIPSLLKVLLPPXCXGLPVLYIRDLLLNPPSYEIASKIQETCKLMSSVTCSIPEFTCVSSAKLVKLLEXREVNHIEFCRIKNVLDEILXMYRXSELXEILKXLIDPTWVATGLKIDFDTLVNECXXASXKISEIISLDGENXDQKISSXXXIPXEFFEDMESXWKGRVKRIHIEEXFTXVEKAAEALSIAVTEDFLPIISRIKATMAPLGGPKGEISYAREHEAVWFKGKRFTPSLWAGTPGEEQIKQLRPALDSKGKKVGEEWFTTPKVEXALTRYHEAXAKAKXRVLELLRGLSSELQXKINILVFASMLLVITKALFAHASEGRRRRWVFPTLXXXXXXEDXKSLDXTXGMKISGLSPYWFDIAXGXAVXNDVDMQSLFLLTGPNGGGKSSLLRSICAAALLGICGLMVPAESAVIPHFDSIMLHMKSYDSPADGKSSFQVEMSEIRSIIXGATSRSLVLIDEICRGTETAKGTCIAGSIIETLDXIGCLGIVSTHLHGIFTLPLXIKNTVHKAMGTEXIDGQIIPTWKLTDGVCKESLAFETAKREGIPEXIIRRAEXLYXSVYAXXXXXXXXXXXXEKXSXXINIXNLXTTSLXXXXXXXXXXXXXXAXMXILRKELERAITVICXKKIIELXXKKXXXELXEIXCLLIGAREQPPPSTVGSSSVYVMXRPDKKLYVGQTDDLEGRVRAHRLKEGMXDASFLYFLVPGKSIACQLETLLINQLXXQGFQLSNIADGKHRNFGTSXL